(19) United States
(12) Patent Application Publication
(10) Pub. No.: US 2009/0274686 A1
(43) Pub. Date: Nov. 5, 2009

(54) NUCLEOSIDE PHOSPHONATE DERIVATIVES

(76) Inventors: Yat Sun Or, Watertown, MA (US); Lu Ying, Belmont, MA (US); Xiaowen Peng, Auburndale, MA (US); Ce Wang, Waltham, MA (US); Yao-Ling Qiu, Andover, MA (US)

Correspondence Address: ELMORE PATENT LAW GROUP, PC, 515 Groton Road, Unit 1R, Westford, MA, 01886, US

(21) Appl. No.: 12/434,146

(22) Filed: May 1, 2009

Related U.S. Application Data
(60) Provisional application No. 61/050,035, filed on May 2, 2008.
(60) Provisional application No. 61/073,176, filed on Jun. 17, 2008.

(51) Int. Cl. A61K 31/7064; C07H 19/20; C07H 19/10; A61K 31/7076; A61K 39/395; A61P 35/00; A61P 31/12

(52) U.S. Cl. 424/130.1; 536/26.7; 536/26.8; 514/48; 514/49; 424/94.63

(57) ABSTRACT

The present invention discloses compounds of formula (I) or (II), or pharmaceutically acceptable salts, esters, or prodrugs thereof:

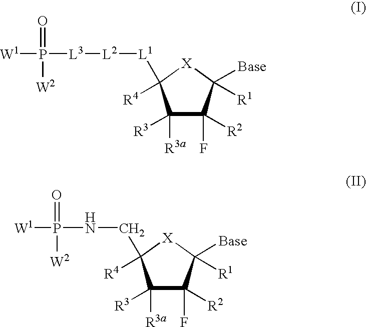

which inhibit, preventing or treating abnormal cellular proliferation and/or a viral infection, particularly by HIV, HCV or HBV. Consequently, the compounds of the present invention interfere with the replication cycle of a virus and are also useful as antiviral agents, or interfere with host cellular biochemical process and are also useful as antiproliferative agents. The present invention further relates to pharmaceutical compositions comprising the aforementioned compounds for administration to a subject suffering from viral infection and/or cell proliferation. The invention also relates to methods of treating a viral infection and/or cell proliferation in a subject by administering a pharmaceutical composition comprising the compounds of the present invention. The present invention relates to novel antiviral/anti-proliferative compounds represented herein above, pharmaceutical compositions comprising such compounds, and methods for the treatment or prophylaxis of viral infection in a subject in need of such therapy with said compounds.

### RELATED APPLICATIONS

This application claims the benefit of U.S. Provisional Application No. 61/050,035, filed on May 2, 2008 and U.S. Provisional Application No. 61/073,176, filed on Jun. 17, 2008. The entire teachings of the above applications are incorporated herein by reference.

### TECHNICAL FIELD

The present invention relates generally to compounds and pharmaceutical compositions useful as anti-infective agents. Specifically, the present invention relates to 2′-fluoronucleoside phosphonate derivatives and methods for their preparation and use.

### BACKGROUND OF THE INVENTION

Synthetic nucleosides such as 5-iodouracil and 5-fluorouracil have been used for the treatment of cancer for many years. Since the 1980's, synthetic nucleosides have also been a focus of interest for the treatment of HIV and hepatitis.

In 1981, acquired immune deficiency syndrome (AIDS) was identified as a disease that severely compromises the human immune system, and that almost without exception leads to death. In 1983, the etiological cause of AIDS was determined to be the human immunodeficiency virus (HIV). In 1985, it was reported that the synthetic nucleoside 3′-azido-3′-deoxythymidine (AZT) inhibits the replication of human immunodeficiency virus. Since then, a number of other synthetic nucleosides, including 2′,3′-dideoxyinosine (DDI), 2′,3′-dideoxycytidine (DDC), and 2′,3′-dideoxy-2′,3′-didehydrothymidine (D4T), have been proven to be effective against HIV. After cellular phosphorylation to the 5′-triphosphate by cellular kinases, these synthetic nucleosides are incorporated into a growing strand of viral DNA, causing chain termination due to the absence of the 3′-hydroxyl group. They can also inhibit the viral enzyme reverse transcriptase.

The success of various synthetic nucleosides in inhibiting the replication of HIV in vivo or in vitro has led a number of researchers to design and test nucleosides that substitute a heteroatom for the carbon atom at the 3′-position of the nucleoside. European Patent Publication No. 0 337 713 and U.S. Pat. No. 5,041,449, assigned to BioChem Pharma, Inc., disclose 2-substituted-4-substituted-1,3-dioxolanes that exhibit antiviral activity. U.S. Pat. No. 5,047,407 and European Patent Publication No. 0 382 526, also assigned to BioChem Pharma, Inc., disclose that a number of 2-substituted-5-substituted-1,3-oxathiolane nucleosides have antiviral activity, and specifically report that 2-hydroxymethyl-5-(cytosin-1-yl)-1,3-oxathiolane (referred to below as BCH-189) has approximately the same activity against HIV as AZT, with little toxicity.

It has also been disclosed that cis-2-hydroxymethyl-5-(5-fluorocytosin-1-yl)-1,3-oxathiolane (“FTC”) has potent HIV activity. Schinazi, et al., “Selective Inhibition of Human Immunodeficiency viruses by Racemates and Enantiomers of cis-5-Fluoro-1-[2-(Hydroxymethyl)-1,3-Oxathiolane-5-yl]-Cytosine” Antimicrobial Agents and Chemotherapy, November 1992, 2423-2431. See also U.S. Pat. Nos. 5,210,085; 5,814,639; and 5,914,331.

Another virus that causes a serious human health problem is the hepatitis B virus (referred to below as “HBV”). HBV is second only to tobacco as a cause of human cancer. The mechanism by which HBV induces cancer is unknown. It is postulated that it may directly trigger tumor development, or indirectly trigger tumor development through chronic inflammation, cirrhosis, and cell regeneration associated with the infection.

After a two to six month incubation period in which the host is unaware of the infection, HBV infection can lead to acute hepatitis and liver damage, that causes abdominal pain, jaundice, and elevated blood levels of certain enzymes. HBV can cause fulminant hepatitis, a rapidly progressive, often fatal form of the disease in which massive sections of the liver are destroyed.

Patients typically recover from acute hepatitis. In some patients, however, high levels of viral antigen persist in the blood for an extended, or indefinite, period, causing a chronic infection. Chronic infections can lead to chronic persistent hepatitis. Patients infected with chronic persistent HBV are most common in developing countries. By mid-1991, there were approximately 225 million chronic carriers of HBV in Asia alone, and worldwide, almost 300 million carriers. Chronic persistent hepatitis can cause fatigue, cirrhosis of the liver, and hepatocellular carcinoma, a primary liver cancer.

In western industrialized countries, high risk groups for HBV infection include those in contact with HBV carriers or their blood samples. The epidemiology of HBV is very similar to that of acquired immune deficiency syndrome, which accounts for why HBV infection is common among patients with AIDS or AIDS related complex. However, HBV is more contagious than HIV.

Both FTC and 3TC exhibit activity against HBV. Furman, et al., “The Anti-Hepatitis B Virus Activities, Cytotoxicities, and Anabolic Profiles of the (−) and (+) Enantiomers of cis-5-Fluoro-1-[2-(Hydroxymethyl)-1,3-oxathiolane-5-yl]-Cytosine” Antimicrobial Agents and Chemotherapy, December 1992, pp. 2686-2692; and Cheng, et al., Journal of Biological Chemistry, Volume 267(20), pp. 13938-13942 (1992). Other compounds that exhibit activity against HBV in humans include Clevudine or CLV (L-FMAU) (Pharmasset, Inc. under license from The University of Georgia Research Foundation and Yale University), and L-dT and L-dC (Idenix Pharmaceuticals, Inc.).

HCV is the major causative agent for post-transfusion and for sporadic non A, non B hepatitis (Alter, H. J. (1990) J. Gastro. Hepatol. 1:78-94; Dienstag, J. L. (1983) Gastro 85:439-462). Despite improved screening, HCV still accounts for at least 25% of the acute viral hepatitis in many countries (Alter, H. J. (1990) supra; Dienstag, J. L. (1983) supra; Alter M. J. et al. (1990a) J.A.M.A. 264:2231-2235; Alter M. J. et al (1992) N. Engl. J. Med. 327:1899-1905; Alter, M. J. et al. (1990b) N. Engl. J. Med. 321:1494-1500). Infection by HCV is insidious in a high proportion of chronically infected (and infectious) carriers who may not experience clinical symptoms for many years. The high rate of progression of acute infection to chronic infection (70-100%) and liver disease (>50%), its world-wide distribution and lack of a vaccine make HCV a significant cause of morbidity and mortality. Currently, there are three types of interferon and a combination of interferon and ribavirin used to treat hepatitis C. Selection of patients for treatment may be determined by biochemical, virologic, and when necessary, liver biopsy findings, rather than presence or absence of symptoms.

Interferon is given by injection, and may have a number of side effects including flu-like symptoms including headaches, fever, fatigue, loss of appetite, nausea, vomiting, depression and thinning of hair. It may also interfere with the production of white blood cells and platelets by depressing the bone marrow. Periodic blood tests are required to monitor blood cells and platelets. Ribavirin can cause sudden, severe anemia, and birth defects so women should avoid pregnancy while taking it and for 6 months following treatment. The severity and type of side effects differ for each individual. Treatment of children with HCV is not currently approved but is under investigation. While 50-60% of patients respond to treatment initially, lasting clearance of the virus occurs in only about 10-40% of patients. Treatment may be prolonged and given a second time to those who relapse after initial treatment. Re-treatment with bioengineered consensus interferon alone results in elimination of the virus in 58% of patients treated for one year. Side effects occur but the medication is usually well tolerated. Combined therapy (interferon and ribavirin) shows elimination of the virus in 47% after 6 months of therapy. Side effects from both drugs may be prominent.

A tumor is an unregulated, disorganized proliferation of cell growth. A tumor is malignant, or cancerous, if it has the properties of invasiveness and metastasis. Invasiveness refers to the tendency of a tumor to enter surrounding tissue, breaking through the basal laminas that define the boundaries of the tissues, thereby often entering the body's circulatory system. Metastasis refers to the tendency of a tumor to migrate to other areas of the body and establish areas of proliferation away from the site of initial appearance.

Cancer is now the second leading cause of death in the United States. Over 8,000,000 persons in the United States have been diagnosed with cancer, with 1,208,000 new diagnoses expected in 1994. Over 500,000 people die annually from the disease in this country.

Cancer is not fully understood on the molecular level. It is known that exposure of a cell to a carcinogen such as certain viruses, certain chemicals, or radiation, leads to DNA alteration that inactivates a “suppressive” gene or activates an “oncogene.” Suppressive genes are growth regulatory genes, which upon mutation, can no longer control cell growth. Oncogenes are initially normal genes (called prooncongenes) that by mutation or altered context of expression become transforming genes. The products of transforming genes cause inappropriate cell growth. More than twenty different normal cellular genes can become oncongenes by genetic alteration. Transformed cells differ from normal cells in many ways, including cell morphology, cell-to-cell interactions, membrane content, cytoskeletal structure, protein secretion, gene expression and mortality (transformed cells can grow indefinitely).

All of the various cell types of the body can be transformed into benign or malignant tumor cells. The most frequent tumor site is lung, followed by colorectal, breast, prostate, bladder, pancreas and then ovary. Other prevalent types of cancer include leukemia, central nervous system cancers, including brain cancer, melanoma, lymphoma, erythroleukemia, uterine cancer, and head and neck cancer.

Cancer is now primarily treated with one or a combination of three means of therapies: surgery, radiation and chemotherapy. Surgery involves the bulk removal of diseased tissue. While surgery is sometimes effective in removing tumors located at certain sites, for example, in the breast, colon and skin, it cannot be used in the treatment of tumors located in other areas, such as the backbone, or in the treatment of disseminated neoplastic conditions such as leukemia.

Chemotherapy involves the disruption of cell replication or cell metabolism. It is used most often in the treatment of leukemia, as well as breast, lung, and testicular cancer.

There are five major classes of chemotherapeutic agents currently in use for the treatment of cancer: natural products and their derivatives; anthacyclines; alkylating agents; antiproliferatives (also called antimetabolites); and hormonal agents. Chemotherapeutic agents are often referred to as antineoplastic agents.

The alkylating agents are believed to act by alkylating and cross-linking guanine and possibly other bases in DNA, arresting cell division. Typical alkylating agents include nitrogen mustards, ethyleneimine compounds, alkyl sulfates, cisplatin and various nitrosoureas. A disadvantage with these compounds is that they not only attack malignant cells, but also other cells which are naturally dividing, such as those of bone marrow, skin, gastrointestinal mucosa, and fetal tissue.

Antimetabolites are typically reversible or irreversible enzyme inhibitors, or compounds that otherwise interfere with the replication, translation or transcription of nucleic acids.

Several synthetic nucleosides have been identified that exhibit anticancer activity. A well known nucleoside derivative with strong anticancer activity is 5-fluorouracil. 5-Fluorouracil has been used clinically in the treatment of malignant tumors, including, for example, carcinomas, sarcomas, skin cancer, cancer of the digestive organs, and breast cancer. 5-Fluorouracil, however, causes serious adverse reactions such as nausea, alopecia, diarrhea, stomatitis, leukocytic thrombocytopenia, anorexia, pigmentation and edema. Derivatives of 5-fluorouracil with anti-cancer activity have been described in U.S. Pat. No. 4,336,381, and in Japanese patent publication Nos. 50-50383, 50-50384, 50-64281, 51-146482, and 53-84981.

U.S. Pat. No. 4,000,137 discloses that the peroxidate oxidation product of inosine, adenosine or cytidine with methanol or ethanol has activity against lymphocytic leukemia.

Cytosine arabinoside (also referred to as Cytarabin, araC, and Cytosar) is a nucleoside analog of deoxycytidine that was first synthesized in 1950 and introduced into clinical medicine in 1963. It is currently an important drug in the treatment of acute myeloid leukemia. It is also active against acute lymphocytic leukemia, and to a lesser extent, is useful in chronic myelocytic leukemia and non-Hodgkin's lymphoma. The primary action of araC is inhibition of nuclear DNA synthesis. Handschumacher, R. and Cheng, Y., “Purine and Pyrimidine Antimetabolites” Cancer Medicine, Chapter XV-I, 3rd Edition, Edited by J. Holland, et al., Lea and Febigol, publishers.

5-Azacytidine is a cytidine analog that is primarily used in the treatment of acute myelocytic leukemia and myelodysplastic syndrome.

2-Fluoroadenosine-5′-phosphate (Fludara, also referred to as FaraA) is one of the most active agents in the treatment of chronic lymphocytic leukemia. The compound acts by inhibiting DNA synthesis. Treatment of cells with F-araA is associated with the accumulation of cells at the G1/S phase boundary and in S phase; thus, it is a cell cycle S phase-specific drug. Incorporation of the active metabolite, F-araATP, retards DNA chain elongation. F-araA is also a potent inhibitor of ribonucleotide reductase, the key enzyme responsible for the formation of dATP.

2-Chlorodeoxyadenosine is useful in the treatment of low grade B-cell neoplasms such as chronic lymphocytic leukemia, non-Hodgkins' lymphoma, and hairy-cell leukemia.

In designing new nucleosides, there have been a number of attempts to incorporate a fluoro substituent into the carbohydrate ring of the nucleoside. Fluorine has been suggested as a substituent because it might serve as an isopolar and isosteric mimic of a hydroxyl group as the C—F bond length (1.35 Å) is so similar to the C—O bond length (1.43 Å) and because fluorine is a hydrogen bond acceptor. Fluorine is capable of producing significant electronic changes in a molecule with minimal steric perturbation. The substitution of fluorine for another group in a molecule can cause changes in substrate metabolism because of the high strength of the C—F bond (116 kcal/mol vs. C—H=100 kcal/mol).

A number of references have reported the synthesis and use of 2′-arabinofluoro-nucleosides (i.e., nucleosides in which a 2′-fluoro group is in the “up”-configuration). There have been several reports of 2′-fluoro-β-D-arabinofuranosyl nucleosides that exhibit activity against hepatitis B and herpes. See, for example, U.S. Pat. No. 4,666,892 to Fox, et al.; U.S. Pat. No. 4,211,773 to Lopez, et al; Su, et al., Nucleosides. 136. Synthesis and Antiviral Effects of Several 1-(2-Deoxy-2-fluoro-β-D-arabinofuranosyl)-5-alkyluracils. Some Structure-Activity Relationships, J. Med. Chem., 1986, 29, 151-154; Borthwick, et al., Synthesis and Enzymatic Resolution of Carbocyclic 2′-Ara-fluoro-Guanosine: A Potent New Anti-Herpetic Agent, J. Chem. Soc., Chem. Commun, 1988, 656-658; Wantanabe, et al., Synthesis and Anti-HIV Activity of 2′-“Up”-Fluoro Analogues of Active Anti-AIDS Nucleosides 3′-Azido-3′-deoxythymidine (AZT) and 2′,3′-deoxythymidine (DDT) and 2′,3′-dideoxycytidine (DDC), J. Med. Chem. 1990, 33, 2145-2150; Martin, et al., Synthesis and Antiviral Activity of Monofluoro and Difluoro Analogues of Pyrimidine Deoxyribonucleosides against Human Immunodeficiency Virus (HIV-1), J. Med. Chem. 1990, 33, 2137-2145; Sterzycki, et al., Synthesis and Anti-HIV Activity of Several 2′-Fluoro-Containing Pyrimidine Nucleosides, J. Med. Chem. 1990, 33, 2150-2157 as well as EPA 0 316 017. Sterzycki, et al.; and Montgomery, et al., 9-(2-Deoxy-2-fluoro-β-D-arabinofuranosyl)-guanine: A Metabolically Stable Cytotoxic Analogue of 2′-Deoxyguanosine. U.S. Pat. No. 5,246,924 discloses a method for treating a hepatitis infection that includes the administration of 1-(2′-deoxy-2′-fluoro-β-D-arabinofuranosyl)-3-ethyluracil), also referred to as “FEAU.” U.S. Pat. No. 5,034,518 discloses 2-fluoro-9-(2-deoxy-2-fluoro-β-D-arabino-furanosyl)adenine nucleosides which exhibit anticancer activity by altering the metabolism of adenine nucleosides by reducing the ability of the compound to serve as a substrate for adenosine. EPA 0 292 023 discloses that certain 1-D-2′-fluoroarabinonucleosides are active against viral infections.

U.S. Pat. No. 5,128,458 discloses β-D-2′,3′-dideoxy-4′-thioribonucleosides as antiviral agents. U.S. Pat. No. 5,446,029 discloses that 2′,3′-dideoxy-3′-fluoro-nucleosides have anti-hepatitis activity.

European Patent Publication No. 0 409 227 A2 discloses certain 3′-substituted β-D-pyrimidine and purine nucleosides for the treatment of hepatitis B.

It has also been disclosed that L-FMAU (2′-fluoro-5-methyl-β-L-arabinofuranosyl-uracil) a potent anti-HBV and anti-EBV agent. See Chu, et al., “Use of 2′-Fluoro-5-methyl-β-L-arabinofuranosyluracil as a Novel Antiviral Agent for Hepatitis B Virus and Epstein-Barr Virus”, Antimicrobial Agents and Chemotherapy, 1995, 39, 979-981; Balakrishna, et al., “Inhibition of Hepatitis B Virus by a Novel L-Nucleoside, 2′-Fluoro-5-Methyl-β-L-arabinofuranosyl Uracil”, Antimicrobial Agents and Chemotherapy, 1996, 40, 380-356; U.S. Pat. Nos. 5,587,362; 5,567,688; and 5,565,438.

U.S. Pat. Nos. 5,426,183 and 5,424,416 disclose processes for preparing 2′-deoxy-2′,2′-difluoronucleosides and 2′-deoxy-2′-fluoro nucleosides. See also “Kinetic Studies of 2′,2′-difluorodeoxycytidine (Gemcitabine) with Purified Human Deoxycytidine Kinase and Cytidine Deaminase”, Biochemical Pharmacology, 1993, 45, 4857-4861.

U.S. Pat. No. 5,446,029 to Eriksson, et al., discloses that certain 2′,3′-dideoxy-3′-fluoronucleosides have hepatitis B activity. U.S. Pat. No. 5,128,458 discloses certain 2′,3′-dideoxy-4′-thioribonucleosides wherein the 3′-substituent is H, azide or fluoro. WO 94/14831 discloses certain 3′-fluoro-dihydropyrimidine nucleosides. WO 92/08727 discloses β-L-2′-deoxy-3′-fluoro-5-substituted uridine nucleosides for the treatment of herpes simplex 1 and 2.

European Patent Publication No. 0 352 248 discloses a broad genus of L-ribofuranosyl purine nucleosides for the treatment of HIV, herpes, and hepatitis. While certain 2′-fluorinated purine nucleosides fall within the broad genus, there is no information given in the specification on how to make these compounds in the specification, and they are not among specifically disclosed or the preferred list of nucleosides in the specification. The specification does disclose how to make 3′-ribofuranosyl fluorinated nucleosides. A similar specification is found in WO 88/09001, filed by Aktiebolaget Astra.

European Patent Publication No. 0 357 571 discloses a broad group of β-D and α-D pyrimidine nucleosides for the treatment of AIDS which among the broad class generically includes nucleosides that can be substituted in the 2′ or 3′-position with a fluorine group. Among this broad class, however, there is no specific disclosure of 2′-fluorinated nucleosides or a method for their production.

European Patent Publication No. 0 463 470 discloses a process for the preparation of (5S)-3-fluoro-tetrahydro-5-[(hydroxy)methyl]-2-(3H)-furanone, a known intermediate in the manufacture of 2′-fluoro-2′,3′-dideoxynucleosides such as 2′-fluoro-2′,3′-dideoxycytidine.

U.S. Pat. Nos. 5,817,799 and 5,336,764 disclose β-D-2′-fluoroarabinofuranosyl nucleosides, and a method for their production, which are intermediates in the synthesis of 2′,3′-dideoxy-2′-fluoroarabinosyl nucleosides.

U.S. Pat. No. 4,625,020 discloses a method of producing 1-halo-2-deoxy-2-fluoroarabinofuranosyl derivatives bearing protective ester groups from 1,3,5-tri-O-acylribofuranose.

U.S. Pat. No. 6,348,587 and International Publication No. WO 99/43691 disclose certain 2′-fluoronucleosides, including certain 2′-fluoro-2′,3′-dideoxy-2′,3′-didehydro-4′-((S, CH, or CHF))-nucleosides, and their uses for the treatment of HIV, hepatitis (B or C), or proliferative conditions.

International Publication Nos. WO 01/90121 and WO 01/92282 disclose a wide variety of nucleosides for the treatment of HCV and flaviviruses and pestiviruses, respectively, including certain 2′-halo-2′,3′-dideoxy-2′,3′-didehydro-4′-(O, S, SO₂ or CH₂)-nucleosides.

International Publication Nos. WO 04/02422, WO 04/02999, and WO 04/03000 disclose 2′-C-methyl ribonucleosides for the treatment of HCV and flaviviruses and pestiviruses.

International Publication No. WO 05/003147 discloses 2′-deoxy-2′-fluoro-2′-methyl ribonucleosides for the treatment of HCV and flaviviruses and pestiviruses, respectively.

A nucleoside 5′-phosphonate is essentially a nucleoside monophosphate analogue. However, a phosphonate has the advantage over its phosphate counterpart of being metabolically stable, as its phosphorus-carbon bond is not susceptible to phosphatase hydrolysis. More importantly, the presence of a 5′-phosphonate allows the first phosphorylation step required for nucleoside activation to be skipped, therefore bypassing this inefficient and often rate-limiting step in the conversion to 5′-triphosphate. Like a nucleoside monophosphate, a nucleoside phosphonate can be further phosphorylated by cellular nucleotide kinases. The concept of nucleoside phosphonate has been applied to design chain terminators for anti-HIV chemotherapy and proved to be valid. 9-(2-Phosphonylmethoxypropyl)adenine (PMPA) and 9-(2-phosphonylmethoxyethyl)adenine (PMEA) are two effective and potent nucleoside phosphonate chain terminators for HIV reverse transcriptase (RT). See De Clercq, et al., A novel selective broad-spectrum anti-DNA virus agent, Nature 1986, 323, 464-467; Balzarini, et al., Marked in vivo anti-retrovirus activity of 9-(2-phosphonylmethoxyethyl)-adenine, a selective anti-human immunodeficiency virus agent, Proc. Natl. Acad. Sci. U.S.A. 1989, 86, 332-336; Balzarini, et al., Differential anti-herpes virus and anti-retrovirus effects of the (S) and (R) enantiomers of acyclic nucleoside phosphonates: Potent and selective in vitro and in vivo anti-retrovirus activities of (R)-9-(2-phosphonomethoxypropyl)-2,6-diaminopurine, Antimicrob. Agents Chemother. 1993, 37, 332-338; De Clercq, E. The acyclic nucleoside phosphonates from inception to clinical use: historical perspective, Antiviral Research, 2007, 75, 1-13.

European Patent No. 0398231, U.S. Pat. No. 5,886,179, and International Publication No. WO 04/096233 disclose a wide variety of nucleoside phosphonates, and their uses as anti-tumor and antiviral agents.

U.S. Patent Publication Nos. 05/2155513, 04/023921, and International Publication Nos. WO 04/096286, WO 04/096235 disclose a wide variety of nucleoside phosphonates, and their uses for the treatment of HIV, hepatitis (B or C), or proliferative conditions.

European Patent Publication No. 0369409 discloses certain carbocyclic nucleoside phosphonates, including ribo, deoxyribo, and halogen (excluding fluorine) substituted carbocyclic nucleoside phosphonates, and their uses for the treatment of tumor and viral infections.

Hoh, Y. et al reported certain 5′-phosphonates can inhibit the HCV replication with moderate activity, see also “Design, synthesis, and antiviral activity of adenosine 5′-phosphonate analogues as chain terminators against hepatitis C virus”, J. Med. Chem. 2005, 48, 2867-2875.

U.S. Patent Publication No. 07/022,5249 discloses 2′-fluoronucleoside phosphonates, and their uses for the treatment of HIV, hepatitis (B or C), or proliferative conditions.

International Publication No. WO 08/005,242 discloses a wide variety of nucleoside 5′-phosphinate analogues as antiviral agents.

In designing new phosphonates, it has been suggested that α-fluorination might lead to better mimic natural phosphates, see reviews by Berowitz, D. B. and Bose, M. “(α-Monofluoroalkyl)phosphonates: a class of isoacidic and “tunable” mimics of biological phosphates”, J. Fluorine Chem. 2001, 112, 13-33; and Romanenko, V. D. and Kukhar, V. P.” Chem. Rev. 2006, 106, 3868.

In light of the fact that acquired immune deficiency syndrome, AIDS-related complex, hepatitis B virus and hepatitis C virus have reached epidemic levels worldwide, and have tragic effects on the infected patient, there remains a strong need to provide new effective pharmaceutical agents to treat these diseases that have low toxicity to the host. Further, there is a need to provide new antiproliferative agents.

Therefore, it is an object of the present invention to provide a method and composition for the treatment of human patients or other host animals infected with HIV.

It is another object of the present invention to provide a method and composition for the treatment of human patients infected with hepatitis B or C.

It is a further object of the present invention to provide new antiproliferative agents.

It is still another object of the present invention to provide a new process for the preparation of 2′-fluoronucleoside phosphonates of the present invention.

### SUMMARY OF THE INVENTION

The present invention includes β-D and β-L-nucleoside phosphonate derivatives, pharmaceutical compositions comprising such compounds, as well as methods to treat or prevent an HIV infection, HBV infection, HCV infection or abnormal cellular proliferation comprising administering said compounds or compositions. In addition, the present invention includes the process for the preparation of such compounds, and the related β-D and β-L-nucleoside phosphonate derivatives.

In its principal embodiment, the compound of the invention is a 2′-fluoronucleoside phosphonate of the general formula (I) or (II):

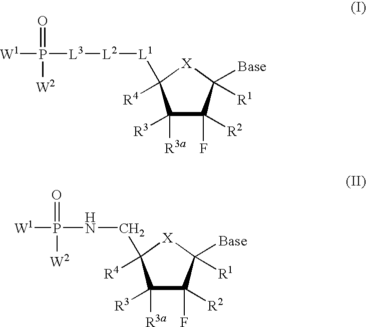

or a pharmaceutically acceptable salt, ester, stereoisomer, tautomer, solvate, prodrug, or combination thereof, wherein:

### X is O, S, SO₂, or CH₂;

L¹ at each occurrence is —CR¹⁰R¹¹—, and L² at each occurrence is —CR¹²R¹³—, wherein one of R¹⁰, R¹¹, R¹², and R¹³ is a halogen or hydroxyl and the rest are selected from a group consisting of: hydrogen, deuterium, hydroxyl, or halogen; or R¹⁰ and R¹¹ or R¹² and R¹³ taken together with the carbon atom to which they are attached form a carbonyl or C₂-C₈ alkenylene group; or R¹⁰ and R¹² or R¹¹ and R¹³ taken together form a single bond; or R¹⁰ and R¹² or R¹¹ and R¹³ taken together with the carbon atom to which they are attached form a cyclopropane or oxirane ring;
L³ at each occurrence is each independently —CR¹⁰R¹¹— or absent;
R¹, R² and R⁴ at each occurrence are each independently selected from the group consisting of:
1) —C₁-C₈ alkyl, —C₂-C₈ alkenyl, —C₂-C₈ alkynyl or —C₃-C₈ cycloalkyl each containing 0, 1, 2, or 3 heteroatoms selected from O, S or N;
2) substituted —C₁-C₈ alkyl, substituted —C₂-C₈ alkenyl, substituted —C₂-C₈ alkynyl or substituted —C₃-C₈ cycloalkyl each containing 0, 1, 2, or 3 heteroatoms selected from O, S or N;
3) hydrogen;
4) deuterium;
5) —CN; and
6) halogen;
   R³ and R^{3a} at each occurrence are each independently selected from the group consisting of:
1) hydrogen;
2) deuterium;
3) hydroxyl or protected hydroxyl;
4) halogen;
5) —CN;
6) —N₃;
7) —NR¹⁴R¹⁵, wherein R¹⁴ and R¹⁵ at each occurrence are each independently selected from the group consisting of: hydrogen and substituted or unsubstituted —C₁-C₈ alkyl;
8) —C₁-C₈ alkyl, —C₂-C₈ alkenyl, —C₂-C₈ alkynyl or —C₃-C₈ cycloalkyl each containing 0, 1, 2, or 3 heteroatoms selected from O, S or N; and
9) substituted —C₁-C₈ alkyl, substituted —C₂-C₈ alkenyl, substituted —C₂-C₈ alkynyl or substituted —C₃-C₈ cycloalkyl each containing 0, 1, 2, or 3 heteroatoms selected from O, S or N;
   or R³ and R^{3a} taken together with the carbon atom to which they are attached form a group consisting of:
1) C═O;
2) C═NR¹⁴;
3) C═CR¹⁴R¹⁵;
4) C₃-C₈ cycloalkyl; and
5) 3-7 membered heterocyclic ring wherein containing at least one heteroatom from O, S or N;
   W¹ and W² at each occurrence are each independently a group of the formula:

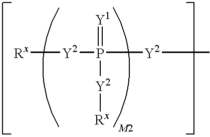

wherein:
Y¹ is each independently O, S, NR, ⁺N(O)(R), N(OR), ⁺N(O)(OR), or N—NR₂; wherein R is independently hydrogen, halogen, C₁-C₈ alkyl, substituted C₁-C₈ alkyl, C₂-C₈ alkenyl, substituted C₂-C₈ alkenyl, C₂-C₈ alkynyl, substituted C₂-C₈ alkynyl, aryl, substituted aryl, heterocyclic, substituted heterocyclic or a protecting group;
Y² is each independently a bond, O, CR₂, NR, ⁺N(O)(R), N(OR), ⁺N(O)(OR), N—NR₂, S, S—S, S(O), or S(O)₂;
M2 is 0, 1 or 2;
R^{x} is each independently R^{y}, a protecting group, or the formula:

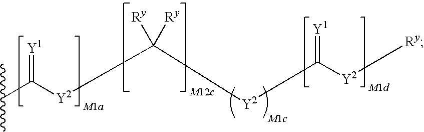

wherein:
M1a, M1c, and M1d are independently 0 or 1;
M12c is 0, 1, 2, 3, 4, 5, 6, 7, 8, 9, 10, 11 or 12;
or when taken together, two R^{x} are optionally substituted C₂-C₈ alkylene thereby forming a phosphorous-containing heterocycle;
each R^{y} is independently H, F, Cl, Br, I, OH, R, —C(═Y¹)R, —C(═Y¹)OR, —C(═Y¹)N(R)₂, —N(R)₂, —⁺N(R)₃, —SR, —S(O)R, —S(O)₂R, —S(O)(OR), —S(O)₂(OR), —OC(═Y¹)R, —OC(═Y¹)OR, —OC(═Y¹)(N(R)₂), —SC(═Y¹)R, —SC(═Y¹)OR, —SC(═Y¹)(N(R)₂), —N(R)C(═Y¹)R, —N(R)C(═Y¹)OR, or —N(R)C(═Y¹)N(R)₂, amino (—NH₂), ammonium (—NH₃⁺), alkylamino, dialkylamino, trialkylammonium, C₁-C₈ alkyl, C₁-C₈ alkyl halide, carboxylate, sulfamate, C₁-C₈ alkyl-hydroxyl, C₁-C₈ alkyl-thiol, sulfonamide (—SO₂NR₂), nitrile (—CN), azido (—N₃), nitro (—NO₂), C₁-C₈ alkoxy (—OR), a protecting group, or W³; or when taken together, two R^{y} on the same carbon atom forms a carbocyclic ring of 3-7 carbon atoms;
W³ is W⁴ or W⁵; wherein W⁴ is R, —C(Y¹)R^{y}, C(Y¹)W⁵, —SO₂R^{y}, or —SO₂W⁵; and W⁵ is a substituted or unsubstituted alicyclic, a substituted or unsubstituted aryl, or a substituted or unsubstituted heterocyclic group.
   Base is a heterocycle containing at least one nitrogen atom, preferably a pyrimidine or purine base of the general formula of (III) or (IV):

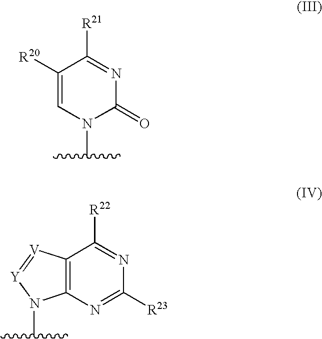

wherein:
W, Y and V are each independently N, CH, or CR¹⁶; wherein R¹⁶ is a halogen, —C₁-C₈ alkyl, ary, acyl;
R²⁰ is each independently selected from the group consisting of:
   1) —C₁-C₈ alkyl, —C₂-C₈ alkenyl, —C₂-C₈ alkynyl or —C₃-C₈ cycloalkyl each containing 0, 1, 2, or 3 heteroatoms selected from O, S or N;
   2) substituted —C₁-C₈ alkyl, substituted —C₂-C₈ alkenyl, substituted —C₂-C₈ alkynyl or substituted —C₃-C₈ cycloalkyl each containing 0, 1, 2, or 3 heteroatoms selected from O, S or N;
   3) hydrogen;
   4) deuterium;
   5) —CN;
   6) halogen; and
   7) —C(O)R¹⁷, wherein R¹⁷ is —C₁-C₈ alkyl, OH, OR¹⁴, or NR¹⁴R¹⁵; and R¹², R²² and R²³ are independently a hydrogen, halogen (F, Cl, Br, I), OH, OR¹⁴, SH, SR¹⁴, NH₂, NHR¹⁴, NR¹⁴R¹⁵, OCOR¹⁴, OCOOR¹⁴, NHCOR¹⁴, NHCOOR¹⁴.

In another embodiment, the present invention provides a pharmaceutical composition comprising a therapeutically effective amount of a compound or combination of compounds of the present invention, or a pharmaceutically acceptable salt form, prodrug, salt of a prodrug, stereoisomer, tautomer, solvate, or combination thereof, in combination with a pharmaceutically acceptable carrier or excipient.

In yet another embodiment, the present invention provides a method of inhibiting the replication of an RNA or DNA containing virus comprising contacting said virus with a therapeutically effective amount of a compound or a combination of compounds of the present invention, or a pharmaceutically acceptable salt, prodrug, salt of a pro drug, stereoisomer, tautomer, solvate, or combination thereof. Particularly, this invention is directed to methods of inhibiting the replication of HIV, HBV and HCV.

In still another embodiment, the present invention provides a method of treating or preventing infection caused by an RNA or DNA-containing virus comprising administering to a patient in need of such treatment a therapeutically effective amount of a compound or combination of compounds of the present invention, or a pharmaceutically acceptable salt form, prodrug, salt of a prodrug, stereoisomer, or tautomer, solvate, or combination thereof. Particularly, this invention is directed to methods of treating or preventing infection caused by HIV, HBV and HCV.

Yet another embodiment of the present invention provides the use of a compound or combination of compounds of the present invention, or a therapeutically acceptable salt form, prodrug, salt of a prodrug, stereoisomer or tautomer, solvate, or combination thereof, as defined hereinafter, in the preparation of a medicament for the treatment or prevention of infection caused by RNA or DNA-containing virus, specifically HIV, HBV and HCV.

### DETAILED DESCRIPTION OF THE INVENTION

In a first embodiment of the present invention is a compound of Formula (I) or (II) as illustrated above, or a pharmaceutically acceptable salt, ester or prodrug thereof.

In a second embodiment of the present invention is a β-D 2′-fluoronucleoside phosphonate represented by formula (I) or (II) as illustrated above, or its β-L enantiomer, or pharmaceutically acceptable salt or prodrug thereof.

In a particular embodiment of the present invention is a β-D 2′-fluoronucleoside phosphonate represented by formula (Ia), or its β-L enantiomer, or pharmaceutically acceptable salt or prodrug thereof:

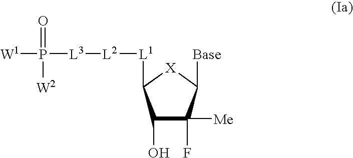

wherein L¹, L², L³, W¹, W², X and Base are as previously defined.

In another particular embodiment of the present invention is a β-D nucleoside phosphonate represented by formula (Ib), or its β-L enantiomer, or pharmaceutically acceptable salt or prodrug thereof:

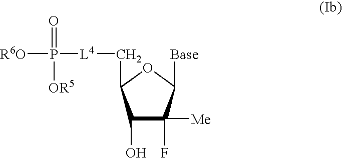

wherein L⁴ is —CHF— or —CF₂—, and Base are as previously defined; R⁵ and R⁶ are independently a hydrogen, phosphate, diphosphate, or a group that is preferentially removed in a hepatocyte to yield the corresponding OH group. The term “preferentially removed in a hepatocyte” as used herein means at least part of the group is removed in a hepatocyte at a rate higher than the rate of removal of the same group in a non-hepatocytic cell (e.g., fibroblast or lymphocyte). It is therefore contemplated that the removable group includes all pharmaceutically acceptable groups that can be removed by a reductase, esterase, cytochrome P450 or any other specific liver enzyme. Alternative contemplated groups may also include groups that are not necessarily preferentially removed in a hepatocyte, but effect at least some accumulation and/or specific delivery to a hepatocyte (e.g., esters with selected amino acids, including valine, leucine, isoleucine, or polyarginine or polyaspartate).

In another particular embodiment of the present invention is a β-D nucleoside phosphonate represented by formula (Ic), or its β-L enantiomer, or pharmaceutically acceptable salt or prodrug thereof:

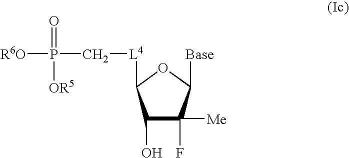

wherein L⁴, R⁵, R⁶ and Base are as previously defined.

In yet another particular embodiment of the present invention is a β-D nucleoside phosphonate represented by formula (Id), or its β-L enantiomer, or pharmaceutically acceptable salt or prodrug thereof:

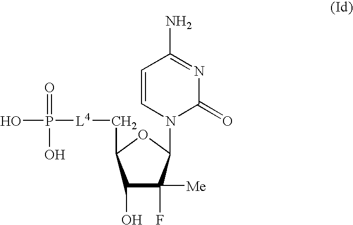

wherein L⁴ is as previously defined.

In another particular embodiment of the present invention is a β-D nucleoside phosphonate represented by formula (Ie), or its β-L enantiomer, or pharmaceutically acceptable salt or prodrug thereof:

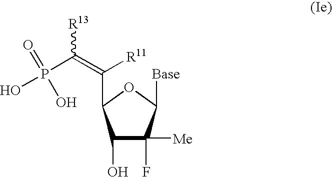

wherein R¹¹, R¹³ and Base are as previously defined.

In another particular embodiment of the present invention is a β-D nucleoside phosphonate represented by formula (If), or its β-L enantiomer, or pharmaceutically acceptable salt or prodrug thereof:

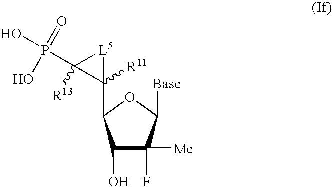

wherein L⁵ is —O— or —CH₂—, R¹¹, R¹³ and Base are as previously defined.

Embodiments of

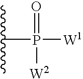

Formula (I) or (II) compounds include substructures such as:

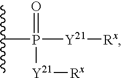

wherein Y²¹ is O or N(R); R and R^{x} are as previously defined; or

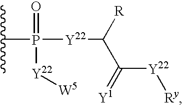

wherein Y²² is O, S or N(R); R, Y¹, W⁵ and R^{x} are as previously defined; or

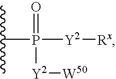

wherein W⁵⁰ is a substituted or unsubstituted aryl such as phenyl or substituted phenyl; Y² and R^{x} are as previously defined; such a substructure includes:

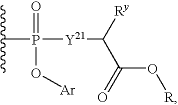

wherein Y²¹, R and R^{y} are as previously defined; or

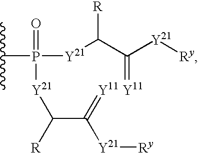

wherein Y¹¹ is O or S; each Y²¹, R and R^{y} are as previously defined.

In a particular embodiment of the present invention is a β-D 2′-fluoronucleoside phosphoramidate derivative represented by formula (IIa), or its β-L enantiomer, or pharmaceutically acceptable salt or prodrug thereof:

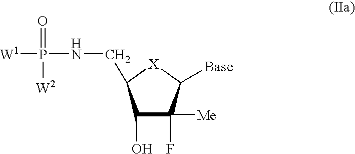

wherein W¹, W², X and Base are as previously defined.

Representative compounds of the present invention are those selected from:

1. Compound of Formula (I), wherein Base is N⁴-benzoylcytosin-1-yl, X is O, L² is CF₂, L¹ is CH₂, L³ is absent, R¹═R³═R⁴═H, R²=Me, R^{3a}═OAc, W¹═W²═OEt.
2. Compound of Formula (I), wherein Base is cytosine-1-yl, X is O, L² is CF₂, L¹ is CH₂, L³ is absent, R¹═R³═R⁴═H, R²=Me, R^{3a}═OH, W¹═W²═OEt.
3. Compound of Formula (I), wherein Base is cytosine-1-yl, X is O, L² is CF₂, L¹ is CH₂, L³ is absent, R¹═R³═R⁴═H, R²=Me, R^{3a}═OH, W¹═W²═OH.
4. Compound of Formula (I), wherein Base is uracil-1-yl, X is O, L² is CF₂, L¹ is CH₂, L³ is absent, R¹═R³═R⁴═H, R²=Me, R^{3a}═OAc, W¹═W²═OEt.
5. Compound of Formula (I), wherein Base is uracil-1-yl, X is O, L² is CF₂, L¹ is CH₂, L³ is absent, R¹═R³═R⁴═H, R²=Me, R^{3a}═OH, W¹═W²═OEt.
6. Compound of Formula (I), wherein Base is uracil-1-yl, X is O, L² is CF₂, L¹ is CH₂, L³ is absent, R¹═R³═R⁴═H, R²=Me, R^{3a}═OH, W¹═W²═OH.
7. Compound of Formula (I), wherein Base is uracil-1-yl, X is O, L² is CF₂, L¹ is CH₂, L³ is absent, R¹═R³═R⁴═H, R²=Me, R^{3a}═OC(O)CH₂CH₂C(O)Me, W¹═W²═OH.
8. Compound of Formula (I), wherein Base is uracil-1-yl, X is O, L² is CF₂, L¹ is CH₂, L³ is absent, R¹═R³═R⁴═H, R²=Me, R^{3a}═OC(O)CH₂CH₂C(O)Me, W¹═OPh, W²═OH.
9. Compound of Formula (I), wherein Base is uracil-1-yl, X is O, L² is CF₂, L¹ is CH₂, L³ is absent, R¹═R³═R⁴═H, R²=Me, R^{3a}═OC(O)CH₂CH₂C(O)Me, W¹═OPh, W²=(S)—NH—CH(Me)CO₂Me.
10. Compound of Formula (I), wherein Base is uracil-1-yl, X is O, L² is CF₂, L¹ is CH₂, L³ is absent, R¹═R³═R⁴═H, R²=Me, R^{3a}═OH, W¹═OPh, W²═(S)—NH—CH(Me)CO₂Me.
11. Compound of Formula (I), wherein Base is N⁴-levulinoylcytosin-1-yl, X is O, L² is CF₂, L¹ is CH₂, L³ is absent, R¹═R³═R⁴═H, R²=Me, R^{3a}═OC(O)CH₂CH₂C(O)Me, W¹═W²═OH.
12. Compound of Formula (I), wherein Base is N⁴-levulinoylcytosin-1-yl, X is O, L² is CF₂, L¹ is CH₂, L³ is absent, R¹═R³═R⁴═H, R²=Me, R^{3a}═OC(O)CH₂CH₂C(O)Me, W¹═OPh, W²═OH.
13. Compound of Formula (I), wherein Base is N⁴-levulinoylcytosin-1-yl, X is O, L² is CF₂, L¹ is CH₂, L³ is absent, R¹═R³═R⁴═H, R²=Me, R^{3a}═OC(O)CH₂CH₂C(O)Me, W¹═OPh, W²═(S)—NH—CH(Me)CO₂Me.
14. Compound of Formula (I), wherein Base is cytosin-1-yl, X is O, L² is CF₂, L¹ is CH₂, L³ is absent, R¹═R³═R⁴═H, R²=Me, R^{3a}═OH, W¹═OPh, W²═(S)—NH—CH(Me)CO₂Me.
15. Compound of Formula (I), wherein Base is N⁴-benzoylcytosin-1-yl, X is O, L² is CHF, L¹ is CH₂, L³ is absent, R¹═R³═R⁴═H, R²=Me, R^{3a}═OBz, W¹═W²═OEt.
16. Compound of Formula (I), wherein Base is cytosin-1-yl, X is O, L² is CHF, L¹ is CH₂, L³ is absent, R¹═R³═R⁴═H, R²=Me, R^{3a}═OH, W¹═W²═OEt.
17. Compound of Formula (I), wherein Base is cytosin-1-yl, X is O, L² is CHF, L¹ is CH₂, L³ is absent, R¹═R³═R⁴═H, R²=Me, R^{3a}═OH, W¹═W²═OH.
18. Compound of Formula (I), wherein Base is uracil-1-yl, X is O, L² is CHF, L¹ is CH₂, L³ is absent, R¹═R³═R⁴═H, R²=Me, R^{3a}═OBz, W¹═W²═OEt.
19. Compound of Formula (I), wherein Base is uracil-1-yl, X is O, L² is CHF, L¹ is CH₂, L³ is absent, R¹═R³═R⁴═H, R²=Me, R^{3a}═OH, W¹═W²═OEt.
20. Compound of Formula (I), wherein Base is uracil-1-yl, X is O, L² is CHF, L¹ is CH₂, L³ is absent, R¹═R³═R⁴═H, R²=Me, R^{3a}═OH, W¹═W²═OH.
21. Compound of Formula (I), wherein Base is N⁴-benzoylcytosin-1-yl, X is O, L² is CHF, L¹ is CH₂, L³ is absent, R¹═R³═R⁴═H, R²=Me, R^{3a}═OAc, W¹═W²═OEt.
22. Compound of Formula (I), wherein Base is uracil-1-yl, X is O, L² is CHF, L¹ is CH₂, L³ is absent, R¹═R³═R⁴═H, R²=Me, R^{3a}═OBz, W¹═W²═OEt.
23. Compound of Formula (I), wherein Base is uracil-1-yl, X is O, L² is CHF, L¹ is CH₂, L³ is absent, R¹═R³═R⁴═H, R²=Me, R^{3a}═OC(O)CH₂CH₂C(O)Me, W¹═W²═OEt.
24. Compound of Formula (I), wherein Base is uracil-1-yl, X is O, L² is CHF, L¹ is CH₂, L³ is absent, R¹═R³═R⁴═H, R²=Me, R^{3a}═OC(O)CH₂CH₂C(O)Me, W¹═W²═OH.
25. Compound of Formula (I), wherein Base is uracil-1-yl, X is O, L² is CHF, L¹ is CH₂, L³ is absent, R¹═R³═R⁴═H, R²=Me, R^{3a}═OC(O)CH₂CH₂C(O)Me, W¹═OPh, W²═OH.
26. Compound of Formula (I), wherein Base is uracil-1-yl, X is O, L² is CHF, L¹ is CH₂, L³ is absent, R¹═R³═R⁴═H, R²=Me, R^{3a}═OC(O)CH₂CH₂C(O)Me, W¹═OPh, W²═(S)—NH—CH(Me)CO₂Me.
27. Compound of Formula (I), wherein Base is uracil-1-yl, X is O, L² is CHF, L¹ is CH₂, L³ is absent, R¹═R³═R⁴═H, R²=Me, R^{3a}═OH, W¹═OPh, W²═(S)—NH—CH(Me)CO₂Me.
28. Compound of Formula (I), wherein Base is N⁴-cytosin-1-yl, X is O, L² is CHF, L¹ is CH₂, L³ is absent, R¹═R³═R⁴═H, R²=Me, R^{3a}═OC(O)CH₂CH₂C(O)Me, W¹═W²═OEt.
29. Compound of Formula (I), wherein Base is N⁴-cytosin-1-yl, X is O, L² is CHF, L¹ is CH₂, L³ is absent, R¹═R³═R⁴═H, R²=Me, R^{3a}═OC(O)CH₂CH₂C(O)Me, W¹═W²═OH.
30. Compound of Formula (I), wherein Base is N⁴-cytosin-1-yl, X is O, L² is CHF, L¹ is CH₂, L³ is absent, R¹═R³═R⁴═H, R²=Me, R^{3a}═OC(O)CH₂CH₂C(O)Me, W¹═OPh, W²═OH.
31. Compound of Formula (I), wherein Base is N⁴-cytosin-1-yl, X is O, L² is CHF, L¹ is CH₂, L³ is absent, R¹═R³═R⁴═H, R²=Me, R^{3a}═OC(O)CH₂CH₂C(O)Me, W¹═OPh, W²═(S)—NH—CH(Me)CO₂Me.
32. Compound of Formula (I), wherein Base is N⁴-cytosin-1-yl, X is O, L² is CHF, L¹ is CH₂, L³ is absent, R¹═R³═R⁴═H, R²=Me, R^{3a}═OH, W¹═OPh, W²═(S)—NH—CH(Me)CO₂Me.
33. Compound of Formula (II), wherein Base is N⁴-cytosin-1-yl, X is O, R¹═R³═R⁴═H, R²=Me, R^{3a}═OBz, W¹═W²═OMe.
34. Compound of Formula (II), wherein Base is N⁴-cytosin-1-yl, X is O, R¹═R³═R⁴═H, R²=Me, R^{3a}═OH, W¹═OH, W²═OMe.

In one embodiment of the invention, the 2′-fluoronucleoside phosphonate derivatives of the invention are the isolated β-D or β-L isomer. In another embodiment of the invention, the nucleoside phosphonate derivatives are enantiomerically enriched. In yet another embodiment of the invention, the nucleoside phosphonate derivative is in a enantiomeric mixture in which the desired enantiomer is at least 95%, 98% or 99% free of its enantiomer. In a preferred embodiment, the nucleoside is enantiomerically enriched.

In one embodiment of the present invention, the compounds of the formula (I) are in the β-D configuration. In an alternate embodiment of the present invention, the compounds of formula (I) are in the β-L configuration.

The nucleoside phosphonate derivatives depicted above are in the β-D configuration, however, it should be understood that the nucleoside phosphonate derivatives can be either in the β-L or β-D configuration.

The nucleoside phosphonate derivatives of the present invention are biologically active molecules that are useful in the treatment or prophylaxis of viral infections, and in particular human immunodeficiency virus (HIV) and/or hepatitis B virus (HBV) infection. The compounds are also useful for the treatment of abnormal cellular proliferation, including tumors and cancer. In another embodiment of the present invention, any of the active compounds are useful in the treatment of HCV. One can easily determine the spectrum of activity by evaluating the compound in the assays described herein or with another confirmatory assay.

For instance, in one embodiment the efficacy of the antiviral compound is measured according to the concentration of compound necessary to reduce the plaque number of the virus in vitro, according to methods set forth more particularly herein, by 50% (i.e. the compound's EC₅₀). In preferred embodiments the compound exhibits an EC₅₀ of less than 15 or preferably, less than 10 micromolar in vitro.

In another embodiment, for the treatment or prophylaxis of a viral infection, and in particular an HIV, HCV or HBV infection, in a host, the active compound or its derivative or salt can be administered in combination or alternation with another antiviral agent, such as an anti-HIV agent or anti-hepatitis agent, including those of the formula above. Alternatively, for the treatment of abnormal cellular proliferation, such as tumors and cancer, in a host, the active compound or its derivative or salt can be administered in combination or alternation with another antiproliferative agent, such as an anti-neoplastic agent, including those of the formula above. In general, in combination therapy, effective dosages of two or more agents are administered together, whereas during alternation therapy, an effective dosage of each agent is administered serially. The dosages will depend on absorption, inactivation and excretion rates of the drug as well as other factors known to those of skill in the art. It is to be noted that dosage values will also vary with the severity of the condition to be alleviated. It is to be further understood that for any particular subject, specific dosage regimens and schedules should be adjusted over time according to the individual need and the professional judgment of the person administering or supervising the administration of the compositions.

Nonlimiting examples of antiviral agents that can be used in combination with the compounds disclosed herein include 2-hydroxymethyl-5-(5-fluorocytosin-1-yl)-1,3-oxathiolane (FTC); the (−)-enantiomer of 2-hydroxymethyl-5-(cytosin-1-yl)-1,3-oxathiolane (3TC); carbovir, acyclovir, interferon, famciclovir, penciclovir, AZT, DDI, DDC, D4T, abacavir, L-(−)-FMAU, L-dT, -D-2′-C-methylcytidine, L-DDA phosphate prodrugs, and β-D-dioxolane nucleosides such as β-D-dioxolanyl-guanine (DG), β-D-dioxolanyl-2,6-diaminopurine (DAPD), and β-D-dioxolanyl-6-chloropurine (ACP), non-nucleoside reverse transcriptase inhibitors such as nevirapine, MKC-442, DMP-266 (sustiva) and also protease inhibitors such as ritonavir, indinavir, saquinavir, DMP-450 and others.

The compounds of the present invention can also be used to treat equine infectious anemia virus (EIAV), feline immunodeficiency virus, and simian immunodeficiency virus. (Wang, S., et al, “Activity of nucleoside and non-nucleoside reverse transcriptase inhibitors (NNRTI) against equine infectious ane mia virus (EIAV).” First National Conference on Human Retroviruses and Related Infections, Washington, D.C., Dec. 12-16, 1993; Sellon D. C., “Equine Infectious Anemia” Vet. Clin. North Am. Equine Pract. United States, 9: 321-336, 1993; Philpott, M. S., et al “Evaluation of 9-(2-phosphonylmethoxyethyl)adenine therapy for feline immunodeficiency virus using a quantitative polymerase chain reaction” Vet. Immunol Immunopathol. 35:155166, 1992.

The present invention also provides a pharmaceutical composition for the treatment and/or prophylaxis of a viral infection, and in particular a HBV, HCV or HIV infection, in a host, preferably a human, comprising a therapeutically effective amount of an active compound of the present invention, optionally in a pharmaceutically acceptable carrier.

The present invention also provides a pharmaceutical composition for the treatment and/or prophylaxis of an abnormal cellular proliferation, such as tumors and cancer, in a host, preferably a human, comprising a therapeutically effective amount of an active compound of the present invention, optionally in a pharmaceutically acceptable carrier.

The present invention also provides a pharmaceutical composition for the treatment and/or prophylaxis of a viral infection, and in particular a HBV, HCV or HIV infection, in a host, preferably a human, comprising a therapeutically effective amount of an active compound of the present invention, in combination with one or more other effective antiviral agent, and in particular an anti-HBV, anti-HCV or anti-HIV agent, optionally in a pharmaceutically acceptable carrier.

The present invention also provides a pharmaceutical composition for the treatment and/or prophylaxis of an abnormal cellular proliferation, such as tumors and cancer, in a host, preferably a human, comprising a therapeutically effective amount of an active compound of the present invention, in combination with one or more other effective antinroliferative agent, such as an antineoplastic agent, optionally in a pharmaceutically acceptable carrier.

The present invention also provides a method for the treatment and/or prophylaxis of a viral infection, and in particular a HBV, HCV or HIV infection, in a host, preferably a human, comprising administering to the host a therapeutically effective amount of an active compound of the present invention, optionally in a pharmaceutically acceptable carrier.

The present invention also provides a method for the treatment and/or prophylaxis of an abnormal cellular proliferation, such as tumors and cancer, in a host, preferably a human, comprising administering to the host a therapeutically effective amount of an active compound of the present invention, optionally in a pharmaceutically acceptable carrier.

The present invention also provides a method for the treatment and/or prophylaxis of a viral infection, and in particular a HBV, HCV or HIV infection, in a host, preferably a human, comprising administering to the host a therapeutically effective amount of an active compound of the present invention, in combination and/or alternation with one or more other effective antiviral agent, and in particular an anti-HBV, anti-HCV or anti-HIV agent, optionally in a pharmaceutically acceptable carrier.

The present invention also provides a method for the treatment and/or prophylaxis of an abnormal cellular proliferation, such as tumors and cancer, in a host, preferably a human, comprising administering to the host a therapeutically effective amount of an active compound of the present invention, in combination andor alternation with one or more other effective antiproliferative agent, such as an antineoplastic agent, optionally in a pharmaceutically acceptable carrier.

The present invention also provides a use of an active compound of the present invention, optionally in a pharmaceutically acceptable carrier, for the treatment and/or prophylaxis of a viral infection, and in particular a HBV, HCV or HIV infection, in a host, preferably a human.

The present invention also provides a use of an active compound of the present invention, optionally in a pharmaceutically acceptable carrier, for the treatment and/or prophylaxis of an abnormal cellular proliferation, such as tumors and cancer, in a host, preferably a human.

The present invention also provides a use of an active compound of the present invention, in combination andor alternation with one or more other effective antiviral agent, and in particular an anti-HBV, anti-HCV or anti-HIV agent, optionally in a pharmaceutically acceptable carrier, for the treatment andor prophylaxis of a viral infection, and in particular a HBV, HCV or HIV infection, in a host, preferably a human.

The present invention also provides a use of an active compound of the present invention, in combination and/or alternation with one or more other effective antiproliferative agent, such as an antineoplastic agent, optionally in a pharmaceutically acceptable carrier, for the treatment and/or prophylaxis of an abnormal cellular proliferation, such as tumors and cancer, in a host, preferably a human.

The present invention also provides a use of an active compound of the present invention, optionally in a pharmaceutically acceptable carrier, in the manufacture of a medicament for the treatment and/or prophylaxis of a viral infection, and in particular a HBV, HCV or HIV infection, in a host, preferably a human.

The present invention also provides a use of an active compound of the present invention, optionally in a pharmaceutically acceptable carrier, in the manufacture of a medicament for the treatment and/or prophylaxis of an abnormal cellular proliferation, such as tumors and cancer, in a host, preferably a human.

The present invention also provides a use of an active compound of the present invention, in combination andor alternation with one or more other effective antiviral agent, and in particular an anti-HBV, anti-HCV or anti-HIV agent, optionally in a pharmaceutically acceptable carrier, in the manufacture of a medicament for the treatment and/or prophylaxis of a viral infection, and in particular a HBV, HCV or HIV infection, in a host, preferably a human.

The present invention also provides a use of an active compound of the present invention, in combination and/or alternation with one or more other effective antiviral agent, and in particular an anti-HCV agent, optionally in a pharmaceutically acceptable carrier, in the manufacture of a medicament for the treatment andor prophylaxis of a viral infection, and in particular a HCV infection, in a host, preferably a human.

The present invention also provides a use of an active compound of the present invention, in combination and/or alternation with one or more other effective antiproliferative agent, such as an antineoplastic agent, optionally in a pharmaceutically acceptable carrier, in the manufacture of a medicament for the treatment and/or prophylaxis of an abnormal cellular proliferation, such as tumors and cancer, in a host, preferably a human.

The invention also provides synthetic methods useful for preparing the compounds of the invention, as well as intermediates disclosed herein that are useful in the preparation of the compounds of the present invention.

The invention as disclosed herein provides methods and compositions for the treatment of HIV, hepatitis B or C, or abnormal cellular proliferation, in humans or other host animals, that includes administering a therapeutically effective amount of a β-D- or β-L-nucleoside phosphonate derivative, a pharmaceutically acceptable derivative, including a compound which has been alkylated or acylated on a sugar or phosphonate moiety, or on the purine or pyrimidine, or a pharmaceutically acceptable salt thereof, optionally in a pharmaceutically acceptable carrier. The compounds of this invention either possess antiviral (i.e., anti-HIV-1, anti-HIV-2, anti-hepatitis B/C virus) activity or antiproliferative activity, or are metabolized to a compound that exhibits such activity. The invention as disclosed herein also includes the process for the preparation of such β-D- or β-L-nucleoside phosphonate derivatives.

In summary, the present invention includes the following features:

(a) β-L and β-D-nucleoside phosphonate derivatives, as described herein, and pharmaceutically acceptable derivatives and salts thereof;

(b) synthesis of the β-L and β-D-nucleoside phosphonate derivatives as described herein, and pharmaceutically acceptable derivatives and salts thereof,

(c) β-L and β-D-nucleoside phosphonate derivatives as described herein, and pharmaceutically acceptable derivatives and salts thereof for use in medical therapy, for example for the treatment or prophylaxis of an HIV, hepatitis B (or C) virus infection or for the treatment of abnormal cellular proliferation;

(d) pharmaceutical formulations comprising a β-D or β-L-nucleoside phosphonate derivative described herein, or a pharmaceutically acceptable derivative or salt thereof, together with a pharmaceutically acceptable carrier or diluent;

(e) pharmaceutical formulations comprising a β-D or β-L-nucleoside phosphonate derivative described herein, or a pharmaceutically acceptable derivative or salt thereof, together with another active ingredient, such as another antiviral agent or antiproliferative agent;

(f) methods to treat a host suffering from an HIV infection, hepatitis B virus infection, hepatitis C virus infection or abnormal cellular proliferation, comprising administering a therapeutically effective amount of a β-D or β-L-nucleoside phosphonate derivative described herein, or a pharmaceutically acceptable derivative or salt thereof,

(g) methods to treat a host suffering from an HIV infection, hepatitis C virus infection, hepatitis B virus infection or abnormal cellular proliferation, comprising administering a therapeutically effective amount of a β-D or β-L-nucleoside phosphonate derivative described herein, or a pharmaceutically acceptable derivative or salt thereof, in combination or alternation with another active ingredient, such as another antiviral agent or antiproliferative agent;

(h) use of a β-D or β-L-nucleoside phosphonate derivative described herein, or a pharmaceutically acceptable derivative or salt thereof, in medical therapy, for example for the treatment or prophylaxis of HIV infection, hepatitis C virus infection, hepatitis B virus infection or an abnormal cellular proliferation;

(i) use of a β-D or β-L-nucleoside phosphonate derivative described herein, or a pharmaceutically acceptable derivative or salt thereof, as an antiviral agent;

(j) use of a β-D or β-L-nucleoside phosphonate derivative described herein, or a pharmaceutically acceptable derivative or salt thereof, as an antiproliferative agent;

(k) use of a β-D or β-L-nucleoside phosphonate derivative described herein, or a pharmaceutically acceptable derivative or salt thereof, in combination or alternation with another active ingredient, such as another antiviral agent or antiproliferative agent in medical therapy, for example for the treatment or prophylaxis of HIV infection, hepatitis C virus infection, hepatitis B virus infection or abnormal cellular proliferation;

(l) use of a β-D or β-L-nucleoside phosphonate derivative described herein, or a pharmaceutically acceptable derivative or salt thereof, for treatment or prophylaxis of HIV infection, hepatitis C virus infection, hepatitis B virus infection or abnormal cellular proliferation;

(m) use of a β-D or β-L-nucleoside phosphonate derivative described herein, or a pharmaceutically acceptable derivative or salt thereof, in the manufacture of a medicament for treatment or prophylaxis of an HIV infection, an hepatitis B virus infection or abnormal cellular proliferation.

### Stereoisomerism and Polymorphism

The compounds of the present invention may have asymmetric centers and occur as racemates, racemic mixtures, individual diastereomers or enantiomers, with all isomeric forms being included in the present invention. Compounds of the present invention having a chiral center may exist in and be isolated in optically active and racemic forms. Some compounds may exhibit polymorphism. The present invention encompasses racemic, optically-active, polymorphic, or stereoisomeric form, or mixtures thereof, of a compound of the invention, which possess the useful properties described herein. The optically active forms can be prepared by, for example, resolution of the racemic form by recrystallization techniques, by synthesis from optically active starting materials, by chiral synthesis, or by chromatographic separation using a chiral stationary phase or by enzymatic resolution.

Examples of methods to obtain optically active materials include at least the following:

i) physical separation of crystals: a technique whereby macroscopic crystals of the individual enantiomers are manually separated. This technique can be used if crystals of the separate enantiomers exist, i.e., the material is a conglomerate, and the crystals are visually distinct;

ii) simultaneous crystallization: a technique whereby the individual enantiomers are separately crystallized from a solution of the racemate, possible only if the latter is a conglomerate in the solid state;

iii) enzymatic resolutions: a technique whereby partial or complete separation of a racemate by virtue of differing rates of reaction for the enantiomers with an enzyme;

iv) enzymatic asymmetric synthesis: a synthetic technique whereby at least one step of the synthesis uses an enzymatic reaction to obtain an enantiomerically pure or enriched synthetic precursor of the desired enantiomer;

v) chemical asymmetric synthesis: a synthetic technique whereby the desired enantiomer is synthesized from an achiral precursor under conditions that produce asymmetry (i.e., chirality) in the product, which may be achieved using chiral catalysts or chiral auxiliaries;

vi) diastereomer separations: a technique whereby a racemic compound is reacted with an enantiomerically pure reagent (the chiral auxiliary) that converts the individual enantiomers to diastereomers. The resulting diastereomers are then separated by chromatography or crystallization by virtue of their now more distinct structural differences and the chiral auxiliary later removed to obtain the desired enantiomer;

vii) first- and second-order asymmetric transformations: a technique whereby diastereomers from the racemate equilibrate to yield a preponderance in solution of the diastereomer from the desired enantiomer or where preferential crystallization of the diastereomer from the desired enantiomer perturbs the equilibrium such that eventually in principle all the material is converted to the crystalline diastereomer from the desired enantiomer. The desired enantiomer is then released from the diastereomer;

viii) kinetic resolutions: this technique refers to the achievement of partial or complete resolution of a racemate (or of a further resolution of a partially resolved compound) by virtue of unequal reaction rates of the enantiomers with a chiral, non-racemic reagent or catalyst under kinetic conditions;

ix) enantiospecific synthesis from non-racemic precursors: a synthetic technique whereby the desired enantiomer is obtained from non-chiral starting materials and where the stereochemical integrity is not or is only minimally compromised over the course of the synthesis;

x) chiral liquid chromatography: a technique whereby the enantiomers of a racemate are separated in a liquid mobile phase by virtue of their differing interactions with a stationary phase (including but not limited to via chiral HPLC): The stationary phase can be made of chiral material or the mobile phase can contain an additional chiral material to provoke the differing interactions;

xi) chiral gas chromatography: a technique whereby the racemate is volatilized and enantiomers are separated by virtue of their differing interactions in the gaseous mobile phase with a column containing a fixed non-racemic chiral adsorbent phase;

xii) extraction with chiral solvents: a technique whereby the enantiomers are separated by virtue of preferential dissolution of one enantiomer into a particular chiral solvent;

xiii) transport across chiral membranes: a technique whereby a racemate is placed in contact with a thin membrane barrier. The barrier typically separates two miscible fluids, one containing the racemate, and a driving force such as concentration or pressure differential causes preferential transport across the membrane barrier. Separation occurs as a result of the non-racemic chiral nature of the membrane that allows only one enantiomer of the racemate to pass through.

Chiral chromatography, including but not limited to simulated moving bed chromatography, is used in one embodiment. A wide variety of chiral stationary phases are commercially available.

### DEFINITIONS

Listed below are definitions of various terms used to describe this invention. These definitions apply to the terms as they are used throughout this specification and claims, unless otherwise limited in specific instances, either individually or as part of a larger group.

The term “aryl,” as used herein, refers to a mono- or polycyclic carbocyclic ring system including, but not limited to, phenyl, naphthyl, tetrahydronaphthyl, indanyl, idenyl.

The term “heteroaryl,” as used herein, refers to a mono- or polycyclic aromatic radical having one or more ring atom selected from S, O and N; and the remaining ring atoms are carbon, wherein any N or S contained within the ring may be optionally oxidized. Heteroaryl includes, but is not limited to, pyridinyl, pyrazinyl, pyrimidinyl, pyrrolyl, pyrazolyl, imidazolyl, thiazolyl, oxazolyl, isooxazolyl, thiadiazolyl, oxadiazolyl, thiophenyl, furanyl, quinolinyl, isoquinolinyl, benzimidazolyl, benzooxazolyl, quinoxalinyl.

In accordance with the invention, any of the aryls, substituted aryls, heteroaryls and substituted heteroaryls described herein, can be any aromatic group. Aromatic groups can be substituted or unsubstituted.

The terms “C₁-C₈ alkyl,” or “C₁-C₁₂ alkyl,” as used herein, refer to saturated, straight- or branched-chain hydrocarbon radicals containing between one and eight, or one and twelve carbon atoms, respectively. Examples of C₁-C₈ alkyl radicals include, but are not limited to, methyl, ethyl, propyl, isopropyl, n-butyl, tert-butyl, neopentyl, n-hexyl, heptyl and octyl radicals; and examples of C₁-C₁₂ alkyl radicals include, but are not limited to, ethyl, propyl, isopropyl, n-hexyl, octyl, decyl, dodecyl radicals.

The term “C₂-C₈ alkenyl,” as used herein, refer to straight- or branched-chain hydrocarbon radicals containing from two to eight carbon atoms having at least one carbon-carbon double bond by the removal of a single hydrogen atom. Alkenyl groups include, but are not limited to, for example, ethenyl, propenyl, butenyl, 1-methyl-2-buten-1-yl, heptenyl, octenyl, and the like.

The term “C₂-C₈ alkynyl,” as used herein, refer to straight- or branched-chain hydrocarbon radicals containing from two to eight carbon atoms having at least one carbon-carbon triple bond by the removal of a single hydrogen atom. Representative alkynyl groups include, but are not limited to, for example, ethynyl, 1-propynyl, 1-butynyl, heptynyl, octynyl, and the like.

The term “C₃-C₈-cycloalkyl”, or “C₃-C₁₂-cycloalkyl,” as used herein, refers to a monocyclic or polycyclic saturated carbocyclic ring compound. Examples of C₃-C₈-cycloalkyl include, but not limited to, cyclopropyl, cyclobutyl, cyclopentyl, cyclohexyl, cyclopentyl and cyclooctyl; and examples of C₃-C₁₂-cycloalkyl include, but not limited to, cyclopropyl, cyclobutyl, cyclopentyl, cyclohexyl, bicyclo[2.2.1]heptyl, and bicyclo [2.2.2]octyl.

The terms “C₂-C₈ alkylene,” or “C₂-C₈ alkenylene,” as used herein, refer to saturated or unsaturated respectively, straight- or branched-chain hydrocarbon di-radicals containing between two and eight carbon atoms, while the diradical may reside at the same or different carbon atoms.

The term “C₃-C₈ cycloalkenyl”, or “C₃-C₁₂ cycloalkenyl” as used herein, refers to monocyclic or polycyclic carbocyclic ring compound having at least one carbon-carbon double bond. Examples of C₃-C₈ cycloalkenyl include, but not limited to, cyclopropenyl, cyclobutenyl, cyclopentenyl, cyclohexenyl, cycloheptenyl, cyclooctenyl, and the like; and examples of C₃-C₁₂ cycloalkenyl include, but not limited to, cyclopropenyl, cyclobutenyl, cyclopentenyl, cyclohexenyl, cycloheptenyl, cyclooctenyl, and the like.

It is understood that any alkyl, alkenyl, alkynyl and cycloalkyl moiety described herein can also be an aliphatic group, an alicyclic group or a heterocyclic group. An “aliphatic” group is a non-aromatic moiety that may contain any combination of carbon atoms, hydrogen atoms, halogen atoms, oxygen, nitrogen or other atoms, and optionally contain one or more units of unsaturation, e.g., double and/or triple bonds. An aliphatic group may be straight chained, branched or cyclic and preferably contains between about 1 and about 24 carbon atoms, more typically between about 1 and about 12 carbon atoms. In addition to aliphatic hydrocarbon groups, aliphatic groups include, for example, polyalkoxyalkyls, such as polyalkylene glycols, polyamines, and polyimines, for example. Such aliphatic groups may be further substituted.

The term “alicyclic,” as used herein, denotes a monovalent group derived from a monocyclic or bicyclic saturated carbocyclic ring compound by the removal of a single hydrogen atom. Examples include, but not limited to, cyclopropyl, cyclobutyl, cyclopentyl, cyclohexyl, bicyclo[2.2.1]heptyl, and bicyclo[2.2.2]octyl. Such alicyclic groups may be further substituted.

The terms “heterocyclic” or “heterocycloalkyl” can be used interchangeably and referred to a non-aromatic ring or a bi- or tri-cyclic group fused system, where (i) each ring system contains at least one heteroatom independently selected from oxygen, sulfur and nitrogen, (ii) each ring system can be saturated or unsaturated (iii) the nitrogen and sulfur heteroatoms may optionally be oxidized, (iv) the nitrogen heteroatom may optionally be quaternized, (v) any of the above rings may be fused to an aromatic ring, and (vi) the remaining ring atoms are carbon atoms which may be optionally oxo-substituted. Representative heterocyclic groups include, but are not limited to, 1,3-dioxolane, pyrrolidinyl, pyrazolinyl, pyrazolidinyl, imidazolinyl, imidazolidinyl, piperidinyl, piperazinyl, oxazolidinyl, isoxazolidinyl, morpholinyl, thiazolidinyl, isothiazolidinyl, quinoxalinyl, pyridazinonyl, and tetrahydrofuryl. Such heterocyclic groups may be further substituted.

The term “substituted” refers to substitution by independent replacement of one, two, or three or more of the hydrogen atoms thereon with substituents including, but not limited to, —F, —Cl, —Br, —I, —OH, protected hydroxy, —NO₂, —CN, —NH₂, protected amino, oxo, thioxo, —NH—C₁-C₁₂-alkyl, —NH—C₂-C₈-alkenyl, —NH—C₂-C₈-alkynyl, —NH—C₃-C₁₂-cycloalkyl, —NH-aryl, —NH-heteroaryl, —NH-heterocycloalkyl, -dialkylamino, -diarylamino, -diheteroarylamino, —O—C₁-C₁₂-alkyl, —O—C₂-C₈-alkenyl, —O—C₂-C₈-alkynyl, —O—C₃-C₁₂-cycloalkyl, —O-aryl, —O-heteroaryl, —O-heterocycloalkyl, —C(O)—C₁-C₁₂-alkyl, —C(O)—C₂-C₈-alkenyl, —C(O)—C₂-C₈-alkynyl, —C(O)—C₃-C₁₂-cycloalkyl, —C(O)-aryl, —C(O)-heteroaryl, —C(O)-heterocycloalkyl, —CONH₂, —CONH—C₁-C₁₂-alkyl, —CONH—C₂-C₈-alkenyl, —CONH—C₂-C₈-alkynyl, —CONH—C₃-C₁₂-cycloalkyl, —CONH-aryl, —CONH-heteroaryl, —CONH-heterocycloalkyl, —OCO₂—C₁-C₁₂-alkyl, —OCO₂—C₂-C₈-alkenyl, —OCO₂—C₂-C₈-alkynyl, —OCO₂—C₃-C₁₂-cycloalkyl, —OCO₂-aryl, —OCO₂-heteroaryl, —OCO₂-heterocycloalkyl, —OCONH₂, —OCONH—C₁-C₁₂-alkyl, —OCONH—C₂-C₈-alkenyl, —OCONH—C₂-C₈-alkynyl, —OCONH—C₃-C₁₂-cycloalkyl, —OCONH-aryl, —OCONH-heteroaryl, —OCONH-heterocycloalkyl, —NHC(O)—C₁-C₁₂-alkyl, —NHC(O)—C₂-C₈-alkenyl, —NHC(O)—C₂-C₈-alkynyl, —NHC(O)—C₃-C₁₂-cycloalkyl, —NHC(O)-aryl, —NHC(O)-heteroaryl, —NHC(O)-heterocycloalkyl, —NHCO₂—C₁-C₁₂-alkyl, —NHCO₂—C₂-C₈-alkenyl, —NHCO₂—C₂-C₈-alkynyl, —NHCO₂—C₃-C₁₂-cycloalkyl, —NHCO₂-aryl, —NHCO₂-heteroaryl, —NHCO₂— heterocycloalkyl, —NHC(O)NH₂, —NHC(O)NH—C₁-C₁₂-alkyl, —NHC(O)NH—C₂-C₈-alkenyl, —NHC(O)NH—C₂-C₈-alkynyl, —NHC(O)NH—C₃-C₁₂-cycloalkyl, —NHC(O)NH-aryl, —NHC(O)NH-heteroaryl, —NHC(O)NH-heterocycloalkyl, NHC(S)NH₂, —NHC(S)NH—C₁-C₁₂-alkyl, —NHC(S)NH—C₂-C₈-alkenyl, —NHC(S)NH—C₂-C₈-alkynyl, —NHC(S)NH—C₃-C₁₂-cycloalkyl, —NHC(S)NH-aryl, —NHC(S)NH-heteroaryl, —NHC(S)NH-heterocycloalkyl, —NHC(NH)NH₂, —NHC(NH)NH—C₁-C₁₂-alkyl, —NHC(NH)NH—C₂-C₈-alkenyl, —NHC(NH)NH—C₂-C₈-alkynyl, —NHC(NH)NH—C₃-C₁₂-cycloalkyl, —NHC(NH)NH-aryl, —NHC(NH)NH-heteroaryl, —NHC(NH)NH-heterocycloalkyl, —NHC(NH)—C₁-C₁₂-alkyl, —NHC(NH)—C₂-C₈-alkenyl, —NHC(NH)—C₂-C₈-alkynyl, —NHC(NH)—C₃-C₁₂-cycloalkyl, —NHC(NH)-aryl, —NHC(NH)-heteroaryl, —NHC(NH)-heterocycloalkyl, —C(NH)NH—C₁-C₁₂-alkyl, —C(NH)NH—C₂-C₈-alkenyl, —C(NH)NH—C₂-C₈-alkynyl, —C(NH)NH—C₃-C₁₂-cycloalkyl, —C(NH)NH-aryl, —C(NH)NH-heteroaryl, —C(NH)NH-heterocycloalkyl, —S(O)—C₁-C₁₂-alkyl, —S(O)—C₂-C₈-alkenyl, —S(O)—C₂-C₈-alkynyl, —S(O)—C₃-C₁₂-cycloalkyl, —S(O)-aryl, —S(O)-heteroaryl, —S(O)-heterocycloalkyl —SO₂NH₂, —SO₂NH—C₁-C₁₂-alkyl, —SO₂NH—C₂-C₈-alkenyl, —SO₂NH—C₂-C₈-alkynyl, —SO₂NH—C₃-C₁₂-cycloalkyl, —SO₂NH-aryl, —SO₂NH-heteroaryl, —SO₂NH-heterocycloalkyl, —NHSO₂—C₁-C₁₂-alkyl, —NHSO₂—C₂-C₈-alkenyl, —NHSO₂—C₂-C₈-alkynyl, —NHSO₂—C₃-C₁₂-cycloalkyl, —NHSO₂-aryl, —NHSO₂-heteroaryl, —NHSO₂-heterocycloalkyl, —CH₂NH₂, —CH₂SO₂CH₃, -aryl, -arylalkyl, -heteroaryl, -heteroarylalkyl, -heterocycloalkyl, —C₃-C₁₂-cycloalkyl, polyalkoxyalkyl, polyalkoxy, -methoxymethoxy, -methoxyethoxy, —SH, —S—C₁-C₁₂-alkyl, —S—C₂-C₈-alkenyl, —S—C₂-C₈-alkynyl, —S—C₃-C₁₂-cycloalkyl, —S-aryl, —S-heteroaryl, —S-heterocycloalkyl, or methylthiomethyl. It is understood that the aryls, heteroaryls, alkyls, and the like can be further substituted.

The term “halogen,” as used herein, refers to an atom selected from fluorine, chlorine, bromine and iodine.

The term “hydroxy activating group”, as used herein, refers to a labile chemical moiety which is known in the art to activate a hydroxyl group so that it will depart during synthetic procedures such as in a substitution or an elimination reaction. Examples of hydroxyl activating group include, but not limited to, mesylate, tosylate, triflate, p-nitrobenzoate, phosphonate and the like.

The term “activated hydroxy”, as used herein, refers to a hydroxy group activated with a hydroxyl activating group, as defined above, including mesylate, tosylate, triflate, p-nitrobenzoate, phosphonate groups, for example.

The term “hydroxy protecting group,” as used herein, refers to a labile chemical moiety which is known in the art to protect a hydroxyl group against undesired reactions during synthetic procedures. After said synthetic procedure(s) the hydroxy protecting group as described herein may be selectively removed. Hydroxy protecting groups as known in the art are described generally in T. H. Greene and P. G. M. Wuts, Protective Groups in Organic Synthesis, 3rd edition, John Wiley & Sons, New York (1999). Examples of hydroxyl protecting groups include benzyloxycarbonyl, 4-nitrobenzyloxycarbonyl, 4-bromobenzyloxycarbonyl, 4-methoxybenzyloxycarbonyl, methoxycarbonyl, tert-butoxycarbonyl, isopropoxycarbonyl, diphenylmethoxycarbonyl, 2,2,2-trichloroethoxycarbonyl, 2-(trimethylsilyl)ethoxycarbonyl, 2-furfuryloxycarbonyl, allyloxycarbonyl, acetyl, formyl, chloroacetyl, trifluoroacetyl, methoxyacetyl, phenoxyacetyl, benzoyl, methyl, t-butyl, 2,2,2-trichloroethyl, 2-trimethylsilyl ethyl, 1,1-dimethyl-2-propenyl, 3-methyl-3-butenyl, allyl, benzyl, para-methoxybenzyldiphenylmethyl, triphenylmethyl(trityl), tetrahydrofuryl, methoxymethyl, methylthiomethyl, benzyloxymethyl, 2,2,2-triehloroethoxymethyl, 2-(trimethylsilyl)ethoxymethyl, methanesulfonyl, para-toluenesulfonyl, trimethylsilyl, triethylsilyl, triisopropylsilyl, and the like. Preferred hydroxyl protecting groups for the present invention are acetyl (Ac or —C(O)CH₃), benzoyl (Bz or —C(O)C₆H₅), and trimethylsilyl (TMS or —Si(CH₃)₃).

The term “protected hydroxy,” as used herein, refers to a hydroxy group protected with a hydroxy protecting group, as defined above, including benzoyl, acetyl, trimethylsilyl, triethylsilyl, methoxymethyl groups, for example.

The term “hydroxy prodrug group”, as used herein, refers to a promoiety group which is known in the art to change the physicochemical, and hence the biological properties of a parent drug in a transient manner by covering or masking the hydroxy group. After said synthetic procedure(s), the hydroxy prodrug group as described herein must be capable of reverting back to hydroxy group in vivo. Hydroxy prodrug groups as known in the art are described generally in Kenneth B. Sloan, Prodrugs, Topical and Ocular Drug Delivery, (Drugs and the Pharmaceutical Sciences; Volume 53), Marcel Dekker, Inc., New York (1992).

The term “amino protecting group,” as used herein, refers to a labile chemical moiety which is known in the art to protect an amino group against undesired reactions during synthetic procedures. After said synthetic procedure(s) the amino protecting group as described herein may be selectively removed. Amino protecting groups as known in the art are described generally in T. H. Greene and P. G. M. Wuts, Protective Groups in Organic Synthesis, 3rd edition, John Wiley & Sons, New York (1999). Examples of amino protecting groups include, but are not limited to, t-butoxycarbonyl, 9-fluorenylmethoxycarbonyl, benzyloxycarbonyl, and the like.

The term “leaving group” means a functional group or atom which can be displaced by another functional group or atom in a substitution reaction, such as a nucleophilic substitution reaction. By way of example, representative leaving groups include chloro, bromo and iodo groups; sulfonic ester groups, such as mesylate, tosylate, brosylate, nosylate and the like; and acyloxy groups, such as acetoxy, trifluoroacetoxy and the like.

The term “protected amino,” as used herein, refers to an amino group protected with an amino protecting group as defined above.

The term “aprotic solvent,” as used herein, refers to a solvent that is relatively inert to proton activity, i.e., not acting as a proton-donor. Examples include, but are not limited to, hydrocarbons, such as hexane and toluene, for example, halogenated hydrocarbons, such as, for example, methylene chloride, ethylene chloride, chloroform, and the like, heterocyclic compounds, such as, for example, tetrahydrofuran and N-methylpyrrolidinone, and ethers such as diethyl ether, bis-methoxymethyl ether. Such compounds are well known to those skilled in the art, and it will be obvious to those skilled in the art that individual solvents or mixtures thereof may be preferred for specific compounds and reaction conditions, depending upon such factors as the solubility of reagents, reactivity of reagents and preferred temperature ranges, for example. Further discussions of aprotic solvents may be found in organic chemistry textbooks or in specialized monographs, for example: Organic Solvents Physical Properties and Methods of Purification, 4th ed., edited by John A. Riddick et al, Vol. II, in the Techniques of Chemistry Series, John Wiley & Sons, NY, 1986.

The term “protic solvent’ as used herein, refers to a solvent that tends to provide protons, such as an alcohol, for example, methanol, ethanol, propanol, isopropanol, butanol, t-butanol, and the like. Such solvents are well known to those skilled in the art, and it will be obvious to those skilled in the art that individual solvents or mixtures thereof may be preferred for specific compounds and reaction conditions, depending upon such factors as the solubility of reagents, reactivity of reagents and preferred temperature ranges, for example. Further discussions of protogenic solvents may be found in organic chemistry textbooks or in specialized monographs, for example: Organic Solvents Physical Properties and Methods of Purification, 4th ed., edited by John A. Riddick et al., Vol. II, in the Techniques of Chemistry Series, John Wiley & Sons, NY, 1986.

Combinations of substituents and variables envisioned by this invention are only those that result in the formation of stable compounds. The term “stable”, as used herein, refers to compounds which possess stability sufficient to allow manufacture and which maintains the integrity of the compound for a sufficient period of time to be useful for the purposes detailed herein (e.g., therapeutic or prophylactic administration to a subject).

The synthesized compounds can be separated from a reaction mixture and further purified by a method such as column chromatography, high pressure liquid chromatography, or recrystallization. As can be appreciated by the skilled artisan, further methods of synthesizing the compounds of the Formula herein will be evident to those of ordinary skill in the art. Additionally, the various synthetic steps may be performed in an alternate sequence or order to give the desired compounds. Synthetic chemistry transformations and protecting group methodologies (protection and deprotection) useful in synthesizing the compounds described herein are known in the art and include, for example, those such as described in R. Larock, Comprehensive Organic Transformations, 2^{nd} Ed. Wiley-VCH (1999); T. W. Greene and P. G. M. Wuts, Protective Groups in Organic Synthesis, 3rd Ed., John Wiley and Sons (1999); L. Fieser and M. Fieser, Fieser and Fieser's Reagents for Organic Synthesis, John Wiley and Sons (1994); and L. Paquette, ed., Encyclopedia of Reagents for Organic Synthesis, John Wiley and Sons (1995), and subsequent editions thereof.

The term “subject” as used herein refers to an animal. Preferably the animal is a mammal. More preferably the mammal is a human. A subject also refers to, for example, dogs, cats, horses, cows, pigs, guinea pigs, fish, birds and the like.

The compounds of this invention may be modified by appending appropriate functionalities to enhance selective biological properties. Such modifications are known in the art and may include those which increase biological penetration into a given biological system (e.g., blood, lymphatic system, central nervous system), increase oral availability, increase solubility to allow administration by injection, alter metabolism and alter rate of excretion.

The compounds described herein contain one or more asymmetric centers and thus give rise to enantiomers, diastereomers, and other stereoisomeric forms that may be defined, in terms of absolute stereochemistry, as (R)- or (S)-, or as (D)- or (L)- for amino acids. The present invention is meant to include all such possible isomers, as well as their racemic and optically pure forms. Optical isomers may be prepared from their respective optically active precursors by the procedures described above, or by resolving the racemic mixtures. The resolution can be carried out in the presence of a resolving agent, by chromatography or by repeated crystallization or by some combination of these techniques which are known to those skilled in the art. Further details regarding resolutions can be found in Jacques, et al., Enantiomers, Racemates, and Resolutions (John Wiley & Sons, 1981). When the compounds described herein contain olefinic double bonds, other unsaturation, or other centers of geometric asymmetry, and unless specified otherwise, it is intended that the compounds include both E and Z geometric isomers or cis- and trans-isomers. Likewise, all tautomeric forms are also intended to be included. Tautomers may be in cyclic or acyclic. The configuration of any carbon-carbon double bond appearing herein is selected for convenience only and is not intended to designate a particular configuration unless the text so states; thus a carbon-carbon double bond or carbon-heteroatom double bond depicted arbitrarily herein as trans may be cis, trans, or a mixture of the two in any proportion.

As used herein, the term “pharmaceutically acceptable salt” refers to those salts which are, within the scope of sound medical judgment, suitable for use in contact with the tissues of humans and lower animals without undue toxicity, irritation, allergic response and the like, and are commensurate with a reasonable benefit/risk ratio. Pharmaceutically acceptable salts are well known in the art. For example, S. M. Berge, et al. describes pharmaceutically acceptable salts in detail in J. Pharmaceutical Sciences, 66: 1-19 (1977). The salts can be prepared in situ during the final isolation and purification of the compounds of the invention, or separately by reacting the free base function with a suitable organic acid. Examples of pharmaceutically acceptable salts include, but are not limited to, nontoxic acid addition salts are salts of an amino group formed with inorganic acids such as hydrochloric acid, hydrobromic acid, phosphoric acid, sulfuric acid and perchloric acid or with organic acids such as acetic acid, maleic acid, tartaric acid, citric acid, succinic acid or malonic acid or by using other methods used in the art such as ion exchange. Other pharmaceutically acceptable salts include, but are not limited to, adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, p-toluenesulfonate, undecanoate, valerate salts, and the like. Representative alkali or alkaline earth metal salts include sodium, lithium, potassium, calcium, magnesium, and the like. Further pharmaceutically acceptable salts include, when appropriate, nontoxic ammonium, quaternary ammonium, and amine cations formed using counterions such as halide, hydroxide, carboxylate, sulfate, phosphate, nitrate, alkyl having from 1 to 6 carbon atoms, sulfonate and aryl sulfonate.

As used herein, the term “pharmaceutically acceptable ester” refers to esters which hydrolyze in vivo and include those that break down readily in the human body to leave the parent compound or a salt thereof. Suitable ester groups include, for example, those derived from pharmaceutically acceptable aliphatic carboxylic acids, particularly alkanoic, alkenoic, cycloalkanoic and alkanedioic acids, in which each alkyl or alkenyl moiety advantageously has not more than 6 carbon atoms. Examples of particular esters include, but are not limited to, formates, acetates, propionates, butyrates, acrylates and ethylsuccinates.

The term “pharmaceutically acceptable prodrugs” as used herein refers to those prodrugs of the compounds of the present invention which are, within the scope of sound medical judgment, suitable for use in contact with the tissues of humans and lower animals with undue toxicity, irritation, allergic response, and the like, commensurate with a reasonable benefit/risk ratio, and effective for their intended use, as well as the zwitterionic forms, where possible, of the compounds of the present invention. “Prodrug”, as used herein means a compound which is convertible in vivo by metabolic means (e.g. by hydrolysis) to a compound of the invention. Various forms of prodrugs are known in the art, for example, as discussed in Bundgaard, (ed.), Design of Prodrugs, Elsevier (1985); Widder, et al. (ed.), Methods in Enzymology, vol. 4, Academic Press (1985); Krogsgaard-Larsen, et al., (ed). “Design and Application of Prodrugs, Textbook of Drug Design and Development, Chapter 5, 113-191 (1991); Bundgaard, et al., Journal of Drug Deliver Reviews, 8:1-38 (1992); Bundgaard, J. of Pharmaceutical Sciences, 77:285 et seq. (1988); Higuchi and Stella (eds.) Prodrugs as Novel Drug Delivery Systems, American Chemical Society (1975); Bernard Testa & Joachim Mayer, “Hydrolysis In Drug And Prodrug Metabolism: Chemistry, Biochemistry And Enzymology,” John Wiley and Sons, Ltd. (2002); and J. Rautio et al, “Prodrugs: design and clinical applications”, Nature Review-Drug Discovery, 7, 255-270 (2008).

The present invention also relates to solvates of the compounds of Formula (I), for example hydrates.

This invention also encompasses pharmaceutical compositions containing, and methods of treating viral infections through administering, pharmaceutically acceptable prodrugs of compounds of the invention. For example, compounds of the invention having free amino, amido, hydroxy or carboxylic groups can be converted into prodrugs. Prodrugs include compounds wherein an amino acid residue, or a polypeptide chain of two or more (e.g., two, three or four) amino acid residues is covalently joined through an amide or ester bond to a free amino, hydroxy or carboxylic acid group of compounds of the invention. The amino acid residues include but are not limited to the 20 naturally occurring amino acids commonly designated by three letter symbols and also includes 4-hydroxyproline, hydroxylysine, demosine, isodemosine, 3-methylhistidine, norvalin, beta-alanine, gamma-aminobutyric acid, citrulline, homocysteine, homoserine, ornithine and methionine sulfone. Additional types of prodrugs are also encompassed. For instance, free carboxyl groups can be derivatized as amides or alkyl esters. Free hydroxy groups may be derivatized using groups including but not limited to hemisuccinates, phosphate esters, dimethylaminoacetates, and phosphoryloxymethyloxycarbonyls, as outlined in Advanced Drug Delivery Reviews, 1996, 19, 115. Carbamate prodrugs of hydroxy and amino groups are also included, as are carbonate prodrugs, sulfonate esters and sulfate esters of hydroxy groups. Derivatization of hydroxy groups as (acyloxy)methyl and (acyloxy)ethyl ethers wherein the acyl group may be an alkyl ester, optionally substituted with groups including but not limited to ether, amine and carboxylic acid functionalities, or where the acyl group is an amino acid ester as described above, are also encompassed. Prodrugs of this type are described in J. Med. Chem. 1996, 39, 10. Free amines can also be derivatized as amides, sulfonamides or phosphonamides. All of these prodrug moieties may incorporate groups including but not limited to ether, amine and carboxylic acid functionalities.

### Pharmaceutical Compositions

The pharmaceutical compositions of the present invention comprise a therapeutically effective amount of a compound of the present invention formulated together with one or more pharmaceutically acceptable carriers or excipients.

As used herein, the term “pharmaceutically acceptable carrier or excipient” means a non-toxic, inert solid, semi-solid or liquid filler, diluent, encapsulating material or formulation auxiliary of any type. Some examples of materials which can serve as pharmaceutically acceptable carriers are sugars such as lactose, glucose and sucrose; starches such as corn starch and potato starch; cellulose and its derivatives such as sodium carboxymethyl cellulose, ethyl cellulose and cellulose acetate; powdered tragacanth; malt; gelatin; talc; excipients such as cocoa butter and suppository waxes; oils such as peanut oil, cottonseed oil, safflower oil, sesame oil, olive oil, corn oil and soybean oil; glycols such as propylene glycol; esters such as ethyl oleate and ethyl laurate; agar; buffering agents such as magnesium hydroxide and aluminum hydroxide; alginic acid; pyrogen-free water; isotonic saline; Ringer's solution; ethyl alcohol, and phosphate buffer solutions, as well as other non-toxic compatible lubricants such as sodium lauryl sulfate and magnesium stearate, as well as coloring agents, releasing agents, coating agents, sweetening, flavoring and perfuming agents, preservatives and antioxidants can also be present in the composition, according to the judgment of the formulator.

The pharmaceutical compositions of this invention may be administered orally, parenterally, by inhalation spray, topically, rectally, nasally, buccally, vaginally or via an implanted reservoir, preferably by oral administration or administration by injection. The pharmaceutical compositions of this invention may contain any conventional non-toxic pharmaceutically-acceptable carriers, adjuvants or vehicles. In some cases, the pH of the formulation may be adjusted with pharmaceutically acceptable acids, bases or buffers to enhance the stability of the formulated compound or its delivery form. The term parenteral as used herein includes subcutaneous, intracutaneous, intravenous, intramuscular, intraarticular, intraarterial, intrasynovial, intrasternal, intrathecal, intralesional and intracranial injection or infusion techniques.

Liquid dosage forms for oral administration include pharmaceutically acceptable emulsions, microemulsions, solutions, suspensions, syrups and elixirs. In addition to the active compounds, the liquid dosage forms may contain inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof. Besides inert diluents, the oral compositions can also include adjuvants such as wetting agents, emulsifying and suspending agents, sweetening, flavoring, and perfuming agents.

Injectable preparations, for example, sterile injectable aqueous or oleaginous suspensions, may be formulated according to the known art using suitable dispersing or wetting agents and suspending agents. The sterile injectable preparation may also be a sterile injectable solution, suspension or emulsion in a nontoxic parenterally acceptable diluent or solvent, for example, as a solution in 1,3-butanediol. Among the acceptable vehicles and solvents that may be employed are water, Ringer's solution, U.S.P. and isotonic sodium chloride solution. In addition, sterile, fixed oils are conventionally employed as a solvent or suspending medium. For this purpose any bland fixed oil can be employed including synthetic mono- or diglycerides. In addition, fatty acids such as oleic acid are used in the preparation of injectables.

The injectable formulations can be sterilized, for example, by filtration through a bacterial-retaining filter, or by incorporating sterilizing agents in the form of sterile solid compositions which can be dissolved or dispersed in sterile water or other sterile injectable medium prior to use.

In order to prolong the effect of a drug, it is often desirable to slow the absorption of the drug from subcutaneous or intramuscular injection. This may be accomplished by the use of a liquid suspension of crystalline or amorphous material with poor water solubility. The rate of absorption of the drug then depends upon its rate of dissolution, which, in turn, may depend upon crystal size and crystalline form. Alternatively, delayed absorption of a parenterally administered drug form is accomplished by dissolving or suspending the drug in an oil vehicle. Injectable depot forms are made by forming microencapsule matrices of the drug in biodegradable polymers such as polylactide-polyglycolide. Depending upon the ratio of drug to polymer and the nature of the particular polymer employed, the rate of drug release can be controlled. Examples of other biodegradable polymers include poly(orthoesters) and poly(anhydrides). Depot injectable formulations are also prepared by entrapping the drug in liposomes or microemulsions that are compatible with body tissues.

Compositions for rectal or vaginal administration are preferably suppositories which can be prepared by mixing the compounds of this invention with suitable non-irritating excipients or carriers such as cocoa butter, polyethylene glycol or a suppository wax which are solid at ambient temperature but liquid at body temperature and therefore melt in the rectum or vaginal cavity and release the active compound.

Solid dosage forms for oral administration include capsules, tablets, pills, powders, and granules. In such solid dosage forms, the active compound is mixed with at least one inert, pharmaceutically acceptable excipient or carrier such as sodium citrate or dicalcium phosphate and/or: a) fillers or extenders such as starches, lactose, sucrose, glucose, mannitol, and silicic acid, b) binders such as, for example, carboxymethylcellulose, alginates, gelatin, polyvinylpyrrolidinone, sucrose, and acacia, c) humectants such as glycerol, d) disintegrating agents such as agar-agar, calcium carbonate, potato or tapioca starch, alginic acid, certain silicates, and sodium carbonate, e) solution retarding agents such as paraffin, f) absorption accelerators such as quaternary ammonium compounds, g) wetting agents such as, for example, cetyl alcohol and glycerol monostearate, h) absorbents such as kaolin and bentonite clay, and i) lubricants such as talc, calcium stearate, magnesium stearate, solid polyethylene glycols, sodium lauryl sulfate, and mixtures thereof. In the case of capsules, tablets and pills, the dosage form may also comprise buffering agents.

Solid compositions of a similar type may also be employed as fillers in soft and hard-filled gelatin capsules using such excipients as lactose or milk sugar as well as high molecular weight polyethylene glycols and the like.

The solid dosage forms of tablets, dragees, capsules, pills, and granules can be prepared with coatings and shells such as enteric coatings and other coatings well known in the pharmaceutical formulating art. They may optionally contain opacifying agents and can also be of a composition that they release the active ingredient(s) only, or preferentially, in a certain part of the intestinal tract, optionally, in a delayed manner. Examples of embedding compositions that can be used include polymeric substances and waxes.

Dosage forms for topical or transdermal administration of a compound of this invention include ointments, pastes, creams, lotions, gels, powders, solutions, sprays, inhalants or patches. The active component is admixed under sterile conditions with a pharmaceutically acceptable carrier and any needed preservatives or buffers as may be required. Ophthalmic formulation, ear drops, eye ointments, powders and solutions are also contemplated as being within the scope of this invention.

The ointments, pastes, creams and gels may contain, in addition to an active compound of this invention, excipients such as animal and vegetable fats, oils, waxes, paraffins, starch, tragacanth, cellulose derivatives, polyethylene glycols, silicones, bentonites, silicic acid, talc and zinc oxide, or mixtures thereof.

Powders and sprays can contain, in addition to the compounds of this invention, excipients such as lactose, talc, silicic acid, aluminum hydroxide, calcium silicates and polyamide powder, or mixtures of these substances. Sprays can additionally contain customary propellants such as chlorofluorohydrocarbons.

Transdermal patches have the added advantage of providing controlled delivery of a compound to the body. Such dosage forms can be made by dissolving or dispensing the compound in the proper medium. Absorption enhancers can also be used to increase the flux of the compound across the skin. The rate can be controlled by either providing a rate controlling membrane or by dispersing the compound in a polymer matrix or gel.

For pulmonary delivery, a therapeutic composition of the invention is formulated and administered to the patient in solid or liquid particulate form by direct administration e.g., inhalation into the respiratory system. Solid or liquid particulate forms of the active compound prepared for practicing the present invention include particles of respirable size: that is, particles of a size sufficiently small to pass through the mouth and larynx upon inhalation and into the bronchi and alveoli of the lungs. Delivery of aerosolized therapeutics, particularly aerosolized antibiotics, is known in the art (see, for example U.S. Pat. No. 5,767,068 to VanDevanter et al., U.S. Pat. No. 5,508,269 to Smith et al, and WO 98/43,650 by Montgomery, all of which are incorporated herein by reference). A discussion of pulmonary delivery of antibiotics is also found in U.S. Pat. No. 6,014,969, incorporated herein by reference.

According to the methods of treatment of the present invention, viral infections, conditions are treated or prevented in a patient such as a human or another animal by administering to the patient a therapeutically effective amount of a compound of the invention, in such amounts and for such time as is necessary to achieve the desired result.

By a “therapeutically effective amount” of a compound of the invention is meant an amount of the compound which confers a therapeutic effect on the treated subject, at a reasonable benefit/risk ratio applicable to any medical treatment. The therapeutic effect may be objective (i.e., measurable by some test or marker) or subjective (i.e., subject gives an indication of or feels an effect). A therapeutically effective amount of the compound described above may range from about 0.1 mg/Kg to about 500 mg/Kg, preferably from about 1 to about 50 mg/Kg. Effective doses will also vary depending on route of administration, as well as the possibility of co-usage with other agents. It will be understood, however, that the total daily usage of the compounds and compositions of the present invention will be decided by the attending physician within the scope of sound medical judgment. The specific therapeutically effective dose level for any particular patient will depend upon a variety of factors including the disorder being treated and the severity of the disorder; the activity of the specific compound employed; the specific composition employed; the age, body weight, general health, sex and diet of the patient; the time of administration, route of administration, and rate of excretion of the specific compound employed; the duration of the treatment; drugs used in combination or contemporaneously with the specific compound employed; and like factors well known in the medical arts.

The total daily dose of the compounds of this invention administered to a human or other animal in single or in divided doses can be in amounts, for example, from 0.01 to 50 mg/kg body weight or more usually from 0.1 to 25 mg/kg body weight. Single dose compositions may contain such amounts or submultiples thereof to make up the daily dose. In general, treatment regimens according to the present invention comprise administration to a patient in need of such treatment from about 10 mg to about 1000 mg of the compound(s) of this invention per day in single or multiple doses.

The compounds of the invention described herein can, for example, be administered by injection, intravenously, intraarterially, subdermally, intraperitoneally, intramuscularly, or subcutaneously; or orally, buccally, nasally, transmucosally, topically, in an ophthalmic preparation, or by inhalation, with a dosage ranging from about 0.1 to about 500 mg/kg of body weight, alternatively dosages between 1 mg and 1000 mg/dose, every 4 to 120 hours, or according to the requirements of the particular drug. The methods herein contemplate administration of a therapeutically effective amount of compound or compound composition to achieve the desired or stated effect. Typically, the pharmaceutical compositions of this invention will be administered from about 1 to about 6 times per day or alternatively, as a continuous infusion. Such administration can be used as a chronic or acute therapy. The amount of active ingredient that may be combined with pharmaceutically exipients or carriers to produce a single dosage form will vary depending upon the host treated and the particular mode of administration. A typical preparation will contain from about 5% to about 95% active compound (w/w). Alternatively, such preparations may contain from about 20% to about 80% active compound.

Lower or higher doses than those recited above may be required. Specific dosage and treatment regimens for any particular patient will depend upon a variety of factors, including the activity of the specific compound employed, the age, body weight, general health status, sex, diet, time of administration, rate of excretion, drug combination, the severity and course of the disease, condition or symptoms, the patient's disposition to the disease, condition or symptoms, and the judgment of the treating physician.

Upon improvement of a patient's condition, a maintenance dose of a compound, composition or combination of this invention may be administered, if necessary. Subsequently, the dosage or frequency of administration, or both, may be reduced, as a function of the symptoms, to a level at which the improved condition is retained when the symptoms have been alleviated to the desired level. Patients may, however, require intermittent treatment on a long-term basis upon any recurrence of disease symptoms.

When the compositions of this invention comprise a combination of a compound of the invention described herein and one or more additional therapeutic or prophylactic agents, both the compound and the additional agent should be present at dosage levels of between about 1 to 100%, and more preferably between about 5 to 95% of the dosage normally administered in a monotherapy regimen. The additional agents may be administered separately, as part of a multiple dose regimen, from the compounds of this invention. Alternatively, those agents may be part of a single dosage form, mixed together with the compounds of this invention in a single composition.

The said “additional therapeutic or prophylactic agents” includes but not limited to, immune therapies (eg. interferon), therapeutic vaccines, antifibrotic agents, anti-inflammatory agents such as corticosteroids or NSAIDs, bronchodilators such as beta-2 adrenergic agonists and xanthines (e.g. theophylline), mucolytic agents, anti-muscarinics, anti-leukotrienes, inhibitors of cell adhesion (e.g. ICAM antagonists), anti-oxidants (eg N-acetylcysteine), cytokine agonists, cytokine antagonists, lung surfactants and/or antimicrobial and anti-viral agents (eg ribavirin and amantidine). The compositions according to the invention may also be used in combination with gene replacement therapy.

Unless otherwise defined, all technical and scientific terms used herein are accorded the meaning commonly known to one of ordinary skill in the art. All publications, patents, published patent applications, and other references mentioned herein are hereby incorporated by reference in their entirety.

### Pharmaceutically Acceptable Derivatives

The compound of the present invention can be administered as any derivative that upon administration to the recipient, is capable of providing directly or indirectly, the parent compound. Further, the modifications can affect the biological activity of the compound, in some cases increasing the activity over the parent compound. This can easily be assessed by preparing the derivative and testing its antiviral and anti-proliferative activity according to the methods described herein, or other method known to those skilled in the art.

In cases where compounds are sufficiently basic or acidic to form stable nontoxic acid or base salts, administration of the compound as a pharmaceutically acceptable salt may be appropriate. Examples of pharmaceutically acceptable salts are organic acid addition salts formed with acids, which form a physiological acceptable anion, for example, tosylate, methanesulfonate, acetate, citrate, malonate, tartarate, succinate, benzoate, ascorbate, α-ketoglutarate and α-glycerophosphate. Suitable inorganic salts may also be formed, including sulfate, nitrate, bicarbonate, and carbonate salts.

Pharmaceutically acceptable salts may be obtained using standard procedures well known in the art, for example by reacting a sufficiently basic compound such as an amine with a suitable acid affording a physiologically acceptable anion. Alkali metal (for example, sodium, potassium or lithium) or alkaline earth metal (for example calcium) salts of carboxylic acids can also be made.

Any of the nucleosides described herein can be administrated as a nucleotide prodrug to increase the activity, bioavailability, stability or otherwise alter the properties of the nucleoside. A number of nucleotide prodrug ligands are known. In general, alkylation, acylation or other lipophilic modification of the mono, di or triphosphate of the nucleoside will increase the stability of the nucleotide. Examples of substituent groups that can replace one or more hydrogens on the phosphate moiety are alkyl, aryl, steroids, carbohydrates, including sugars, 1,2-diacylglycerol and alcohols. Many are described in R. Jones and N. Bischofberger, Antiviral Research, 27 (1995) 1-17. Any of these can be used in combination with the disclosed nucleosides to achieve a desired effect.

The active nucleoside can also be provided as a 5′-phosphoether lipid or a 5′-ether lipid, as disclosed in the following references, which are incorporated by reference herein: Kucera, L. S. et al 1990. “Novel membrane interactive ether lipid analogs that inhibit infectious HIV-1 production and induce defective virus formation.” AIDS Res. Hum. Retro Viruses. 6:491-501; Piantadosi, C., J. et al 1991. “Synthesis and evaluation of novel ether lipid nucleoside conjugates for anti-HIV activity.” J. Med. Chem. 34:1408.1414; Hosteller, K. Y. et al 1992. “Greatly enhanced inhibition of human immunodeficiency virus type 1 replication in CEM and HT4-6C cells by 3′-deoxythymidine diphosphate dimyristoylglycerol, a lipid prodrug of 3′-deoxythymidine.” Antimicrob. Agents Chemother. 36:2025.2029; Hosetler, K. Y., et al 1990. “Synthesis and antiretroviral activity of phospholipid analogs of azidothymidine and other antiviral nucleosides.” J. Biol. Chem. 265:61127.

Nonlimiting examples of U.S. patents that disclose suitable lipophilic substituents that can be covalently incorporated into the nucleoside, preferably at the 5′-OH position of the nucleoside or lipophilic preparations, include U.S. Pat. Nos. 5,149,794 (Sep. 22, 1992, Yatvin et al.); 5,194,654 (Mar. 16, 1993, Hostetler et al., 5,223,263 (Jun. 29, 1993, Hostetler et al.); 5,256,641 (Oct. 26, 1993, Yatvin et al.); 5,411,947 (May 2, 1995, Hostetler et al.); 5,463,092 (Oct. 31, 1995, Hostetler et al.); 5,543,389 (Aug. 6, 1996, Yatvin et al.); 5,543,390 (Aug. 6, 1996, Yatvin et al.); 5,543,391 (Aug. 6, 1996, Yatvin et al.); and 5,554,728 (Sep. 10, 1996; Basava et al.), all of which are incorporated herein by reference. Foreign patent publications that disclose lipophilic substituents that can be attached to the nucleosides of the present invention, or lipophilic preparations, include WO 89/02733, WO 90/00555, WO 91/16920, WO 91/18914, WO 93/00910, WO 94/26273, WO 96/15132, EP 0 350 287, and WO 91/19721.

Nonlimiting examples of nucleotide prodrugs are described in the following references: Ho, D. H. W. (1973) “Distribution of Kinase and deaminase of 1β-D-arabinofuranosylcytosine in tissues of man and muse.” Cancer Res. 33, 2816-2820; Holy, A. (1993) Isopolar phosphorous-modified nucleotide analogues,” In: De Clercq (Ed.), Advances in Antiviral Drug Design, Vol. 1, JAI Press, pp. 179-231; Hong, C. I., et al (1979a) “Synthesis and antitumor activity of 1-β-D-arabino-furanosylcytosine conjugates of cortisol and cortisone.” Bicohem. Biophys. Rs. Commun. 88, 1223-1229; Hong, C. I., et al (1980) “Nucleoside conjugates as potential antitumor agents. 3. Synthesis and antitumor activity of 1-(β-D-arabinofuranosyl)cytosine conjugates of corticosteriods and selected lipophilic alcohols.” J. Med. Chem. 28, 171-177; Hosteller, K. Y., et al (1991); “Phosphatidylazidothymidine: mechanism of antiretroviral action in CEM cells.” J. Biol. Chem. 266, 11714-11717; Hosteller, K. Y., et al (1994a) “Antiviral activity of phosphatidyl-dideoxycytidine in hepatitis B-infected cells and enhanced hepatic uptake in mice.” Antiviral Res. 24, 59-67; Hosteller, K. Y., et al (1994b) “Phosphatidylazidothymidine and phosphatidyl-ddC: Assessment of uptake in mouse lymphoid tissues and antiviral activities in human immunodeficiency virus-infected cells and in rauscher leukemia virus-infected mice.” Antimicrobial Agents Chemother. 38, 2792-2797; Hunston, R. N., et al (1984) “Synthesis and biological properties of some cyclic phosphotriesters derived from 2′-deoxy-5-fluorouridine.” J. Med. Chem. 27, 440-444; Ji, Y. H., et al (1990); “Monophosphoric acid esters of 7-R-hydroxycholesterol and of pyrimidine nucleoside as potential antitumor agents: synthesis and preliminary evaluation of antitumor activity.” J. Med. Chem. 33 2264-2270; Jones, A. S., et al (1984) “Synthesis, properties, and biological activity of some nucleoside cyclic phosphoramidates.” J. Chem. Soc. Perkin Trans. 1, 1471-1474; Juodka, B. A. and Smrt, J. (1974) “Synthesis of diribonucleoside phosph (P→N) amino acid derivatives.” Coll. Czech. Chem. Comm. 39, 363-968; Kataoka, S., et al (1989) “Alkylated cAMP derivatives; selective synthesis and biological activities.” Nucleic Acids Res. Sym. Ser. 21, 1-2; Kataoka, S., et al “(cAMP) benzyl and methyl triesters. “Heterocycles 32, 1351-1356; Kinchington, D., et al (1992) “Comparison of antiviral effects of zidovudine phosphoramidate and phosphorodiamidate derivates against HIV and ULV in vitro.” Antiviral Chem. Chemother. 3, 107-112; Kodama, K., et al (1989) “Antitumor activity and pharmacology of 1-β-D-arabinofuranosylcytosine-5′-stearylphosphate; an orally active derivative of 1-β-D-arabinofuranosylcytosine.” Jpn. J. Cancer Res. 80, 679-685; Korty, M. and Engels, J. (1979) “The effects of adenosine- and guanosine 3′,5′ phosphoric and acid benzyl esters on guinea-pig ventricular myocardium.” Naunyn-Schmiedeberg's Arch. Pharmacol. 310, 103-111; Kumar, A., et al (1990) “Synthesis and biological evaluation of some cyclic phosphoramidate nucleoside derivatives.” J. Med. Chem., 33, 2368-2375; LeBec, C., and Huynh-Dinh, T. (1991) “Synthesis of lipophilic phosphate triester derivatives of 5-fluorouridine an arabinocytidine as anticancer prodrugs.” Tetrahedron Lett. 32, 6553-6556; Lichtenstein, J., et al (1960) “The metabolism of exogenously supplied nucleotides by Escherichia coli, “J. Biol. Chem. 235, 457-465; Luethy, J., et al (1981) “Synthesis and toxicological properties of three naturally occurring cyanoepithioalkanes”. Mitt. Geg. Lebensmittelunters. Hyg. 72, 131-133 (Chem. Abstr. 95, 127093); McGigan, C. et al (1989) “Synthesis and biological evaluation of some phosphate triester derivatives of the anti-viral drug araA.” Nucleic Acids Res. 17, 6065-6075; McGuigan, C., et al (1990a) “Synthesis and evaluation of some novel phosphoramidate derivatives of 3′-azido-3′-deoxythymidine (AZT) as anti-HIV compounds.” Antiviral Chem. Chemother. 1107-113; McGuigan, C., et al (1990b) “Synthesis and anti-HIV activity of some novel substituted dialkyl phosphate derivatives of AZT and ddCyd.” Antiviral Chem. Chemother. 1, 355-360; McGuigan, C., et al (1990c) “Synthesis of some novel dialkyl phosphate derivative of 3′-modified nucleosides as potential anti-AIDS drugs.” Antiviral Chem. Chemother. 1, 25-33; McGuigan, C., et al (1991) “Synthesis and anti-HIV activity of some haloalkyl phosphoramidate derivatives of 3′-azido-3′-deoxythylmidine (AZT); potent activity of the trichloroethyl methoxyalaninyl compound.” Antiviral Res. 15, 255-263; McGuigan, C., et al (1993b) “Intracellular delivery of bioactive AZT nucleotides by aryl phosphate derivatives of AZT.” J. Med. Chem. 36, 1048-1052; Wagner, C. R., et al (2000) “Pronucleotides: towards the in vivo delivery of antiviral and anticancer nucleotides.” Med. Res. Rev. 20, 4 17-45 1; Meier, C. (2002) “cycloSal-pronucleotides-design of chemical Trojan horses.” Mini Rev. Med. Chem. 2, 219-234; Cahard, D., McGuigan, C. Balzarini, J. (2004a) “Aryloxy phosphoramidate trimesters as pro-tides.” Mini Rev. Med. Chem. 4, 371-381; Peyrottes, S., et al (2004b) “SATE pronucleotide approaches: an overview.” Mini Rev. Med. Chem. 4, 395-408; Drontle, D. P., Wagner, C. R. (2004c) “Design a pronucleotide stratagem: lessons from amino acid phosphoramidates of anticancer and antiviral pyrimidines.” Mini Rev. Med. Chem. 4, 409-419. Meyer, R. B., Jr., Shuman, D. A. and Robins, R. K. (1973) “Synthesis of purine nucleoside 3′,5′-cyclic phosphoramidates.” Tetrahedron Lett. 269-272; Nagyvary, J. et al (1973) “Studies on neutral esters of cyclic AMP,” Biochem. Biophys. Res. Commun. 55, 1072-1077; Namane, A. et al (1992) “Improved brain delivery of AZT using a glycosyl phosphotriester prodrug.” J. Med. Chem. 35, 3039-3044; Nargeot, J. et al (1983) PNAS 80, 2395-2399; Nelson, K. A, et al (1987) “The question of chair-twist equilibria for the phosphate rings of nucleoside cyclic 3′, 5′ monophosphates. ¹HNMR and x-ray crystallographic study of the diastereomers of thymidine phenyl cyclic 3′,5′-monophosphate.” J. Am. Chem. Soc. 109, 4058-4064; Nerbonne, J. M., et al (1984) “New photoactivatable cyclic nucleotides produce intracellular jumps in cyclic AMP and cyclic GMP concentrations.” Nature 301, 74-76; Neumann, J. M., et al (1989) “Synthesis and transmembrane transport studies by NMR of a glucosyl phospliolipid of tliyniidine.” J. Am. Chem. Soc. 111, 4270-4277; Ohno, R., et al (1991) “Treatment of myelodysplastic syndromes with orally administered 1-β-D-arabinouranosylcytosine-5′ stearylphosphate.” Oncology 48, 451-455. Palomino, E. et al (1989) “A dihydropyridine carrier system for sustained delivery of 2′, 3′ dideoxynucleosides to the brain.” J. Med. Chem. 32, 22-625; Perkins, R. M., et al (1993) “Activity of BRL47923 and its oral prodrug, SB203657A against a rauscher murine leukemia virus infection in mice.” Antiviral Res. 20 (Suppl. I). 84; Piantadosi, C., et al (1991) “Synthesis and evaluation of novel ether lipid nucleoside conjugates for anti-HIV-1 activity.” J. Med. Chem. 34, 1408-1414; Pompon, A., et al (1994). “Decomposition pathways of the mono- and bis(pivaloyloxymethyl) esters of azidothymidine-5′-monophosphate in cell extract and in tissue culture medium; an application of the on-line ISRP-cleaning HPLC technique.” Antiviral Chem. Chemother. 5, 91-98; Postemark, T. (1974) “Cyclic AMP and cyclic GMP.” Annu. Rev. Pharmacol. 14, 23-33; Prisbe, E. J. et al (1986) “Synthesis and antiherpes virus activity of phosphate and phosphonate derivatives of 9-[(1,3-dihydroxy-2-propoxy)methyl]guanine.” J. Med. Chem. 29, 671-675; Pucch, F. et al (1993) “Intracellular delivery of nucleoside monophosphate through a reductase-mediated activation process.” Antiviral Res. 22, 155-174; Pugaeva, V. P. et al (1969) “Toxicological assessment and health standard ratings for ethylene sulfide in the industrial atmosphere.” Gig TrJ: Prof Zabol 14, 47-48 (Chem. Abstr. 72, 212); Robins, R. K. (1984) “The potential of nucleotide analogs as inhibitors of Retro viruses and tumors.” Pharm. Res. 11-18; Rosowsky, A, et al (1982) “Lipophilic 5′-(alkylphosphate) esters of 1-β-D-arabinofuranosylcytosine and its N4-acyl and 2,2′-anhydro-3′-O-acyl derivatives as potential prodrugs.” J. Med. Chem. 25, 171-178; Ross, W. (1961) “Increased sensitivity of the walker turnout towards aromatic nitrogen mustards carrying basic side chains following glucose pretreatment.” Biochem. Pharm. 8, 235-240; Ryu, E. K., et al (1982). “Phospholipid-nucleoside conjugates. 3. Synthesis and preliminary biological evaluation of 1-β-D-arabinofuranosylcytosine 5′-diphosphate[-], 2-diacylglycerols.” J. Med. Chem. 25, 1322-1329; Saffhill, R. and Hume, W. J. (1986) “The degradation of 5-iododeoxyuridine and 5-bromoethoxyuridine by serum from different sources and its consequences for the use of these compounds for incorporation into DNA.” Chem. Biol. Interact. 57, 347-355; Saneyoshi, M. et al (1980) “Synthetic nucleosides and nucleotides. XVI. Synthesis and biological evaluations of a series of 1-β-D-arabinofuranosylcytosine 5′-alkyl or arylphosphates.” Chem. Pharm. Bull. 28, 2915-2923; Sastry, J. K. et al (1992) “Membrane permeable dideoxyuridine 5′-monophosphate analogue inhibits human immunodeficiency virus infection.” Mol. Pharmacol. 41, 441-445; Shaw, J. P. et al (1994) “Oral bioavailability of PMEA from PMEA prodrugs in male Sprague-Dawley rats.” 9th Annual AAPS Meeting. San Diego, Calif. (Abstract). Shuto, S. et al (1987) “A facile one-step synthesis of 5′ phosphatidylnucleosides by an enzymatic two-phase reaction.” Tetrahedron Lett. 28, 199-202; Shuto, S. et al (1988) Chem. Pharm. Bull. 36, 209-217. An example of a useful phosphate prodrug group is the S-acyl-2-thioethyl group, also referred to as “SATE”.

Similarly, the 5′-phosphonate can also be provided as various phosphonate prodrugs to increase the activity, bioavailability, stability or otherwise alter the properties of the phosphonate. A number of phosphonate prodrug ligands are known. In general, alkylation, acylation or other lipophilic modification of one or more hydroxy on the phosphonate moiety can be used to achieve a desired effect.

Nonlimiting examples of nucleotide prodrugs are described in the following references: J. K. Dickson, Jr. et al, “Orally active squalene synthetase inhibitors: bis((acyloxy)alkyl) prodrugs of the α-phosphonosulfonic acid moiety” J. Med. Chem. 1996, 39, 661-664; T. Kurz, et al, “Synthesis and antimalarial activity of chain substituted pivaloyloxymethyl ester analogues of Fosmidomycin and FR900098” Bioorg. Med. Chem. 2006, 14, 5121-5135; J. E. Starrett, Jr. et al, “Synthesis, oral bioavailability determination, and in vitro evaluation of prodrugs of the antiviral agent 9-[2-(phosphonomethoxy)ethyl]adenine (PMEA)” J. Med. Chem. 1994, 37, 1857-1864; H. T. Serafinowska, et al, “Synthesis and in vivo evaluation of prodrugs of 9-[2-(phosphonomethoxy)ethoxy]adenine” J. Med. Chem. 1995, 38, 1372-1379; S. Benzaria, et al, “Synthesis, in vitro antiviral evaluation, and stability studies of bis(S-acyl-2-thioethyl) ester derivatives of 9-[2-(phosphonomethoxy)ethyl]adenine (PMEA) as potential PMEA prodrugs with improved oral bioavailability” J. Med. Chem. 1996, 39, 4958-4965; M. S. Louie and H. Chapman, “An efficient process for the synthesis of cyclic HPMPC” Nucleosides, Nucleotides Nucleic acid 2001, 20, 1099-1102; J.-R. Choi, et al, “A novel class of phosphonate nucleosides. 9-[(1-phosphonomethoxy)-cyclopropyl)methyl]guanine as a potent and selective anti-HBV agent” J. Med. Chem. 2004, 47, 2864-2869; M. Wu, et al, “Synthesis of 9-[1-(substituted)-3-(phosphonomethoxy)propyl]adenine derivatives as possible antiviral agents” Nucleosides, Nucleotides Nucleic acid. 2005, 24, 1543-1568; X. Fu, et al, “Design and synthesis of novel bis(L-amino acid) ester prodrugs of 9-[2-(phosphonomethoxy)ethyl]adenine (PMEA) with improved anti-HBV activity” Bioorg. Med. Chem. Lett. 2007, 17, 465-470.

### Combination and Alternation Therapy for HIV, HBV or HCV

It has been recognized that drug-resistant variants of HIV, HBV and HCV can emerge after prolonged treatment with an antiviral agent. Drug resistance most typically occurs by mutation of a gene that encodes for a protein such as an enzyme used in viral replication, and most typically in the case of HIV, reverse transcriptase, protease, or DNA polymerase, and in the case of HBV, DNA polymerase, or in the case of HCV, RNA polymerase, protease, or helicase. Recently, it has been demonstrated that the efficacy of a drug against HIV infection can be prolonged, augmented, or restored by administering the compound in combination or alternation with a second, and perhaps third, antiviral compound that induces a different mutation from that caused by the principle drug. Alternatively, the pharmacokinetics, biodistribution, or other parameter of the drug can be altered by such combination or alternation therapy. In general, combination therapy is typically preferred over alternation therapy because it induces multiple simultaneous stresses on the virus.

The second antiviral agent for the treatment of HIV, in one embodiment, can be a reverse transcriptase inhibitor (a “RTI”), which can be either a synthetic nucleoside (a “NRTI”) or a non-nucleoside compound (a “NNRTI”). In an alternative embodiment, in the case of HIV, the second (or third) antiviral agent can be a protease inhibitor. In other embodiments, the second (or third) compound can be a pyrophosphate analog, or a fusion binding inhibitor. A list compiling resistance data collected in vitro and in vivo for a number of antiviral compounds is found in Schinazi, et al, Mutations in retroviral genes associated with drug resistance, International Antiviral News, 1997.

Preferred compounds for combination or alternation therapy for the treatment of HBV include 3TC, FTC, L-FMAU, interferon, adefovir dipivoxil, entecavir, telbivudine (L-dT), valtorcitabine (3′-valinyl L-dC), β-D-dioxolanyl-guanine (DXG), β-D-dioxolanyl-2,6-diaminopurine (DAPD), and β-D-dioxolanyl-6-chloropurine (ACP), famciclovir, penciclovir, lobucavir, ganciclovir, and ribavann.

Preferred examples of antiviral agents that can be used in combination or alternation with the compounds disclosed herein for HIV therapy include cis-2-hydroxymethyl-5-(5-fluorocytosin-1-yl)-1,3-oxathiolane (FTC); the (−)-enantiomer of 2-hydroxymethyl-5-(cytosin-1-yl)-1,3-oxathiolane (3TC); ziagen (abacavir), emtriva, viread (tenofovir DF), carbovir, acyclovir, foscarnet, interferon, AZT, DDI, D4T, CS-87 (3′-azido-2′,3′-dideoxyuridine), and β-D-dioxolane nucleosides such as β-D-dioxolanyl-guanine (DXG), β-D-dioxolanyl-2,6-diaminopurine (DAPD), and β-D-dioxolanyl-6-chloropurine (ACP), and integrase inhibitors such as MK-0518.

Preferred protease inhibitors (PIs) include crixivan (indinavir), viracept (nelfinavir), norvir (ritonavir), invirase (saquinavir), aptivus (tipranavir), kaletra, lexiva (fosamprenavir), reyataz (atazanavir) and TMC-114.

Preferred Non-Nucleoside Reverse Transcriptase Inhibitors (NNRTIs) include rescripton (delavirdine), sustiva (efavirenz), viramune (nevirapine) and TMC-125.

Preferred Entry inhibitors include fuzeon (T-20), PRO-542, TNX-355, vicriviroc, aplaviroc and maraviroc.

A more comprehensive list of compounds that can be administered in combination or alternation with any of the disclosed nucleosides include (1S,4R)-4-[2-amino-6-cyclopropylamino)-9H-purin-9-yl]-2-cyclopentene-1-methanol succinate (“1592”, a carbovir analog; GlaxoWellcome); 3TC: (−)-β-L-2′,3′-dideoxy-3′-thiacytidine (GlaxoWellcome); a-APA R18893: a-nitro-anilino-phenylacetamide; A-77003; C2 symmetry-based protease inhibitor (Abbott); A-75925: C2 symmetry-based protease inhibitor (Abbott); AAP-BHAP: bishetero-arylpiperazine analog (Upjohn); ABT-538: C2-symmetry-based protease inhibitor (Abbott); AzddU: 3′-azido-2′,3′-dideoxyuridine; AZT: 3′-azido-3′-deoxythymidine (GlaxoWellcome); AZT-p-ddI: 3′-azido-3′-deoxythymidilyl-(5′,5′)-2′,3′-dideoxyinosinic acid (Ivax); BHAP: bisheteroaryl-piperazine; BILA 1906: N-{1S-[[[3-[2S-{(1,1-dimethylethyl)amino]carbonyl}-4R]-3-pyridinylmethyl)thio]-1-piperidinyl]-2R-hydroxy-1S-(phenylmethyl)-propyl]amino]-carbonyl]-2-methylpropyl}-2-quinolinecarboxamide (Bio Mega/Boehringer-Ingelheim); BILA 2185: N-(1,1-dimethylethyl)-1-[2S-[[2-2,6-dimethyphenoxy)-1-xoethyl]amino]-2R-hydroxy-4-phenylbutyl]-4R-pyridinylthio)-2-piperidinecarboxamide (BioMega/Boehringer-Ingelheim); BMS186,318: aminodiol derivative HIV-1 protease inhibitor (Bristol-Myers-Squibb); d4API: 9-[2,5-d]hydro-5-(phosphonomethoxy)-2-furanyladenine (Gilead); d4C: 2′,3′-didehydro-2′,3′-dideoxycytidined; d4T: 2′,3′-didehydro-3′-deoxythymidine (Bristol-Myers-Squibb); ddC; 2′,3′-dideoxycytidine (Roche); ddI: 2′,3′-dideoxyinosine (Bristol-Myers-Squibb); DMP-266: a 1,4-dihydro-2H-3,1-benzoxazin-2-one; DMP-450: {[4R-(4-a,5-a,6-b,7-b)]-hexahydro-5,6-bis(hydroxy)-1,3-bis(3-amino)phenyl]-methyl)-4,7-bis-(phenylmethyl)-2H-1,3-diazepin-2-one}-bismesylate (Gilead); DXG: (−)-β-D-dioxolane-guanosine (Gilead); EBU-dM: 5-ethyl-1-ethoxymethyl-6-(3,5-dimethylbenzyl)-uracil; E-EBU: 5-ethyl-1-ethoxymethyl-6-benzyluracil; DS: dextran sulfate; E-EPSeU: 1-(ethoxymethyl)-(6-phenylselenyl)-5-ethyluracil; E-EPU: 1-(ethoxymethyl)-(6-phenylthio)-5-ethyluracil; FTC: β-2′,3′-dideoxy-5-fluoro-3′-thiacytidine (Gilead); HBY097: S-4-isopropoxycarbonyl-6-methoxy-3-(methylthio-methyl)-3,4-dihydroquinoxalin-2(1H)-thione; HEPT: 1-[(2-hydroxyethoxy)methyl]-6-(phenylthio)thymine; HIV-1: human immunodeficiency virus type 1; JM2763: 1,1′-(1,3-propanediyl)-bis-1,4,8,11-tetraaza-cyclotetradecane (Johnson Matthey); JM3100: 1,1′-[1,4-phenylenebis-(methylene)]-bis-1,4,8,11-tetraazacyclotetradecane (Johnson Matthey); KNI-272: (2S,3S)-3-amino-2-hydroxy-4-phenylbutyric acid-containing tripeptide; L-697,593; 5-ethyl-6-methyl-3-(2-phthalimido-ethyl)pyridin-2(1H)-one; L-735,524: hydroxy-amino-pentane amide HIV-1 protease inhibitor (Merck); L-697,661: 3-{[(4,7-dichloro-1,3-benzoxazol-2-yl)methyl]amino}-5-ethyl-6-methylpyridin-2(1H)-one; L-FDDC: (−)-β-L-5-fluoro-2′,3′-dideoxycytidine; L-FDOC: (−)-β-L-5-fluoro-dioxolane cytosine; MKC442: 6-benzyl-1-ethoxymethyl-5-isopropyluracil (1-EBU; Mitsubishi); Nevirapine: 11-cyclopropyl-5,11-dihydro-4-methyl-6H-dipyridol-[3,2-b:2′,3′-e]-diazepin-6-one (Boehringer-Ingelheim); NSC648400: 1-benzyloxymethyl-5-ethyl-6-(alpha-pyridylthio)uracil (E-BPTU); P9941: [2-pyridylacetyl-IlePheAla-y(CHOH)]₂ (Dupont Merck); PFA: phosphonoformate (foscarnet; Astra); PMEA: 9-(2-phosphonylmethoxyethyl)adenine (Gilead); PMPA: (R)-9-(2-phosphonylmethoxypropyl)adenine (Gilead); Ro 31-8959: hydroxyethylamine derivative HIV-1 protease inhibitor (Roche); RPI-312: peptidyl protease inhibitor, 1-[(3S)-3-(n-alpha-benzyloxycarbony 1)-1-asparginyl)-amino-2-hydroxy-4-phenylbutyryl]-n-tert-1-proline amide; 2720: 6-chloro-3,3-dimethyl-4-(isopropenyloxycarbonyl)-3,4-dihydro-quinoxalin-2-(1H)-thione; SC-52151: hydroxy-ethylurea isostere protease inhibitor (Searle); SC-55389A: hydroxyethyl-urea isostere protease inhibitor (Searle); TIBO R82150: (+)-(5S)-4,5,6,7-tetrahydro-5-methyl-6-(3-methyl-2-butenyl)-imidazo[4,5,1-jk]-[1,4]benzodiazepin-2(1H)-thione (Janssen); TIBO 82913: (+)-(5s)-4,5,6,7,-tetrahydro-9-chloro-5-methyl-6-(3-methyl-2-butenyl)imidazo[4,5,1jk]-[1,4]benzo-diazepin-2(1H)-thione (Janssen); TSAO-m3T: [2′,5′-bis-O-(tert-butyl-dimethylsilyl)-3′-spiro-5′-(4′-amino-1′,2′-oxathiole-2′,2′-dioxide)]-β-D-pento-furanosyl-N-3-methylthymine; U90152: 1-[3-[(1-methylethyl)-amino]-2-pyridinyl]-4-[[5-[(methylsulphonyl)-amino]-1H-indol-2-yl]carbonyl]piperazine; UC: thiocarboxanilide derivatives (Uniroyal); UC-781: N-[4-chloro-3-(3-methyl-2-butenyloxy)phenyl]-2-methyl-3-furancarbothioamide; UC-82: N-[4-chloro-3-(3-methyl-2-butenyloxy)phenyl]-2-methyl-3-thiophenecarbothioamide; VB 11,328: hydroxyethyl-sulphonamide protease inhibitor (Vertex); VX-478: hydroxyethylsulphonamide protease inhibitor (Vertex); XM 323: cyclic urea protease inhibitor (Dupont Merck).

The active compound can also be administered in combination or alternation with ribavarin, interferon, interleukin or a stabilized prodrug of any of them. More broadly described, the compound can be administered in combination or alternation with any of the anti-HCV drugs listed below.

Table of anti-Hepatitis C Compounds in Current Clinical Development

| | | Pharmaceutical |
|---|---|---|
| Drug name | Drug category | Company |
| PEGASYS | Long acting interferon | Roche |
| pegylated interferon | | |
| alfa-2a | | |
| INFERGEN | Long acting interferon | InterMune |
| interferon alfacon-1 | | |
| OMNIFERON | Long acting interferon | Viragen |
| natural interferon | | |
| ALBUFERON | Long acting interferon | Human Genome |
| | | Sciences |
| REBIF | Interferon | Ares-Serono |
| interferon beta-la | | |
| Omega Interferon | Interferon | BioMedicine |
| Oral Interferon alpha | Oral Interferon | Amarillo Biosciences |
| Interferon gamma-lb | Anti-fibrotic | InterMune |
| IP-501 | Anti-fibrotic | InterMune |
| Merimebodib VX-497 | IMPDH inhibitor | Vertex |
| | (inosine monophosphate | |
| | dehydrogenase) | |
| AMANTADINE | Broad Antiviral Agent | Endo Labs |
| (Symmetrel) | | Solvay |
| IDN-6556 | Apotosis regulation | Idun Pharma. |
| XTL-002 | Monclonal Antibody | XTL |
| HCV/MF59 | Vaccine | Chiron |
| CIVACIR | Polyclonal Antibody | NABI |
| | Therapeutic vaccine | Innogenetics |
| VIRAMIDINE | Nucleoside Analogue | ICN |
| ZADAXIN | Immunomodulator | Sci Clone |
| (thymosin alfa-1) | | |
| CEPLENE (histamine) | Immunomodulator | Maxim |
| VX 950/LY 570310 | Protease inhibitor | Vertex/Eli Lilly |
| ISIS 14803 | Antisense | Isis Pharmaceutical/ |
| | | Elan |
| IDN-6556 | Caspase inhibitor | Idun Pharmaceuticals |
| JTK 003 | Polymerase Inhibitor | AKROS Pharma |
| Tarvacin | Anti-Phospholipid | Peregrine |
| | Therapy | |
| HCV-796 | Polymerase Inhibitor | ViroPharma/Wyeth |
| CH-6 | Protease inhibitor | Schering |
| ANA971 | Isatoribine | ANADYS |
| ANA245 | Isatoribine | ANADYS |
| CPG 10101 (Actilon) | Immunomodulator | Coley |
| Rituximab (Rituxam) | Anti-CD2O | Genetech/IDEC |
| | Monoclonal Antibody | |
| NM283 | Polymerase Inhibitor | Idenix Pharmaceuticals |
| (Valopicitabine) | | |
| HepX ™-C | Monoclonal Antibody | XTL |
| IC41 | Therapeutic Vaccine | Intercell |
| Medusa Interferon | Longer acting interferon | Flame1 Technologies |
| E-1 | Therapeutic Vaccine | Innogenetics |
| Multiferon | Long Acting Interferon | Viragen |
| BILN 2061 | Serine Protease inhibitor | Boehringer-Ingelheim |
| TMC435350 | Serine Protease inhibitor | Tibotec |
| Boceprevir | Serine Protease inhibitor | Schering-Plough |
| (SCH 503034) | | |
| nitazoxanide | To be determined | Romark |
| R7128/PSI6130 | Polymerase Inhibitor | Roche/Pharmasset |
| IDX184 | Polymerase Inhibitor | Idenix |
| R1626 | Polymerase inhibitor | Roche |
| MK-7009 | protease inhibitor | Merck |
| ITMN-191 | protease inhibitor | InterMune |
| Debio 025 | Cyclophilin inhibitor | Debiopharm |

### Combination Therapy for the Treatment of Proliferative Conditions

In another embodiment, the compounds, when used as an antiproliferative, can be administered in combination with another compound that increases the effectiveness of the therapy, including but not limited to an antifolate, a 5-fluoropyrimidine (including 5-fluorouracil), a cytidine analogue such as β-L-1,3-dioxolanyl cytidine or β-L-1,3-dioxolanyl 5-fluorocytidine, antimetabolites (including purine antimetabolites, cytarabine, fudarabine, floxuridine, 6-mercaptopurine, methotrexate, and 6-thioguanine), hydroxyurea, mitotic inhibitors (including CPT-11, Etoposide (VP-21), taxol, and vinca alkaloids such as vincristine and vinblastine, an alkylating agent (including but not limited to busulfan, chlorambucil, cyclophosphamide, ifofamide, mechlorethamine, melphalan, and thiotepa), nonclassical alkylating agents, platinum containing compounds, bleomycin, an anti-tumor antibiotic, an anthracycline such as doxorubicin and dannomycin, an anthracenedione, topoisomerase TI inhibitors, hormonal agents (including but not limited to corticosteroids (dexamethasone, prednisone, and methylprednisone), androgens such as fluoxymesterone and methyltestosterone, estrogens such as diethylstilbesterol, antiestrogens such as tamoxifen, LHRH analogues such as leuprolide, antiandrogens such as flutamide, aminoglutethimide, megestrol acetate, and medroxyprogesterone), asparaginase, carmustine, lomustine, hexamethyl-melamine, dacarbazine, mitotane, streptozocin, cisplatin, carboplatin, levamasole, and leucovorin. The compounds of the present invention can also be used in combination with enzyme therapy agents and immune system modulators such as an interferon, interleukin, tumor necrosis factor, macrophage colony-stimulating factor and colony stimulating factor.

Although the invention has been described with respect to various preferred embodiments, it is not intended to be limited thereto, but rather those skilled in the art will recognize that variations and modifications may be made therein which are within the spirit of the invention and the scope of the appended claims.

### Abbreviations

Abbreviations which may be used in the descriptions of the scheme and the examples that follow are:
Ac for acetyl;
AcOH for acetic acid;
AIBN for azobisisobutyronitrile;
BINAP for 2,2′-bis(diphenylphosphino)-1,1′-binaphthyl;
Boc₂O for di-tert-butyl-dicarbonate;
Boc for t-butoxycarbonyl;
Bpoc for 1-methyl-1-(4-biphenylyl)ethyl carbonyl;
Bz for benzoyl;
Bn for benzyl;
BocNHOH for tert-butyl N-hydroxycarbamate;
t-BuOK for potassium tert-butoxide;
Bu₃SnH for tributyltin hydride;
BOP for (benzotriazol-1-yloxy)tris(dimethylamino)phosphonium Hexafluorophosphate;
Brine for sodium chloride solution in water;
CDI for carbonyldiimidazole;
CH₂Cl₂ for dichloromethane;
CH₃ for methyl;
CH₃CN for acetonitrile;
Cs₂CO₃ for cesium carbonate;
CuCl for copper (I) chloride;
CuI for copper (I) iodide;
dba for dibenzylidene acetone;
dppb for diphenylphosphino butane;
DBU for 1,8-diazabicyclo[5.4.0]undec-7-ene;
DCC for N,N′-dicyclohexylcarbodiimide;
DEAD for diethylazodicarboxylate;
DIAD for diisopropyl azodicarboxylate;
DIPEA or (i-Pr)₂EtN for N,N,-diisopropylethyl amine;
Dess-Martin periodinane for 1,1,1-tris(acetyloxy)-1,1-dihydro-1,2-benziodoxol-3-(1H)-one;
DMAP for 4-dimethylaminopyridine;
DME for 1,2-dimethoxyethane;
DMF for N,N-dimethylformamide;
DMSO for dimethyl sulfoxide;
DMT for di(p-methoxyphenyl)phenylmethyl or dimethoxytrityl
DPPA for diphenylphosphoryl azide;
EDC for N-(3-dimethylaminopropyl)-N′-ethylcarbodiimide;
EDC HCl for N-(3-dimethylaminopropyl)-N′-ethylcarbodiimide hydrochloride;
EtOAc for ethyl acetate;
EtOH for ethanol;
Et₂O for diethyl ether;
HATU for O-(7-azabenzotriazol-1-yl)-N,N,N′,N′,-tetramethyluronium Hexafluorophosphate;
HCl for hydrogen chloride;
HOBT for 1-hydroxybenzotriazole;
K₂CO₃ for potassium carbonate;
n-BuLi for n-butyl lithium;
i-BuLi for i-butyl lithium;
t-BuLi for t-butyl lithium;
PhLi for phenyl lithium;
LDA for lithium diisopropylamide;
TMEDA for N,N,N′,N′-tetramethylethylenediamine;
LiTMP for lithium 2,2,6,6-tetramethylpiperidinate;
MeOH for methanol;
Mg for magnesium;
MOM for methoxymethyl;
Ms for mesyl or —SO₂—CH₃;
Ms₂O for methanesulfonic anhydride or mesyl-anhydride;
NaN(TMS)₂ for sodium bis(trimethylsilyl)amide;
NaCl for sodium chloride;
NaH for sodium hydride;
NaHCO₃ for sodium bicarbonate or sodium hydrogen carbonate;
Na₂CO₃ sodium carbonate;
NaOH for sodium hydroxide;
Na₂SO₄ for sodium sulfate;
NaHSO₃ for sodium bisulfite or sodium hydrogen sulfite;
Na₂S₂O₃ for sodium thiosulfate;
NH₂NH₂ for hydrazine;
NH₄HCO₃ for ammonium bicarbonate;
NH₄Cl for ammonium chloride;
NMMO for N-methylmorpholine N-oxide;
NaIO₄ for sodium periodate;
Ni for nickel;
OH for hydroxyl;
OsO₄ for osmium tetroxide;
TEA or Et₃N for triethylamine;
TFA for trifluoroacetic acid;
THF for tetrahydrofuran;
TPP or PPh₃ for triphenylphosphine;
Troc for 2,2,2-trichloroethyl carbonyl;
Ts for tosyl or —SO₂—C₆H₄CH₃;
Ts₂O for tolylsulfonic anhydride or tosyl-anhydride;
TsOH for p-tolylsulfonic acid;
Pd for palladium;
Ph for phenyl;
POPd for dihydrogen dichlorobis(di-tert-butylphosphinito-κP)palladate(II);
Pd₂(dba)₃ for tris(dibenzylideneacetone) dipalladium (0);
Pd(PPh₃)₄ for tetrakis(triphenylphosphine)palladium (0);
PdCl₂(Ph₃P)₂ for trans-dichlorobis(triphenylphosphine)palladium (II);
Pt for platinum;
Rh for rhodium;
Ru for ruthenium;
TBS for tert-butyl dimethylsilyl;
TMS for trimethylsilyl; or
TMSCl for trimethylsilyl chloride.

### Synthetic Methods

The compounds and processes of the present invention will be better understood in connection with the following synthetic schemes that illustrate the methods by which the compounds of the invention may be prepared. These schemes are of illustrative purpose, and are not meant to limit the scope of the invention. Equivalent, similar, or suitable solvents, reagents or reaction conditions may be substituted for those particular solvents, reagents, or reaction conditions described herein without departing from the general scope of the method of synthesis.

The syntheses of 2′-fluoronucleosides (Scheme 1, 1-1) have been well documented in the literature, see references cited in a review article by K. W. Pankiewicz, Carbohydrate Research, 2000, 327, 87-105 and nonlimiting examples of process: Clark et al, J. Med. Chem. 2005, 48, 5504; Clark et al, Bioorg. Med. Chem. Lett. 2006, 16, 1712; Clark et al, J. Carbohydr. Chem. 2006, 25, 461; Seela et al, Org. Biomol. Chem. 2008, 6, 596; Pan et al, J. Org. Chem. 1999, 94, 4; Shi et al, Bioorg. Med. Chem. 2005, 13, 1641; He et al, J. Org. Chem. 2003, 68, 5519; Gudmundsson et al, J. Med. Chem. 2000, 43, 2473; and Jean-Baptiste et al, Synlett 2008, 817. Nucleoside (1-1) can be converted to 3′-O-benzoyl protected 5′-aldehyde (1-2) through four steps: 1) selective protection of 5′-OH with tert-butyl diphenylsilyl chloride; 2) protection of 3′-OH with benzoyl chloride; 3) releasing 5′-OH with a fluoride; and 4) oxidation of 5′-OH to 5′-aldehyde with an oxidant such as Dess-Martin Periodinane or DMSO-DCC. The aldehyde (1-2) then serves as a universal intermediate to prepare phosphonates (I-1˜I-6).

The aldehyde (1-2) can be transformed to olefin (1-3) through a Horner-Wadsworth-Emmons reaction using the conditions elaborated by Xu et al (Org. Lett. 2003, 5, 2267). The olefin (1-3) can be saturated with a reducing agent such as diimide, NaBH₄ or the like, or through various hydrogenation conditions: hydrogen under a metallic catalyst such as palladium, platinum or the like, to intermediate (1-4), which is then converted to compound (I-1) after deprotection. Similarly (I-2) is obtained from compound (1-3) after de-protection. In another route, the aldehyde group of (1-2) can be nucleophilically attacked by a suitable nucleophile such as diethyl (lithiodifluoromethyl)phosphonate (Obayashi et al, Tetrahedron Lett. 1982, 23, 2323) to give alcohol (1-5), which can be further converted to phosphonate (I-3) after deprotection. In another process, phosphonate (I-4) can be synthesized from alcohol (1-5) through Barton deoxygenation (using the procedure developed by Nieschalk et al, Tetrahedron 1996, 52, 165) followed by deprotection. In yet another process, phosphonate (I-5) can be synthesized from alcohol (1-5) through oxidation followed by deprotection. In yet another process, phosphonate (I-6) can be synthesized from alcohol (1-5) through fluorination followed by deprotection.

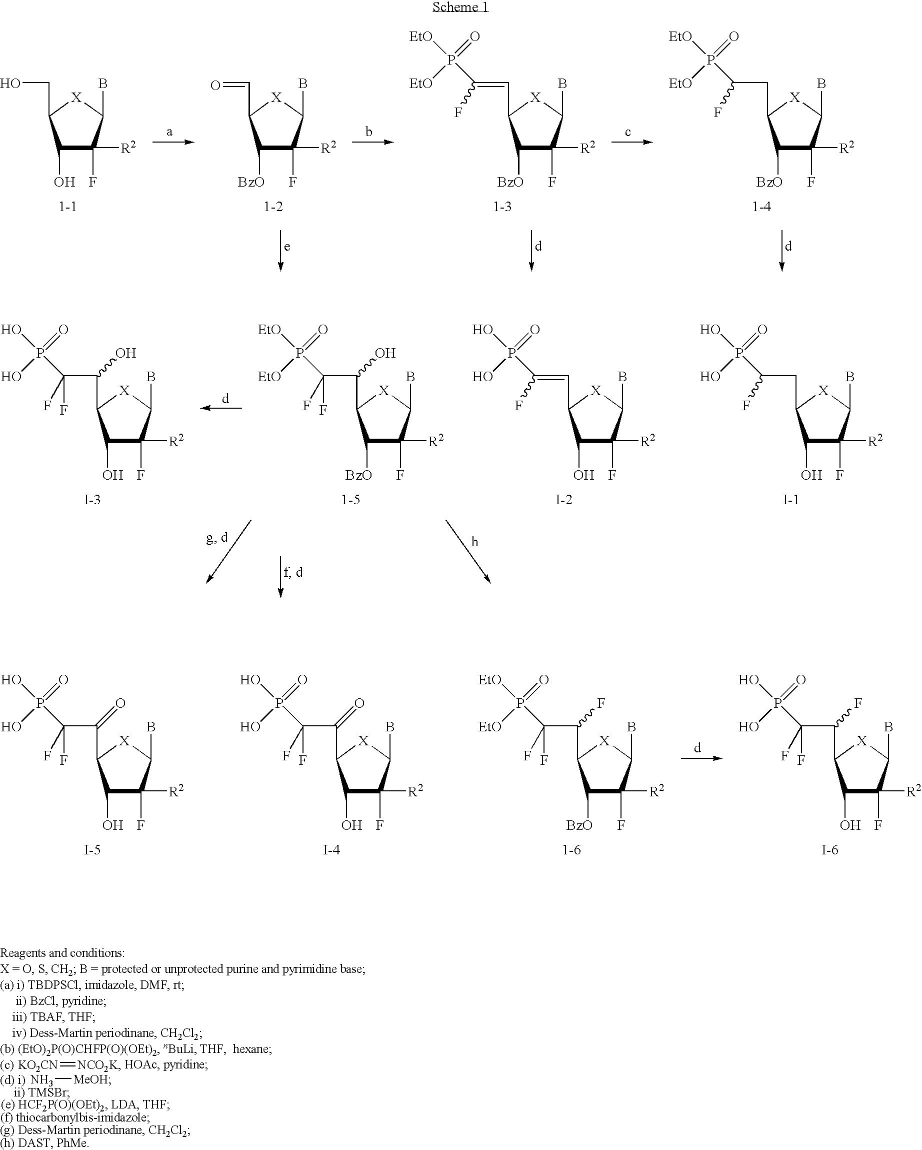

Alternatively, the compounds of the present invention can be prepared according to the procedures described in Scheme 2, as exemplified by the synthesis of fluorophosphonate (I-7). 3′,5′-Diol of nucleoside (2-1) can be selectively protected as a cyclic disilyl ether, while the 2′-OH can be oxidized to compound (2-2). The 2′-ketone of intermediate (2-2) can be attacked by methyl lithium, followed by desilylation with a fluoride to give triol (2-3). Manipulation through selective protection of 5′-OH and 3′-OH of (2-3) provides (2-4), which can be transformed to aldehyde (2-5) after removal of 5′-ODMT and oxidation of the released 5′-OH. The aldehyde (2-5) can be further converted to phosphonates (2-6) and (2-7) by similar procedures described in Scheme 1. The target (I-7) can be obtained from (2-7) through DAST fluorination and de-protections.

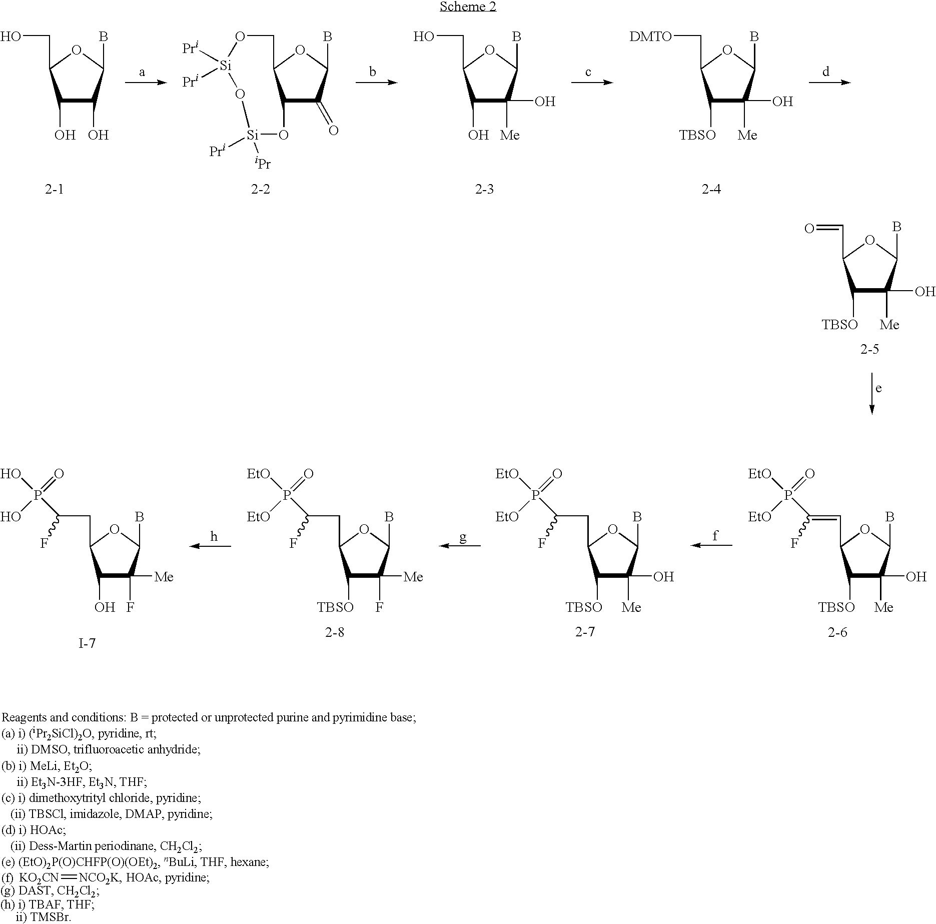

Alternatively, the compounds of the present invention can be prepared according to the procedures described in Scheme 3, as illustrated by the synthesis of fluorophosphonates (I-1 and I-4). The intermediate (3-1) can be obtained from nucleoside (1-1) through the procedures described in Scheme 1. The 5′-OH of nucleoside (3-1) can be activated to a leaving group (LG, such as —OMs or —OTf or the like) in (3-2), which can be further reacted by displacement with a suitable nucleophile such as the α-lithiated-α-fluorotrimethylsilyl-methylphosphonate carbanion (as elaborated by Nieschalk et al, Tetrahedron, 1996, 52, 165) or diethyl (lithiodifluoromethyl)phosphonate carbanion (as described in Scheme 1) to give compounds (1-4 and 3-3). The latter can be further converted to phosphonates (I-1 and I-4) after deprotection.

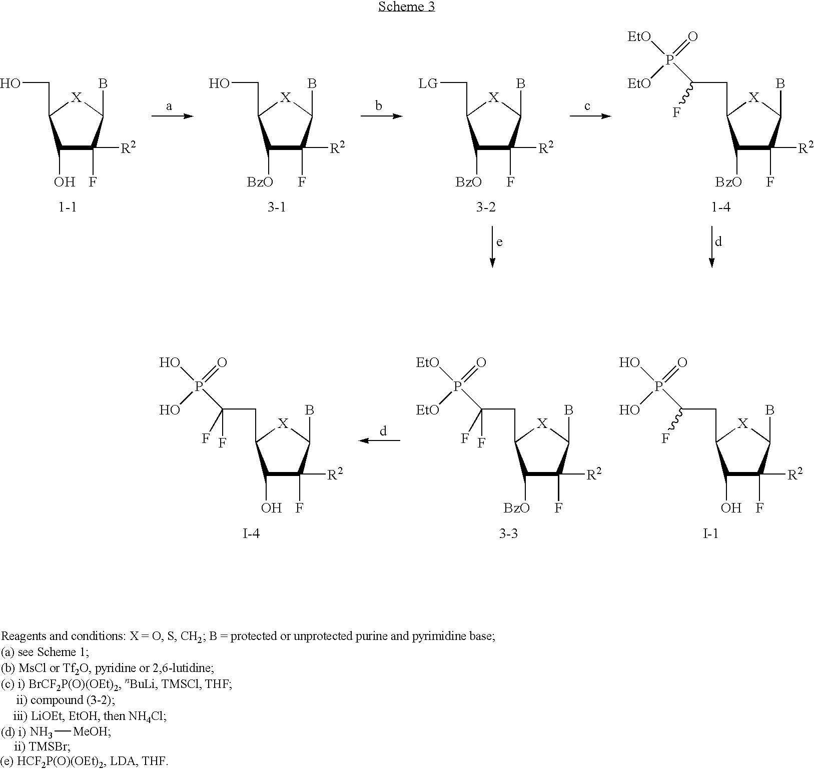

Alternatively, the compounds of the present invention may be prepared according to the procedures described in Scheme 4, as exemplified by the synthesis of fluorophosphonates (I-7) from various carbohydrate starting materials. The fluorinated ribosylfuanose (4-1, available from xylose, see Clark et al, J. Carbohydrate Chem. 2006, 25, 461) can be converted to (4-4) via intermediates (4-2) and (4-3) using similar procedures described in Schemes 1 and 3. The acetal of (4-4) can be selectively hydrolyzed and further acylated to (4-5), which can react with a silylated base under Vorbruggen condensation condition (see also Harry-O'kuru et al, J. Org. Chem. 1997, 62, 1754) to give the nucleoside phosphonate (4-6). The latter is further de-protected to provide the target phosphonate (I-7).

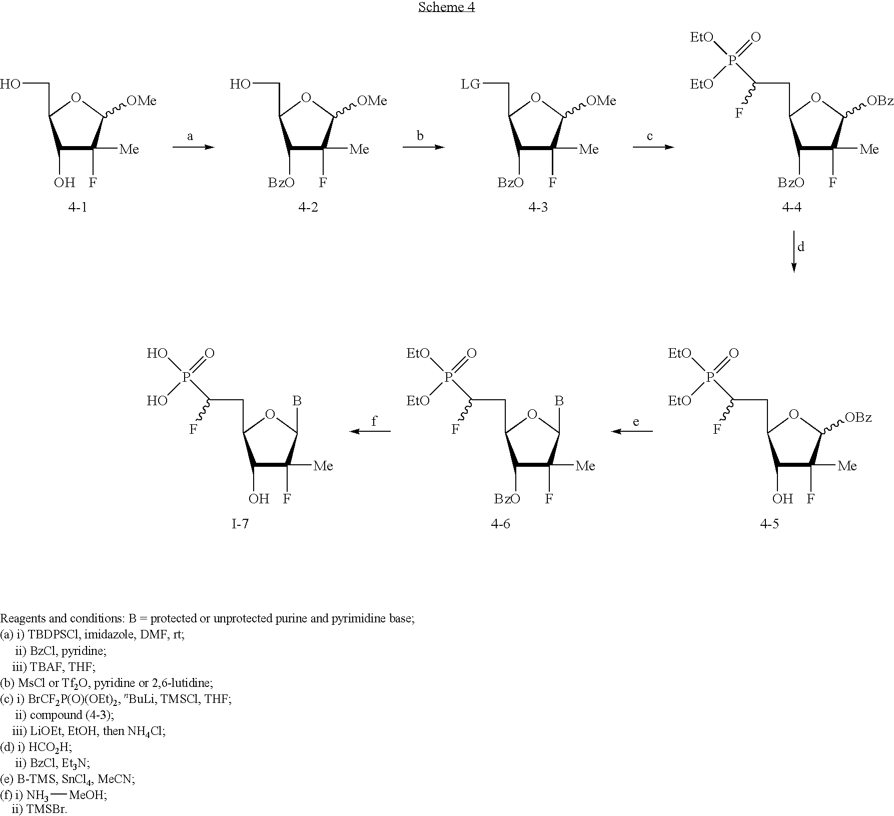

As shown in Scheme 5, compounds such as (I-8) and (II-1) of the present invention may be prepared from nucleoside (3-1). The 5′-OH of (3-1) can be converted to 5′-azido using the condition elaborated by Bevilacqua et al, J. Am. Chem. Soc. 1993, 115, 4985. The 5′-azide (5-1) can be reductively phosphorylated using the Staudinger reaction as elaborated by Lin et al, Synthetic Commun. 2000, 30, 1233; followed by de-protections to give the target compound (II-1). The conversion from the 5′-alcohol (3-1) to olefin (5-2) is similar to that described in Scheme 1. The cyclopropanation of (5-2) can be realized through a two-step procedure elaborated by Robins et al, Tetrahedron Lett. 1994, 35, 3445; while the epoxidation of (5-2) can be done by a dioxirane using an in situ procedure developed by Cristau et al, J. Organomet. Chem. 1998, 571, 189. Further reaction of (5-3) by de-protection afford the compound (I-8).

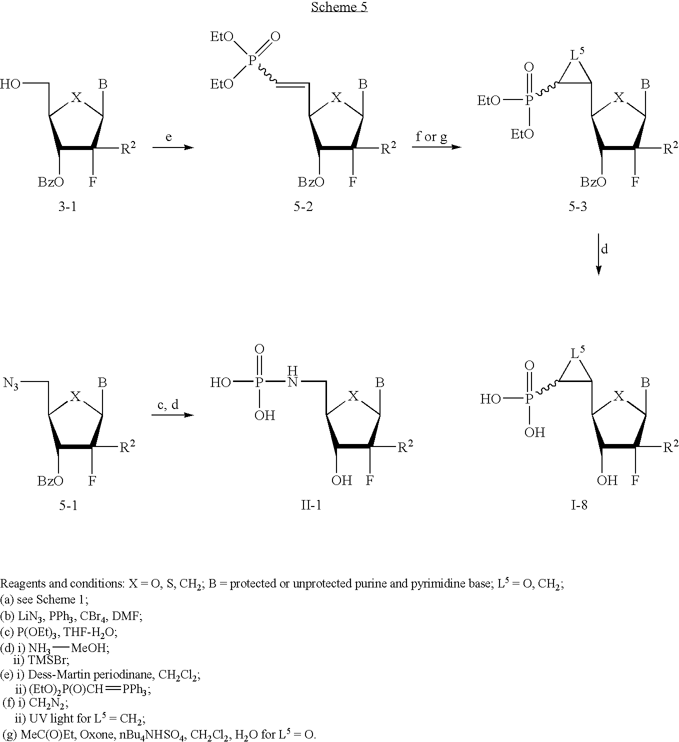

It will be appreciated that, with appropriate manipulation and protection of any chemical functionality, synthesis of compounds of Formula (I and II) is accomplished by methods analogous to those above and to those described in the Experimental section. Suitable protecting groups can be found, but are not restricted to, those found in T W Greene and P G M Wuts “Protective Groups in Organic Synthesis”, 3rd Ed (1999), J Wiley and Sons.

All references cited herein, whether in print, electronic, computer readable storage media or other form, are expressly incorporated by reference in their entirety, including but not limited to, abstracts, articles, journals, publications, texts, treatises, internet web sites, databases, patents, and patent publications.

### EXAMPLES

The compounds and processes of the present invention will be better understood in connection with the following examples, which are intended as an illustration only and not limiting of the scope of the invention. Various changes and modifications to the disclosed embodiments will be apparent to those skilled in the art and such changes and modifications including, without limitation, those relating to the chemical structures, substituents, derivatives, formulations and/or methods of the invention may be made without departing from the spirit of the invention and the scope of the appended claims.

### Example 1

### Compound of Formula (I), Wherein Base is N⁴-benzoylcytosin-1-yl, X is O, L² is CF₂, L¹ is CH₂, L³ is Absent, R¹═R³═R⁴═H, R²=Me, R^{3a}═OAc, W¹═W²═OEt

Step 1a. Into a solution of (3R,4S,5R)-3-fluoro-4-hydroxy-5-hydroxymethyl-3-methyldihydrofuran-2-one (800 mg, 4.9 mmol, prepared according to WO2006/031725 A2) in THF (16 mL) was added p-methoxybenzyl trichloroacetimidate (1.45 g, 5.0 mmol) and camphorsulfonic acid (341 mg, 1.47 mmol). The resulting mixture was stirred at room temperature for 3 hours before being partitioned (aqueous NaHCO₃-EtOAc). The organics were washed (aqueous NaHCO₃), dried (Na₂SO₄) and evaporated. The residue was chromatographed (silica, hexane-EtOAc) to give the desired compound (911 mg, 67%). ¹H NMR (CDCl₃) 7.26 (d, 2H), 6.89 (d, 2H), 4.53 (d, 2H), 4.40 (m, 1H), 4.14 (m, 2H), 3.83 (s, 3H), 3.76 (m, 1H), 1.64 (d, 3H).

Step 1b. Into a solution of compound from step 1a (910 mg, 3.20 mmol) in CH₂Cl₂ (6 mL) was added TMSCl (785 mg, 5.2 mmol), Et₃N (701 mg, 6.9 mmol) and DMAP (212 mg, 1.8 mmol). The resulting mixture was stirred at room temperature for 15 hours before being partitioned (water and EtOAc). The organics were washed (water), dried (Na₂SO₄) and evaporated. The residue was chromatographed (silica, hexane-EtOAc) to give the desired compound (770 mg, 61%). ¹HNMR (CDCl₃) 7.17 (d, 2H), 6.82 (d, 2H), 4.42 (s, 2H), 4.34 (m, 1H), 4.09 (m, 1H), 3.74 (s, 3H), 3.72 (d, 1H), 3.56 (d, 1H), 1.51 (d, 3H), 0.83 (s, 9H), 0.05 (s, 3H), 0.00 (s, 3H).

Step 1c. Into a solution of compound from step 1b (770 mg, 1.93 mmol) in THF (30 mL) was added lithium tri-tert-butoxyaluminum hydride (1M in THF, 4 mL) at −20° C. The resulting mixture was slowly warmed to 0° C. and was stirred at that temperature for 2 hours before being added aqueous sodium/potassium tartrate. The mixture was diluted with EtOAc and the organics was washed (aqueous NaHCO₃), dried (Na₂SO₄) and evaporated. The residue was chromatographed (silica, hexane-EtOAc) to give the desired compound (578 mg, 75%). ¹H NMR (CDCl₃) 7.26 (d, 2H), 6.91 (d, 2H), 5.05 (t, 1H), 4.60 (d, 1H), 4.51 (d, 1H), 4.23 (dd, 1H), 4.11 (m, 1H), 3.83 (s, 3H), 3.67 (d, 1H), 3.52 (d, 1H), 3.25 (d, 1H), 1.43 (d, 3H), 0.90 (s, 9H), 0.05 (s, 3H), 0.00 (s, 3H).

Step 1d. Into a solution of compound from step 1c (3.15 g, 7.88 mmol) in methyl iodide (13.4 mL) was added NaOH (50% in water, 27.5 mL) and tetrabutylammonium iodide (210 mg, 0.5 mmol). The resulting mixture was stirred at room temperature for 14 hours before being diluted with water and EtOAc. The organic phase was washed with water, dried (Na₂SO₄) and evaporated. The residue was chromatographed (silica, hexane-EtOAc) to give a colorless liquid, which was redissolved in 222 mL of CH₂Cl₂. The solution was then added aqueous buffer (22.2 mL, pH=7, dibasic/monobasic sodium phosphate) and 2,3-dichloro-5,6-dicyano-1,4-benzoquinone (3.7 g, 16.3 mmol). The resulting mixture was stirred at room temperature for 45 minutes before being partitioned between aqueous NaHCO₃ and EtOAc. The organic phase was washed with aqueous NaHCO₃, dried (Na₂SO₄) and evaporated. The residue was chromatographed (silica, hexane-EtOAc) to give the desired compound (2.10 g, 91% over 2 steps). ¹H NMR (CDCl₃) 4.74 (s, 1H), 4.08 (m, 1H), 3.95 (m, 1H), 3.93 (m, 1H), 3.64 (m, 1H), 3.52 (s, 3H), 1.71 (m, 1H), 1.44 (d, 3H), 0.94 (s, 9H), 0.14 (s, 3H), 0.12 (s, 3H).

Step 1e. Into a solution of compound from step 1d (110 mg, 0.37 mmol) in CH₂Cl₂ (10 mL) was added 2,6-lutidine and triflic anhydride (157 mg, 0.55 mmol) at −78° C. After 5 minutes, the resulting mixture was quenched with aqueous NaHCO₃ before being partitioned between water and EtOAc. The organic phase was washed with 1 M HCl, dried (Na₂SO₄) and evaporated. The residue was chromatographed (silica, hexane-EtOAc) to provide a colorless oil, which was redissolved in THF (3 mL) and was added into a solution of lithium diisopropylamide (1.11 mmol) and hexamethylphosphoramide (199 mg, 1.11 mmol) in THF (3.5 mL) at −78° C. The resulting mixture was stirred at −78° C. for 10 minutes before being diluted with water and EtOAc. The organic phase was washed with brine, dried (Na₂SO₄) and evaporated. The residue was chromatographed (silica, hexane-EtOAc) to give the desired compound (158 mg, 92%). ESIMS m/z=487.06 [M+Na]⁺.

Step 1f. Into a solution of compound from step 1e (132 mg, 0.28 mmol) in CH₂Cl₂ (3 mL) was added acetic anhydride (0.45 mL) and concentrated sulfuric acid (0.04 mL, 98%). The resulting mixture was stirred at room temperature for 1 hour before being partitioned between aqueous NaHCO₃ and EtOAc. The organic phase was washed with aqueous NaHCO₃, dried (Na₂SO₄) and evaporated. The residue was chromatographed (silica, hexane-EtOAc) to give the desired compound as a mixture of diastereomers (74 mg, 62%). ESIMS m/z=442.78 [M+Na]⁺.

Step 1g. N⁴-benzoylcytosine (0.29 mmol) and ammonium sulfate (1 mg) in hexamethyldisilazane (3 ml) was stirred at reflux for 1 hour before all volatiles were removed by rotavap. The resulting residue was redissolved in chlorobenzene (4 mL) and was added tin(II) chloride (149 mg, 0.57 mmol) and the compound from step 1f (60 mg, 0.143 mmol). The reaction mixture was stirred at 65° C. for 15 hours before being partitioned between water and EtOAc. The organic phase was washed with water, dried (Na₂SO₄) and evaporated. The residue was chromatographed (silica, hexane-EtOAc) to give the title compound (36 mg, 44%). ESIMS m/z=575.81 [M+H]⁺.

### Example 2

### Compound of Formula (I), Wherein Base is cytosine-1-yl, X is O, L² is CF₂, L¹ is CH₂, L³ is Absent, R¹═R³═R⁴═H, R²=Me, R^{3a}═OH, W¹═W²═OEt

The compound from step 1g (8 mg, 0.014 mmol) was dissolved in a solution of ammonia in MeOH (7 M, 2 mL). The resulting mixture was stirred at room temperature for 2 hours before all volatiles were removed by rotavap. The residue was chromatographed (silica, dichloromethane-MeOH) to give the title compound (6 mg, 100%). ESIMS m/z=429.93 [M+H]⁺.

### Example 3

### Compound of Formula (I), Wherein Base is cytosine-1-yl, X is O, L² is CF₂, L¹ is CH₂, L³ is Absent, R¹═R³═R⁴═H, R²=Me, R^{3a}═OH, W¹═W²═OH

Into a solution of the compound of Example 2 (4 mg, 0.01 mmol) in acetonitrile (2 mL) and DMF (0.1 mL) was added trimethylsilyl bromide (132 mg). The resulting mixture was stirred at room temperature for 2 hours before all volatiles were removed. The residue was purified by HPLC (C-18 column, acetonitrile-20 mM ammonium bicarbonate in water) to give the title compound (2.7 mg, 72%). ESIMS m/z=396.39 [M+Na]⁺.

### Example 4

### Compound of Formula (I), Wherein Base is uracil-1-yl, X is O, L² is CF₂, L¹ is CH₂, L³ is Absent, R¹═R³═R⁴═H, R²=Me, R^{3a}═OAc, W¹═W²═OEt

A solution of the compound of Example 1 (14 mg, 0.024 mmol) in AcOH (0.8 mL) and water (0.2 mL) was stirred at 80° C. for 12 hours before all volatiles were removed by rotavap. The residue was chromatographed (silica, CH₂Cl₂-MeOH) to give the title compound (8 mg, 70%). ESIMS m/z=494.80 [M+Na]⁺.

### Example 5

### Compound of Formula (I), Wherein Base is uracil-1-yl, X is O, L² is CF₂, L¹ is CH₂, L³ is Absent, R¹═R³═R⁴═H, R²=Me, R^{3a}═OH, W¹═W²═OEt

The compound of Example 4 (8 mg, 0.017 mmol) was dissolved in a solution of ammonia in MeOH (7 molar, 2 mL). The resulting mixture was stirred at room temperature for 2 hours before all volatiles were removed by rotavap. The residue was chromatographed (silica, dichloromethane-MeOH) to give the title compound (8 mg, ca. 100%). ESIMS m/z=431.12 [M+H]⁺.

### Example 6

### Compound of Formula (I), Wherein Base is uracil-1-yl, X is O, L² is CF₂, L¹ is CH₂, L³ is Absent, R¹═R³═R⁴═H, R²=Me, R^{3a}═OH, W¹═W²═OH

Into a solution of the compound of Example 5 (5 mg, 0.012 mmol) in acetonitrile (1 mL) and DMF (0.1 mL) was added trimethylsilyl bromide (0.05 mL). The resulting mixture was stirred at room temperature for 2 hours before all volatiles were removed by rotavap. The residue was purified by HPLC (C-18 column, acetonitrile-20 mmol ammonium bicarbonate in water) to give the title compound (1.8 mg, 40%). ESIMS m/z=375.08 [M+H]⁺.

### Example 7

### Compound of Formula (I), Wherein Base is uracil-1-yl, X is O, L² is CF₂, L¹ is CH₂, L³ is Absent, R¹═R³═R⁴═H, R²=Me, R^{3a}═OC(O)CH₂CH₂C(O)Me, W¹═W²═OH

Into a solution of the compound of example 5 (38 mg, 0.09 mmol) in pyridine (2 mL) was added DMAP (5 mg) and levulinic anhydride (0.7 mL, 0.36 mmol, prepared according to Journal of Organic Chemistry, 2004, 69, 6310). The resulting mixture was stirred at room temperature for 1 hour before being partitioned between aqueous NaHCO₃ and EtOAc. The organic phase was washed with aqueous NaHCO₃, dried (Na₂SO₄) and evaporated. The residue was chromatographed (silica, hexane-EtOAc) to give a light yellow oil, which was redissolved in acetonitrile (3 mL) followed by addition of trimethylsilyl bromide (0.53 mL). The resulting mixture was stirred at room temperature for 2 hours before all volatiles were removed by rotavap. The residue was purified by HPLC (C-18 column, acetonitrile-20 mmol ammonium bicarbonate in water) to give the title compound (32 mg, 76%). ESIMS m/z=473.05 [M+H]⁺.

### Example 8

### Compound of Formula (I), Wherein Base is uracil-1-yl, X is O, L² is CF₂, L¹ is CH₂, L³ is Absent, R¹═R³═R⁴═H, R²=Me, R^{3a}═OC(O)CH₂CH₂C(O)Me, W¹═OPh, W²═OH

Into a solution of the compound of Example 7 (32 mg, 0.068 mmol) in CH₂Cl₂ (3 mL) was added oxalyl chloride (86 mg, 0.68 mmol) and DMF (0.002 mL). The resulting mixture was stirred at room temperature for 1 hour before all volatiles were removed by rotavap. The residue was redissolved in CH₂Cl₂ (3 mL) and was added phenol (64 mg) and Et₃N (0.2 mL). After 2 hours, the mixture was partitioned between aqueous NaHCO₃ and EtOAc. The organic phase was washed with water, dried (Na₂SO₄) and evaporated. The residue was chromatographed (silica, hexane-EtOAc) to give the desired compound, which was redissolved in pyridine (3 mL), Et₃N (0.3 mL) and water (0.3 mL). The resulting mixture was stirred at room temperature for 3 hours before all volatiles were removed by rotavap. The residue was purified by HPLC (C-18 column, acetonitrile-20 mM ammonium bicarbonate in water) to give the title compound (14 mg, 36%). ESIMS m/z=549.06 [M+H]⁺.

### Example 9

### Compound of Formula (I), Wherein Base is uracil-1-yl, X is O, L² is CF₂, L¹ is CH₂, L³ is Absent, R¹═R³═R⁴═H, R²=Me, R^{3a}═OC(O)CH₂CH₂C(O)Me, W¹═OPh, W²═(S)—NH—CH(Me)CO₂Me

Into a solution of the compound of Example 8 (14 mg, 0.026 mmol) in CH₂Cl₂ (3 mL) was added oxalyl chloride (0.1 mL) and DMF (0.003 mL). The resulting mixture was stirred at room temperature for 1 hour before all volatiles were removed by rotavap. The residue was redissolved in pyridine (3 mL) and was added L-alanine methyl ester (50 mg). The resulting mixture was stirred at room temperature for 1 hour before all volatiles were removed. The residue was chromatographed (silica, CH₂Cl₂-MeOH) to give the title compound (5 mg, 30%). ESIMS m/z=634.34 [M+H]⁺.

### Example 10

### Compound of Formula (I), Wherein Base is uracil-1-yl, X is O, L² is CF₂, L¹ is CH₂, L³ is Absent, R¹═R³═R⁴═H, R²=Me, W¹═OPh, W²═(S)—NH—CH(Me)CO₂Me

Into a solution of the compound of Example 9 (5 mg, 0.0078 mmol) in pyridine (1 mL) was added a mixture (1 mL, 24% hydrazine in water/pyridine/AcOH=2:4:3). The resulting mixture was stirred at room temperature for 5 minutes before being partitioned between aqueous NaHCO₃ and EtOAc. The organic phase was dried (Na₂SO₄) and evaporated. The residue was chromatographed (silica, dichloromethane-MeOH) to give the title compound (1.5 mg, 36%). ESIMS m/z=558.26 [M+Na]⁺.

### Example 11

### Compound of Formula (I), Wherein Base is N⁴-levulinoylcytosin-1-yl, X is O, L² is CF₂, L¹ is CH₂, L³ is Absent, R¹═R³═R⁴═H, R²=Me, R═OC(O)CH₂CH₂C(O)Me, W¹═W²═OH

Into a solution of the compound of Example 2 (44 mg, 0.10 mmol) in pyridine (5 mL) was added DMAP (7 mg) and levulinic anhydride (0.7 mL, 0.36 mmol, prepared according to Journal of Organic Chemistry, 2004, 69, 6310). The resulting mixture was stirred at room temperature for 1 hour before being partitioned between aqueous NaHCO₃ and EtOAc. The organic phase was washed with aqueous NaHCO₃, dried (Na₂SO₄) and evaporated. The residue was chromatographed (silica, hexane-EtOAc) to give the desired compound, which was redissolved in acetonitrile (5 mL) followed by addition of trimethylsilyl bromide (0.58 mL). The resulting mixture was stirred at room temperature for 2 hours before all volatiles were removed by rotavap. The residue was purified by HPLC (C-18 column, acetonitrile-20 mmol ammonium bicarbonate in water) to give the title compound (38 mg, 66%). ESIMS m/z=570.24 [M+H]⁺.

### Example 12

### Compound of Formula (I), Wherein Base is N⁴-levulinoylcytosin-1-yl, X is O, L² is CF₂, L¹ is CH₂, L³ is Absent, R¹═R³═R⁴═H, R²=Me, R^{3a}═OC(O)CH₂CH₂C(O)Me, W¹═OPh, W²═OH

Into a solution of the compound of Example 11 (36 mg, 0.063 mmol) in CH₂Cl₂ (3 mL) was added oxalyl chloride (0.11 mL) and DMF (0.003 mL). The resulting mixture was stirred at room temperature for 45 minutes before all volatiles were removed by rotavap. The residue was redissolved in CH₂Cl₂ (3 mL) and was added phenol (60 mg) and Et₃N (0.18 mL). After 2 hours, the mixture was partitioned between aqueous NaHCO₃ and EtOAc. The organic phase was washed with aqueous NaHCO₃, dried (Na₂SO₄) and evaporated. The residue was chromatographed (silica, hexane-EtOAc) to give the desired compound, which was redissolved in pyridine (3 mL), Et₃N (0.3 mL) and water (0.3 mL). The resulting mixture was stirred at room temperature for 3 hours before all volatiles were removed by rotavap. The residue was purified by HPLC (C-18 column, acetonitrile-20 mmol ammonium bicarbonate in water) to give the title compound (16 mg, 35%). ESIMS m/z=646.33 [M+H]⁺.

### Example 13

### Compound of Formula (I), Wherein Base is N⁴-levulinoylcytosin-1-yl, X is O, L² is CF₂, L¹ is CH₂, L³ is Absent, R¹═R³═R⁴═H, R²=Me, R^{3a}═OC(O)CH₂CH₂C(O)Me, W¹═OPh, W²═(S)—NH—CH(Me)CO₂Me

Into a solution of the compound of example 12 (15 mg, 0.024 mmol) in CH₂Cl₂ (2 mL) was added oxalyl chloride (0.04 mL) and DMF (0.002 mL). The resulting mixture was stirred at room temperature for 1 hour before all volatiles were removed by rotavap. The residue was redissolved in pyridine (2 mL) and was added L-alanine methyl ester (50 mg). The resulting mixture was stirred at room temperature for 1 hour before all volatiles were removed. The residue was chromatographed (silica, dichloromethane-MeOH) to give the title compound (3 mg, 18%). ESIMS m/z=731.41 [M+H]⁺.

### Example 14

### Compound of Formula (I), Wherein Base is cytosin-1-yl, X is O, L² is CF₂, L¹ is CH₂, L³ is Absent, R¹═R³═R⁴═H, R²=Me, R^{3a}═OH, W¹═OPh, W²═(S)—NH—CH(Me)CO₂Me

Into a solution of the compound of Example 13 (3 mg) in pyridine (1 mL) was added a mixture (1 mL, 24% hydrazine in water/pyridine/AcOH=2:4:3). The resulting mixture was stirred at room temperature for 5 minutes before being partitioned between aqueous NaHCO₃ and EtOAc. The organic phase was dried (Na₂SO₄) and evaporated. The residue was chromatographed (silica, dichloromethane-MeOH) to give the title compound (1.5 mg, 68%). ESIMS m/z=535.22 [M+H]⁺.

### Example 15

### Compound of Formula (I), Wherein Base is N⁴-benzoylcytosin-1-yl, X is O, L² is CHF, L¹ is CH₂, L³ is Absent, R¹═R³═R⁴═H, R²=Me, R^{3a}═OBz, W¹═W²═OEt

Step 15a. A mixture of N⁴-benzoylcytidine (40.0 g, 0.115 mol), pyridine (350 mL) and 1,3-dichloro-1,1,3,3-tetraisopropyldisiloxane (36.84 mL, 0.115 mol) was stirred at room temperature overnight. The volatiles were evaporated and the residue was partitioned (EtOAc-water). The organics were washed with HCl solution (0.1 M), water, brine, dried (Na₂SO₄), filtered and evaporated. The residue was chromatographed (silica, hexanes-ethyl acetate) to give the desired compound as a white foam (56.60 g, 83%). ESIMS m/z=590.18 [M+H]⁺.

Step 15b. A mixture of the compound from step 15a (56.6 g, 96.0 mmol) and DMSO (44.3 mL, 0.624 mol) in THF (300 mL) was treated with trifluoroacetic anhydride (40.0 ml, 0.288 mol) at −20° C. for 2 hours. Triethylamine (60.2 ml, 0.432 mol) was then added slowly while keeping the internal temperature below −15° C. After addition, the cooling bath was removed and the reaction mixture was allowed to warm up to room temperature before being diluted with ethyl acetate. The organic layer was washed with H₂O (three times), brine, dried (Na₂SO₄), filtered and evaporated. The residue was chromatographed (silica, hexanes-ethyl acetate) to give the desired compound as a yellow foam (47.6 g, 84%). ESIMS m/z=606.21 [M+H+H₂O]⁺.

Step 15c. A solution of the compound from step 15b (56.6 g, 96.0 mmol) in anhydrous ether (700 mL) at −78° C. was added MeLi (1.6 M in Et₂O, 253.0 mL, 0.405 mol) dropwise. The resultant mixture was stirred at −78° C. for 3 hours. It was quenched by 1 M NH₄Cl (500 mL). Then the cooling bath was removed and the reaction mixture was allowed to warm up to room temperature before being diluted with ethyl acetate. The organic layer was washed with H₂O, brine, dried (Na₂SO₄), filtered and concentrated to give the crude desired compound as a brown foam (47.1 g), which was used directly for next step. ESIMS m/z=604.17 [M+H]⁺.

Step 15d. A mixture of the compound from step 15c (47.1 g, 78.0 mmol) in THF (750 mL) was added triethylamine (87.0 mL, 0.624 mol) and triethylamine trihydrofluride (50.9 ml, 0.312 mol) was stirred at room temperature overnight before being concentrated. The residue was chromatographed (silica, dichloromethane/methanol) to give the desired compound as a black foam (29.1 g, contains some salt). ESIMS m/z=362.05 [M+H]⁺.

Step 15e. The compound from step 15d (2.000 g, 5.535 mmol) was co-evaporated with pyridine once before being taken up in dry pyridine (25 mL), into which DMT-Cl (2.248 g, 6.642 mmol) was added. The mixture was stirred at room temperature for 3 hours before being quenched by MeOH (3 ml) at 0° C. The volatiles were evaporated. The residue was chromatographed (silica, dichloromethane/methanol) to give the desired compound as a yellow solid (1.160 g, 32%). ESIMS m/z=664.02 [M+H]⁺.

Step 15f. A mixture of the compound from step 15e (1.160 g, 1.748 mmol) and imidazole (0.357 g, 5.243 mmol) in DMF (12 mL) was treated with TBSCl (0.395 g, 2.622 mmol) and DMAP (0.214 g, 1.748 mmol) at room temperature overnight before more imidazole (0.297 g), TBSCl (0.658 g) and DMAP (107 mg) were added in two portions in 8 hours. The solution was stirred at room temperature overnight being quenched by MeOH (1.5 mL) and partitioned (EtOAc-water). The organics were washed with brine twice, dried (Na₂SO₄), filtered and evaporated. The residue was chromatographed (silica, hexanes-ethyl acetate, with 0.5% EtOH in ethyl acetate) to give the desired compound as a yellow foam (0.775 g, 81%). ESIMS m/z=778.19 [M+H]⁺.

Step 15g. A solution of the compound from step 15f (0.770 g, 0.990 mmol) in CH₂Cl₂ (10 mL) was treated with TsOH hydrate (0.198 g, 1.039 mmol) at room temperature for 2.5 hours. It was quenched by excess amount of triethylamine and concentrated. The residue was purified by flash column chromatography (silica, CH₂Cl₂/methanol) to give the desired compound as a yellow solid (0.417 g, 89%). ESIMS m/z=476.08 [M+H]⁺.

Step 15h. A solution of the compound from step 15g (0.340 g, 0.715 mmol) in CH₂Cl₂ (8 mL) was treated with NaHCO₃ (0.546 g) and Dess-Martin periodinane (0.364 g, 0.856 mmol) at room temperature for 4 hours before being quenched by saturated Na₂S₂O₃ solution and partitioned (EtOAc-water). The organics were washed with brine, dried (Na₂SO₄), filtered and evaporated. The residue was chromatographed (silica, CH₂Cl₂/methanol) to give the desired compound as a slightly yellow solid (0.272 g, 80%). ESIMS m/z=474.06 [M+H]⁺.

Step 15i. A solution of (EtO)₂P(O)CHFP(O)(OEt)₂ (0.310 g, 1.013 mmol) in hexanes (5 mL) and toluene (3 mL) was treated with n-BuLi (2.5 M in hexanes, 0.41 mL, 1.013 mmol) at −78° C. for 20 min before a solution of the compound from step 15h (0.240 g, 0.507 mmol) in toluene (5 mL) was charged. Then the cooling was removed and the mixture was stirred at room temperature overnight before being quenched with saturated NH₄Cl solution and diluted (EtOAc). The organics were washed with saturated NH₄Cl, brine, dried (Na₂SO₄), filtered and evaporated. The residue was chromatographed (silica, CH₂Cl₂/methanol) to give the desired compounds as a white foam (0.275 g, 87%) and olefinic E/Z mixture. ESIMS m/z=626.18 [M+H]⁺.

Step 15j. A solution of the compound from step 15i (1.204 g, 1.924 mmol) in CH₃CN-THF (1/1, 30 mL) was treated with Et₃N (5.36 mL, 38.48 mol) and triethylamine trihydrofluride (3.14 ml, 19.24 mol) at 40° C. overnight before being concentrated. The residue was purified by flash column chromatography (silica, CH₂Cl₂/methanol) to give desired compound as a white solid (0.696 g, 71%). ESIMS m/z=512.08 [M+H]⁺.

Step 15k. The compound from step 15j (0.680 g, 1.330 mmol) was treated with 2 N ammonia in methanol (20 mL) at room temperature for 3 hours before charging 7 N ammonia in methanol (18 mL). The reaction was followed by TLC until completion. It was concentrated to give crude desired compound as a white solid (0.609 g), which was used directly for next step. ESIMS m/z=408.13 [M+H]⁺.

Step 15l. A solution of the compound from step 15 k (0.609 g, maximum 1.330 mmol) in EtOH (200 proof, 140 mL) was hydrogenated with a H₂ ballon in the presence of 10% Pd/C (0.120 g) at room temperature for 1.5 hours before filtration through celite. The filtrate was concentrated to give desired compound as a white solid (0.580 g), which was used directly for next step. ESIMS m/z=410.14 [M+H]⁺.

Step 15m. A solution of the compound from step 151 (0.580 g) in pyridine (30 mL) was treated with benzoyl chloride (0.36 mL, 3.117 mol) at room temperature for 2 hours. More benzoyl chloride (0.18 mL) was added and the mixture was stirred for another hour. It was quenched with MeOH (2 mL) and concentrated. The residue was chromatographed (silica, hexanes-ethyl acetate, with 0.5% EtOH in ethyl acetate) to give the desired compounds as a semi-solid and epimeric mixture (0.560 g, 67% over 3 steps). ESIMS m/z=618.15 [M+H]⁺.

Step 15n. A solution of the compound from step 15m (0.454 g, 0.735 mmol) in CH₂Cl₂ (7 mL) at 0° C. was treated with Py.(HF)ₙ (7 mL) and DAST (0.19 mL, 1.47 mmol). Then the cooling bath was removed and solution was stirred at room temperature for 2 hours and more DAST (0.09 mL) was added. After 1 hr it was quenched carefully by saturated NaHCO₃ at 0° C. The mixture was diluted with EtOAc. The organic layer was washed with saturated NaHCO₃ solution, brine, dried (Na₂SO₄), filtered and evaporated. The residue was chromatographed (silica, hexanes-ethyl acetate-EtOH) to give the title compounds as a white solid and epimeric mixture (33.0 mg, 7.3%). ESIMS m/z=620.29 [M+H]⁺.

### Example 16

### Compound of Formula (I), Wherein Base is cytosin-1-yl, X is O, L² is CHF, L¹ is CH₂, L³ is Absent, R¹═R³═R⁴═H, R²=Me, R^{3a}═OH, W¹═W²═OEt

The compound of Example 15 (13.1 mg, 0.0211 mmol) was treated with 7 N ammonia in methanol (4 mL) at room temperature for 3 hours. The mixture was concentrated. The residue was purified by flash column chromatography (silica, CH₂Cl₂/methanol) to give the title compound as a white solid and epimeric mixture (7.2 mg, 83%). ESIMS m/z=412.16 [M+H]⁺.

Alternatively, the compound of Example 21 (0.291 mg, 0.522 mmol) was dissolved in 7 N ammonia in methanol (10 mL). The solution was stirred at rt. More 7 N ammonia in methanol (3 mL) was added every hour. After totally 4 hr at rt, the solution was concentrated. The residue was purified by flash column chromatography (silica, dichloromethane/methanol) to give the same title compound as a white solid (216.5 mg, 100%).

### Example 17

### Compound of Formula (I), Wherein Base is cytosin-1-yl, X is O, L² is CHF, L¹ is CH₂, L³ is Absent, R¹═R³═R⁴═H, R²=Me, R^{3a}═OH, W¹═W²═OH

A solution of the compound of Example 16 (5.2 mg, 0.0126 mmol) in CH₃CN (1.0 mL) and DMF (0.2 mL) at 0° C. was treated with TMSBr (75 μL, 0.58 mmol) under N₂ at room temperature over weekend. It was co-evaporated with MeOH twice. The residue was purified by HPLC (C-18 column, 20 mM NH₄HCO₃ buffer/CH₃CN) to give the title compound as a white solid (4.7 mg, 96%). ESIMS m/z=356.07 [M+H]⁺.

### Example 18

### Compound of Formula (I), Wherein Base is uracil-1-yl, X is O, L² is CHF, L¹ is CH₂, L³ is Absent, R¹═R³═R⁴═H, R²=Me, R^{3a}═OBz, W¹═W²═OEt

A mixture of compound of Example 15 (17.0 mg, 0.0274 mmol) in 80% HOAc —H₂O (2 mL) was stirred at 80° C. overnight before being concentrated. The residue was co-evaporated with MeOH/H₂O (1/1, twice) and toluene (twice) to give the crude title compound. ESIMS m/z=517.16 [M+H]⁺.

### Example 19

### Compound of Formula (I), Wherein Base is uracil-1-yl, X is O, L² is CHF, L¹ is CH₂, L³ is Absent, R¹═R³═R⁴═H, R²=Me, R^{3a}═OH, W¹═W²═OEt

The crude compound of Example 18 (0.0274 mmol at most) was treated with 7 N ammonia in methanol (4 mL) at room temperature for 3.5 hours. It was concentrated. The residue was chromatographed (silica, CH₂Cl₂/methanol) to give the title compound as a white solid (8.6 mg, 76% over 2 steps). ESIMS m/z=413.07 [M+H]⁺. Alternatively, the crude compound of Example 22 was treated with ammonia in methanol to give the title compound. ESIMS m/z=413.13 [M+H]⁺.

### Example 20

### Compound of Formula (I), Wherein Base is uracil-1-yl, X is O, L² is CHF, L¹ is CH₂, L³ is Absent, R¹═R³═R⁴═H, R²=Me, R^{3a}═OH, W¹═W²═OH

A solution of the compound from Example 19 (5.8 mg, 0.0141 mmol) in CH₃CN (1.0 mL) at 0° C. was added TMS-Br (92.8 μL, 0.703 mmol) under N₂. After addition, the cooling bath was removed and the solution was stirred at room temperature overnight. It was co-evaporated with MeOH twice. The residue was purified by HPLC (C-18 column, 20 mM NH₄HCO₃ buffer/CH₃CN) to give desired compound as a white solid (3.5 mg, 76%). ESIMS m/z=357.04 [M+H]⁺.

### Example 21

### Compound of Formula (I), Wherein Base is N⁴-benzoylcytosin-1-yl, X is O, L² is CHF, L¹ is CH₂, L³ is Absent, R¹═R³═R⁴═H, R²=Me, R^{3a}═OAc, W¹═W²═OEt

Step 21a. To a solution of the compound from step 1d (1.100 g, 3.756 mmol) in CH₂Cl₂ (35 mL) at room temperature was added NaHCO₃ (1.800 g), followed by the addition of Dess-Martin periodinane (1.901 g, 4.483 mmol). The suspension was stirred at room temperature for 2 hours. The reaction was quenched by saturated Na₂S₂O₃ solution. The mixture was partitioned (EtOAc-water). The organics were washed with brine, dried (Na₂SO₄), filtered and evaporated. The residue was chromatographed (silica, hexanes-ethyl acetate) to give the desired compound as a colorless oil (1.073 g, 97%). ¹H NMR (CDCl₃, 500 MHz) 9.62 (d, J=1.5 Hz, 1H), 4.74 (d, J=6.3 Hz, 1H), 4.35 (m, 1H), 3.83 (dd, J=11.2, 6.8 Hz, 1H), 3.45 (s, 3H), 1.32 (d, J=22.0 Hz, 1H), 0.83 (s, 9H), 0.03 (s, 3H), 0.00 (s, 3H).

Step 21b. To a solution of (EtO)₂P(O)CHFP(O)(OEt)₂ (2.248 g, 7.342 mmol) in hexanes (36 mL) and toluene (24 mL) at −78° C. was added BuLi (2.5 M in hexanes, 2.94 mL, 7.342 mmol) dropwise. The mixture was stirred at −78° C. for 20 min before a solution of compound from step 21a (1.073 g, 3.671 mmol) in toluene (36 mL) was added dropwise. After the addition, the cooling bath was removed and the reaction mixture was stirred at rt overnight. The reaction was quenched with saturated NH₄Cl solution. The mixture was diluted with EtOAc. The organic layer was washed with saturated NH₄Cl solution, brine, dried (Na₂SO₄), filtered and evaporated. The residue was purified by flash column chromatography (silica, hexanes-ethyl acetate) to give the desired compound as a colorless oil and E/Z mixture (1.367 g, 84%). ESIMS m/z=445.19 [M+H]⁺.

Step 21c. To a solution of the compound from step 21b (1.367 g, 3.075 mmol) in EtOH (100 mL) was added 10% Pd/C (0.127 g). The suspension was evacuated and refilled with H₂ 3 times before being stirred at room temperature with a H₂ ballon for 24 hours. It was filtered through celite. The filtrate was concentrated to give the desired compound as a colorless oil (1.300 g, 95%), which was used directly for next step. ESIMS m/z=447.34 [M+H]⁺.

Step 21d. To a solution of the compound from step 21c (78.3 mg, 0.175 mmol) in dry CH₂Cl₂ (1 mL) at room temperature was added dropwise a solution of concentrated H₂SO₄ (0.017 mL) in Ac₂O (0.17 mL). The mixture was stirred at room temperature for 3 hours before being cooled with an ice-water bath. Pyridine (0.4 mL) and catalytic amount of DMAP was added at 0° C. The solution was stirred at room temperature for 1 hour before being diluted with EtOAc and water. The organic layer was washed saturated NaHCO₃ solution, brine, dried (Na₂SO₄), filtered and evaporated. The residue was chromatographed (silica, hexanes-ethyl acetate) to give the desired compound as a colorless oil (37.5 mg, 53%). ESIMS m/z=403.17 [M+H]⁺.

Step 21e. A suspension of N⁴-benzoylcytosine (0.430 g, 2.00 mmol) and (NH₄)₂SO₄ (10 mg) in 1,1,1,3,3,3-hexamethylsilazane (16 mL) was relaxed for 4 hours before it was allowed to cool down to room temperature. The solution was concentrated. The residue was co-evaporated with toluene twice and used directly for next step.

Step 21f. A mixture of the compound from step 21e (2.00 mmol at most) and the compound from step 21d (0.420 g, 1.044 mmol) in chlorobenzene (30 mL) in chlorobenzene (20 mL) was added Tin(IV) chloride (0.49 mL, 4.176 mmol) at 0° C. The resultant clear solution was stirred at 65° C. for 20 hours. It was cooled down and poured into a mixture of EtOAc and saturated NaHCO₃ solution. The aqueous layer was back-extracted with dichloromethane, EtOAc. The combined organic layers were washed with brine, dried (Na₂SO₄), filtered and evaporated. The residue was chromatographed (silica, hexanes-ethyl acetate) to give the title compound as a white solid (0.232 g, 40%). ESIMS m/z=558.16 [M+H]⁺.

### Example 22

### Compound of Formula (I), Wherein Base is uracil-1-yl, X is O, L² is CHF, L¹ is CH₂, L³ is Absent, R¹═R³═R⁴═H, R²=Me, R^{3a}═OBz, W¹═W²═OEt

A mixture of compound of Example 21 (0.101 g, 0.171 mmol) in 80% HOAc-H₂O (10 mL) was stirred at 80° C. overnight before being concentrated. The residue was co-evaporated with MeOH/H₂O (1/1, twice), toluene (twice), and used directly for next step. ESIMS m/z=455.13 [M+H]⁺.

### Example 23

### Compound of Formula (I), Wherein Base is uracil-1-yl, X is O, L² is CHF, L¹ is CH₂, L³ is Absent, R¹═R³═R⁴═H, R²=Me, R^{3a}═OC(O)CH₂CH₂C(O)Me, W¹═W²═OEt

To a solution of compound 4-8 (55.0 mg, 0.133 mmol) in pyridine (2 mL) was added DMAP (1.6 mg, 0.0133 mmol) and levulinic anhydride (1.25 mL, 0.667 mmol, freshly prepared according to Journal of Organic Chemistry, 2004, 69, 6310). The resultant mixture was stirred at room temperature for 2.5 hour before being quenched with i-PrOH (0.2 mL). The mixture was evaporated. The residue was chromatographed (silica, dichloromethane/methanol) to give the title compound as a colorless oil (67.0 mg). ESIMS m/z=511.19 [M+H]⁺.

### Example 24

### Compound of Formula (I), Wherein Base is uracil-1-yl, X is O, L² is CHF, L¹ is CH₂, L³ is Absent, R¹═R³═R⁴═H, R²=Me, R^{3a}═OC(O)CH₂CH₂C(O)Me, W¹═W²═OH

To a solution of the compound of Example 23 (41.0 mg, 0.0803 mmol) in CH₃CN (3.0 mL) at 0° C. was added TMS-Br (0.53 mL, 4.016 mmol) under N₂. The solution was stirred at rt overnight. The mixture was co-evaporated with MeOH twice. The residue was purified by HPLC (C-18 column, 20 mM NH₄HCO₃ buffer/CH₃CN) to give desired compound as a white solid (27.0 mg, 75% over 2 steps). ESIMS m/z=455.03 [M+H]⁺.

### Example 25

### Compound of Formula (I), Wherein Base is uracil-1-yl, X is O, L² is CHF, L¹ is CH₂, L³ is Absent, R¹═R³═R⁴═H, R²=Me, R^{3a}═OC(O)CH₂CH₂C(O)Me, W¹═OPh, W²═OH

To a solution of phenol (0.960 g, 10.2 mmol) in pyridine (10 mL) at −40° C. under N₂ was added thionyl chloride (0.350 mL, 4.80 mmol). The resultant white slurry was stirred at −40° C. for 1 hour before filtration. The filtrate was added into a flask containing compound of Example 24 (40.0 mg, 0.088 mmol). The mixture was stirred at rt for 4 hour and then at 65° C. for 0.5 hr before being concentrated. The residue was purified by HPLC (C-18 column, 20 mM NH₄HCO₃ buffer/CH₃CN) to give the title compound as a white foam (18.1 mg, 39%). ESIMS m/z=531.11 [M+H]⁺.

### Example 26

### Compound of Formula (I), Wherein Base is uracil-1-yl, X is O, L² is CHF, L¹ is CH₂, L³ is Absent, R¹═R³═R⁴═H, R²=Me, R^{3a}═OC(O)CH₂CH₂C(O)Me, W¹═OPh, W²═(S)—NH—CH(Me)CO₂Me

To a suspension of the compound of Example 25 (16.0 mg, 0.030 mmol) in CH₂Cl₂ (5 mL) at room temperature was added oxalyl chloride (0.1 mL) and DMF (0.0011 mL) under N₂. The resultant mixture was stirred at room temperature for 1 hour before all volatiles were removed by N₂. To the yellow residue was added a solution of L-alanine methyl ester (62.0 mg, 0.601 mmol) in pyridine (5 mL) at rt. The resultant mixture was stirred at room temperature for 2 hr before being concentrated. The residue was purified by flash column chromatography (silica, CH₂Cl₂/methanol) to give the desired compound as a yellow oil (5.1 mg). ESIMS m/z=616.19 [M+H]⁺.

### Example 27

### Compound of Formula (I), Wherein Base is uracil-1-yl, X is O, L² is CHF, L¹ is CH₂, L³ is Absent, R¹═R³═R⁴═H, R²=Me, R^{3a}═OH, W¹═OPh, W²═(S)—NH—CH(Me)CO₂Me

A mixture of H₂NNH₂.H₂O (98%, 0.25 mL), H₂O (0.75 mL), pyridine (2.0 mL) and HOAc (1.5 mL) was prepared and allowed to cool down to rt. To a solution of compound of Example 26 (5.1 mg) in pyridine (2 mL) was added the above mixture (2.0 mL). The resultant mixture was stirred at room temperature for 5 minutes before being concentrated. The residue was dissolved in EtOAc, washed with H₂O twice, brine, dried (Na₂SO₄) and evaporated. The residue was purified by preparative TLC (silica, dichloromethane/methanol) to give the desired compound as a light yellow solid (2.1 mg, 13% over 2 steps). ESIMS m/z=518.09 [M+H]⁺.

### Example 28

### Compound of Formula (I), Wherein Base is N4-cytosin-1-yl, X is O, L² is CHF, L¹ is CH₂, L³ is Absent, R¹═R³═R⁴═H, R²=Me, R^{3a}═OC(O)CH₂CH₂C(O)Me, W¹═W²═OEt

To a solution of the compound of Example 16 (216.5 mg, 0.526 mmol) in pyridine (8 mL) was added DMAP (6.4 mg, 0.0526 mmol) and levulinic anhydride (4.93 mL, 2.63 mmol, freshly prepared according to Journal of Organic Chemistry, 2004, 69, 6310). The resultant mixture was stirred at room temperature for 2 hr. More levulinic anhydride (4.00 mL) was added. After 1 hr at rt, the mixture was evaporated. The residue was chromatographed (silica, dichloromethane/methanol) to give the title product as a yellow oil (65.2 mg, 24%, ESIMS m/z=510.13 [M+H]⁺).

### Example 29

### Compound of Formula (I), Wherein Base is N4-cytosin-1-yl, X is O, L² is CHF, L¹ is CH₂, L³ is Absent, R¹═R³═R⁴═H, R²=Me, R^{3a}═OC(O)CH₂CH₂C(O)Me, W¹═W²═OH

To a solution of the compound of Example 28 (65.2 mg, 0.128 mmol) in CH₃CN (5.0 mL) at 0° C. was added TMS-Br (0.85 mL, 6.405 mmol) under N2. The solution was stirred at rt overnight. The mixture was co-evaporated with MeOH twice. The residue was purified by HPLC (C-18 column, 20 mM NH₄HCO₃ buffer/CH₃CN) to give title compound as a white foam (41.4 mg, 71%). ESIMS m/z=454.09 [M+H]⁺.

### Example 30

### Compound of Formula (I), Wherein Base is N⁴-cytosin-1-yl, X is O, L² is CHF, L¹ is CH₂, L³ is Absent, R¹═R³═R⁴═H, R²=Me, R^{3a}═OC(O)CH₂CH₂C(O)Me, W¹═OPh, W²═OH

To a solution of phenol (1.900 g, 20.2 mmol) in pyridine (20 mL) at −40° C. under N₂ was added thionyl chloride (0.700 mL, 9.60 mmol). The resultant white slurry was stirred at −40° C. for 1 hr before being filtered through a filter pad. The filtrate (10 mL) was added into a flask containing the compound of Example 29 (40.0 mg, 0.088 mmol). The mixture was stirred at rt for 1.5 hr and then at 40° C. for 1.5 hr before being concentrated. The residue was purified by HPLC (C-18 column, 20 mM NH₄HCO₃ buffer/CH₃CN) to give the title compound as a slightly yellow foam (11.3 mg, 26%). ESIMS m/z=530.11 [M+H]⁺.

### Example 31

### Compound of Formula (I), Wherein Base is N⁴-cytosin-1-yl, X is O, L² is CHF, L¹ is CH₂, L³ is Absent, R¹═R³═R⁴═H, R²=Me, R^{3a}═OC(O)CH₂CH₂C(O)Me, W¹═OPh, W²═(S)—NH—CH(Me)CO₂Me

To a suspension of compound of Example 30 (11.0 mg, 0.021 mmol) in dichloromethane (6 mL) at rt was added oxalyl chloride (0.09 mL) and DMF (0.001 mL) under N2. The resultant mixture was stirred at room temperature for 1.5 hour before all volatiles were removed by N2. To the yellow residue was added a solution of L-alanine methyl ester (70.0 mg, 0.601 mmol) in pyridine (5 mL) at rt. The resultant mixture was stirred at room temperature for 2 hr before being concentrated. The residue was purified by preparative TLC (silica, dichloromethane/methanol) to give the desired compound as a white solid (2.7 mg, 21%). ESIMS m/z=615.20 [M+H]⁺.

### Example 32

### Compound of Formula (I), Wherein Base is N⁴-cytosin-1-yl, X is O, L² is CHF, L¹ is CH₂, L³ is Absent, R¹═R³═R⁴═H, R²=Me, R^{3a}═OH, W¹═OPh, W²═(S)—NH—CH(Me)CO₂Me

A mixture of H₂NNH₂H₂O (98%, 0.25 mL), H₂O (0.75 mL), pyridine (2.0 mL) and HOAc (1.5 mL) was prepared and allowed to cool down to rt. To a solution of compound 4-15 (3.2 mg) in pyridine (0.5 mL) was added the above mixture (0.5 mL). The resultant mixture was stirred at room temperature for 10 minutes before being concentrated. The residue was purified by preparative TLC (silica, CH₂Cl₂/methanol) to give the desired compound as a yellow semi-solid (2.5 mg, 93%). ESIMS m/z=517.10 [M+H]⁺.

### Example 33

### Compound of Formula (II), Wherein Base is N⁴-cytosin-1-yl, X is O, R¹═R³═R⁴═H, R²=Me, R^{3a}═OBz, W¹═W²═OMe

Step 33a. Into a solution of 2′-α-methyl-2′-β-hydroxy-N⁴-benzoyl-2′-deoxycytidine (4-Amino-1-(3,4-dihydroxy-5-hydroxymethyl-3-methyl-tetrahydro-furan-2-yl)-1H-pyrimidin-2-one, prepared according to J. Med. Chem. 2005, 48, 5504; 6.25 g, 17.3 mmol) in anhydrous DMF (100 mL) were added imidazole (2.95 g, 43.3 mmol), TBDPSCl (4.95 mL, 19.1 mmol) and DMAP (0.423 g, 3.46 mmol) sequentially. The mixture was stirred at ambient temperature for 1 day before being quenched with aq. NaHCO₃ and partitioned between EtOAc and water. The organics were washed with brine, dried over sodium sulfate and evaporated. The residue was chromatographed (silica, hexanes-EtOAc) to give the desired compound (8.97 g, 86%) as a light yellow foam. ESIMS m/z=600.05 [M+H]⁺.

Step 33b. Into a solution of the compound from step 33a (8.78 g, 14.7 mmol) in pyridine (80 mL) at 0° C. was added BzCl (2.04 mL, 17.6 mmol). The mixture was gradually warmed up to ambient temperature and kept stirring until the disappearance of the starting material. The reaction was quenched by the addition of MeOH and the volatiles were evaporated. The residue was chromatographed (silica, hexanes-EtOAc) to give the desired compound (8.99 g, 87%) as a white foam. ESIMS m/z=704.06 [M+H]⁺.

Step 33c. Into a solution of the compound from step 33b (8.98 g, 12.8 mmol) in anhydrous toluene (150 mL) at −20° C. was added DAST (2.67 mL, 19.1 mmol) dropwise. The mixture was gradually warmed up to ambient temperature and kept stirring for 1 hour. The reaction was then cooled down to 0° C. and quenched by the slow addition of aq. NaHCO₃. The mixture was partitioned between EtOAc and water and the organics were washed with brine, dried over sodium sulfate and evaporated. The residue was chromatographed (silica, hexanes-EtOAc) to give the desired compound (2.68 g, 30%) as a light yellow foam. ESIMS m/z=706.26 [M+H]⁺.

Step 33d. Into a solution of the compound from step 33c (2.68 g, 3.79 mmol) in anhydrous THF (40 mL) were added AcOH (0.33 mL, 5.69 mmol) and TBAF (1M in THF, 11.4 mL, 11.4 mmol) sequentially. The reaction was stirred at ambient temperature for 14 hours before being quenched with aq. NH₄Cl. The mixture was partitioned between EtOAc and water and the organics were washed with brine, dried over sodium sulfate and evaporated. The residue was chromatographed (silica, hexanes-EtOAc) to give the desired compound (1.33 g, 75%) as a light yellow solid. ESIMS m/z=467.95 [M+H]⁺.

Step 33e. Into a solution of the compound from step 33d (50.0 mg, 0.107 mmol) in anhydrous DMF (1.5 mL) were added Lithium azide (15.7 mg, 0.321 mmol), PPh₃ (30.9 mg, 0.118 mμmol) and carbon tetrabromide (39.0 mg, 0.118 mmol) sequentially. The reaction was stirred at ambient temperature for 14 hours before the addition of aq. NaHCO₃. The mixture was partitioned between EtOAc and water and the organics were washed with brine, dried over sodium sulfate and evaporated. The residue was chromatographed (silica, hexanes-EtOAc) to give the desired compound (30.3 mg, 58%) as a white solid. ESIMS m/z=492.97 [M+H]⁺.

Step 33f. Into a solution of the compound from step 33e (30.3 mg, 60.9 μmol) in anhydrous THF (5 mL) was added P(OMe)₃ (7.2 μL, 60.9 μmol) dropwise. The resulting mixture was heated to reflux for 1 day before being evaporated to dryness. The residue was chromatographed (silica, hexanes-EtOAc) to give the desired compound (25.3 mg, 72%) as a colorless oil. ESIMS m/z=574.98 [M+H]⁺.

### Example 34

### Compound of Formula (II), Wherein Base is N⁴-cytosin-1-yl, X is O, R¹═R³═R⁴═H, R²=Me, R^{3a}═OH, W¹═OH, W²═OMe

A solution of the compound from step 33f (8.0 mg, 13.9 μmol) in NH₄OH (28%, 3 mL) in a sealed tube was heated up to 55° C. for 14 hours. The resulting mixture was evaporated to dryness and the residue was chromatographed (silica, dichloromethane-methanol) to give the desired compound (3.8 mg, 78%) as a colorless oil. ESIMS m/z=353.01 [M+H]⁺.

Although the invention has been described with respect to various preferred embodiments, it is not intended to be limited thereto, but rather those skilled in the art will recognize that variations and modifications may be made therein which are within the spirit of the invention and the scope of the appended claims.

What is claimed is:

1. A compound represented by Formula (I˜II): 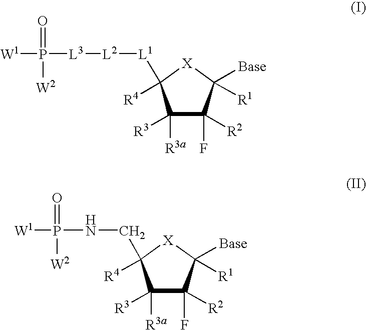
or a pharmaceutically acceptable salt, ester, stereoisomer, tautomer, solvate, prodrug, or combination thereof, wherein:
X is O, S, SO₂, or CH₂;
L¹ at each occurrence is —CR¹⁰R¹¹—, and L² at each occurrence is —CR¹²R¹³—, wherein one of R¹⁰, R¹¹, R¹², and R¹³ is a halogen or hydroxyl and the rest are selected from a group consisting of: hydrogen, deuterium, hydroxyl, or halogen; or R¹⁰ and R¹¹ or R¹² and R¹³ taken together with the carbon atom to which they are attached form a carbonyl or C₂-C₈ alkenylene group; or R¹⁰ and R¹² or R¹¹ and R¹³ taken together form a single bond; or R¹⁰ and R¹² or R¹¹ and R¹³ taken together with the carbon atom to which they are attached form a cyclopropane or oxirane ring;
L³ at each occurrence is each independently —CR¹⁰R¹¹— or absent;
R¹, R² and R⁴ at each occurrence are each independently selected from the group consisting of:
1) —C₁-C₈ alkyl, —C₂-C₈ alkenyl, —C₂-C₈ alkynyl or —C₃-C₈ cycloalkyl each containing 0, 1, 2, or 3 heteroatoms selected from O, S or N;
2) substituted —C₁-C₈ alkyl, substituted —C₂-C₈ alkenyl, substituted —C₂-C₈ alkynyl or substituted —C₃-C₈ cycloalkyl each containing 0, 1, 2, or 3 heteroatoms selected from O, S or N;
3) hydrogen;
4) deuterium;
5) —CN; and
6) halogen;
R³ and R^{3a} at each occurrence are each independently selected from the group consisting of:
1) hydrogen;
2) deuterium;
3) hydroxyl or protected hydroxyl;
4) halogen;
5) —CN;
6) —N₃;
7) —NR¹⁴R¹⁵, wherein R¹⁴ and R¹⁵ at each occurrence are each independently selected from the group consisting of: hydrogen and substituted or unsubstituted —C₁-C₈ alkyl;
8) —C₁-C₈ alkyl, —C₂-C₈ alkenyl, —C₂-C₈ alkynyl or —C₃-C₈ cycloalkyl each containing 0, 1, 2, or 3 heteroatoms selected from O, S or N; and
9) substituted —C₁-C₈ alkyl, substituted —C₂-C₈ alkenyl, substituted —C₂-C₈ alkynyl or substituted —C₃-C₈ cycloalkyl each containing 0, 1, 2, or 3 heteroatoms selected from O, S or N;
or R³ and R^{3a} taken together with the carbon atom to which they are attached form a group consisting of:
1) C═O;
2) C═NR¹⁴;
3) C═CR¹⁴R¹⁵;
4) C₃-C₈ cycloalkyl; and
5) 3-7 membered heterocyclic ring wherein containing at least one heteroatom from O, S or N;
W¹ and W² at each occurrence are each independently a group of the formula: 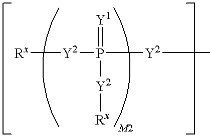
wherein:
each Y¹ is independently O, S, NR, ⁺N(O)(R), N(OR), ⁺N(O)(OR), or N—NR₂; wherein R is independently hydrogen, halogen, C₁-C₈ alkyl, substituted C₁-C₈ alkyl, C₂-C₈ alkenyl, substituted C₂-C₈ alkenyl, C₂-C₈ alkynyl, substituted C₂-C₈ alkynyl, aryl, substituted aryl, heterocyclic, substituted heterocyclic or a protecting group;
each Y² is independently a bond, O, CR₂, NR, ⁺N(O)(R), N(OR), ⁺N(O)(OR), N—NR₂, S, S—S, S(O), or S(O)₂;
M2 is 0, 1 or 2;
each R^{x} is independently R^{y}, a protecting group, or a group of the formula: 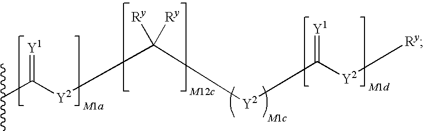
wherein:
M1a, M1c, and M1d are independently 0 or 1;
M12c is 0, 1, 2, 3, 4, 5, 6, 7, 8, 9, 10, 11 or 12;
or when taken together, two R^{x} are optionally substituted C₂-C₈ alkylene thereby forming a phosphorous-containing heterocycle;
each R^{y} is independently H, F, Cl, Br, I, OH, R, —C(═Y¹)R, —C(═Y¹)OR, —C(═Y¹)N(R)₂, —N(R)₂, —⁺N(R)₃, —SR, —S(O)R, —S(O)₂R, —S(O)(OR), —S(O)₂(OR), —OC(═Y¹)R, —OC(═Y¹)OR, —OC(═Y¹)(N(R)₂), —SC(═Y¹)R, —SC(═Y¹)OR, —SC(═Y¹)(N(R)₂), —N(R)C(═Y¹)R, —N(R)C(═Y¹)OR, or —N(R)C(═Y¹)N(R)₂, amino (—NH₂), ammonium (—NH₃⁺), alkylamino, dialkylamino, trialkylammonium, C₁-C₈ alkyl, C₁-C₈ alkyl halide, carboxylate, sulfamate, C₁-C₈ alkyl-hydroxyl, C₁-C₈ alkyl-thiol, sulfonamide (—SO₂NR₂), nitrile (—CN), azido (—N₃), nitro (—NO₂), C₁-C₈ alkoxy (—OR), a protecting group, or W³; or when taken together, two R^{y} on the same carbon atom forms a carbocyclic ring of 3-7 carbon atoms;
W³ is W⁴ or W⁵; wherein W⁴ is R, —C(Y¹)R^{y}, C(Y¹)W⁵, —SO₂R^{y}, or —SO₂W⁵; and W⁵ is a substituted or unsubstituted alicyclic, a substituted or unsubstituted aryl, or a substituted or unsubstituted heterocyclic group;
Base is a heterocycle containing at least one nitrogen, preferably pyrimidine or purine base of the general formula of (III)-(IV): 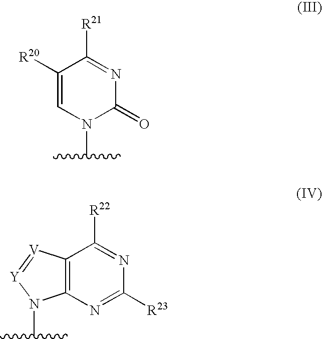
wherein:
W, Y and V are each independently N, CH, or CR¹⁶; wherein R¹⁶ is a halogen, C₁-C₈ alkyl, ary, acyl;
each R²⁰ is independently selected from the group consisting of:
1) —C₁-C₈ alkyl, —C₂-C₈ alkenyl, —C₂-C₈ alkynyl or —C₃-C₈ cycloalkyl each containing 0, 1, 2, or 3 heteroatoms selected from O, S or N;
2) substituted —C₁-C₈ alkyl, substituted —C₂-C₈ alkenyl, substituted —C₂-C₈ alkynyl or substituted —C₃-C₈ cycloalkyl each containing 0, 1, 2, or 3 heteroatoms selected from O, S or N;
3) hydrogen;
4) deuterium;
5) —CN;
6) halogen; and
7) —C(O)R¹⁷, wherein R¹⁷ is —C₁-C₈ alkyl, OH, OR¹⁴, or NR¹⁴R¹⁵; and R²¹, R²² and R²³ are independently a hydrogen, halogen (F, Cl, Br, I), OH, OR¹⁴, SH, SR¹⁴, NH₂, NHR¹⁴, NR¹⁴R¹⁵, OCOR¹⁴, OCOOR¹⁴, NHCOR¹⁴ or NHCOOR¹⁴.

2. A compound of claim 1 wherein L³ is absent, and R¹, R³ and R⁴ are hydrogen.

3. A compound of claim 1 wherein L³ is absent, R² is methyl, R³ is hydrogen, and R^{3a} is hydroxyl.

4. A compound of claim 1 wherein L³ is absent, R¹, R³ and R⁴ are hydrogen, R² is methyl, and R^{3a} is hydroxyl.

5. A compound of claim 1 wherein L³ is absent, R¹, R³ and R⁴ are hydrogen, R² is methyl, R^{3a} is hydroxyl, and Base is cytosine.

6. A compound of claim 1 wherein L³ is absent, R¹, R³ and R⁴ are hydrogen, L² is —CH₂—, L¹ is —CHF— or —CF₂—.

7. A compound of claim 1 wherein L³ is absent, R¹, R³ and R⁴ are hydrogen, L¹ is —CH₂—, L² is —CHF— or —CF₂—.

8. A compound of claim 1 wherein L³ is absent, R¹, R³ and R⁴ are hydrogen, R² is methyl, and R^{3a} is hydroxyl; and the structure 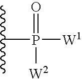
of Formula (I) or (II) is selected from the structures: 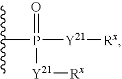
wherein Y²¹ is O or N(R); R and R^{x} are as previously defined; or 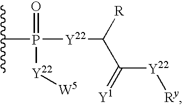
wherein Y²² is O, S or N(R); R, Y¹, W⁵ and R^{x} are as previously defined; or 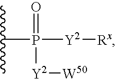
wherein W⁵⁰ is a substituted or unsubstituted aryl such as phenyl or substituted phenyl; Y² and R^{x} are as previously defined; or 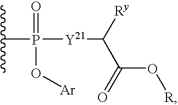
wherein Y²¹, R and R^{y} are as previously defined; or 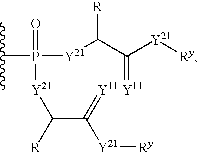
wherein Y¹¹ is O or S; each Y²¹ is independently as previously defined.

9. A pharmaceutical composition comprising a compound or a combination of compounds according to claim 1 or a pharmaceutically acceptable salt, stereoisomer, tautomer, prodrug, salt of a prodrug, or combination thereof, in combination with a pharmaceutically acceptable carrier or excipient.

10. The pharmaceutical composition of claim 9 further comprising a compound selected from the group consisting of cytokines, protease inhibitors, antiviral agents, proteases, caspase inhibitors, antibodies and protease inhibitors.

11. A method of inhibiting the replication of an RNA or DNA-containing virus comprising contacting said virus with a therapeutically effective amount of a compound or combination of compounds of claim 1, or a pharmaceutically acceptable salt, stereoisomer, tautomer, prodrug, salt of a prodrug, or combination thereof.

12. A method of preventing or treating abnormal cellular proliferation, a viral infection, or a symptom thereof in a subject in need thereof comprising administering to the subject a therapeuctially effective amount of a compound or combination of compounds of claim 1, or a pharmaceutically acceptable salt, stereoisomer, tautomer, prodrug, salt of a prodrug, or combination thereof.

13. The method of claim 12 wherein the virus is human immunodeficiency virus (HIV), hepatitis C virus (HCV) or hepatitis B virus (HBV).

14. A compound according to claim 1 selected from the group consisting of:
Compound of Formula (I), wherein Base is N⁴-benzoylcytosin-1-yl, X is O, L² is CF₂, L¹ is CH₂, L³ is absent, R¹═R³═R⁴═H, R²=Me, R^{3a}═OAc, W¹═W²═OEt;
Compound of Formula (I), wherein Base is cytosine-1-yl, X is O, L² is CF₂, L¹ is CH₂, L³ is absent, R¹═R³═R⁴═H, R²=Me, R^{3a}═OH, W¹═W²═OEt;
Compound of Formula (I), wherein Base is cytosine-1-yl, X is O, L² is CF₂, L¹ is CH₂, L³ is absent, R¹═R³═R⁴═H, R²=Me, R^{3a}═OH, W¹═W²═OH;
Compound of Formula (I), wherein Base is uracil-1-yl, X is O, L² is CF₂, L¹ is CH₂, L³ is absent, R¹═R³═R⁴═H, R²=Me, R^{3a}═OAc, W¹═W²═OEt;
Compound of Formula (I), wherein Base is uracil-1-yl, X is O, L² is CF₂, L¹ is CH₂, L³ is absent, R¹═R³═R⁴═H, R²=Me, R^{3a}═OH, W¹═W²═OEt;
Compound of Formula (I), wherein Base is uracil-1-yl, X is O, L² is CF₂, L¹ is CH₂, L³ is absent, R¹═R³═R⁴═H, R²=Me, R^{3a}═OH, W¹═W²═OH;
Compound of Formula (I), wherein Base is uracil-1-yl, X is O, L² is CF₂, L¹ is CH₂, L³ is absent, R¹═R³═R⁴═H, R²=Me, R^{3a}═OC(O)CH₂CH₂C(O)Me, W¹═W²═OH;
Compound of Formula (I), wherein Base is uracil-1-yl, X is O, L² is CF₂, L¹ is CH₂, L³ is absent, R¹═R³═R⁴═H, R²=Me, R^{3a}═OC(O)CH₂CH₂C(O)Me, W¹═OPh, W²═OH;
Compound of Formula (I), wherein Base is uracil-1-yl, X is O, L² is CF₂, L¹ is CH₂, L³ is absent, R¹═R³═R⁴═H, R²=Me, R^{3a}═OC(O)CH₂CH₂C(O)Me, W¹═OPh, W²═(S)—NH—CH(Me)CO₂Me;
Compound of Formula (I), wherein Base is uracil-1-yl, X is O, L² is CF₂, L¹ is CH₂, L³ is absent, R¹═R³═R⁴═H, R²=Me, R^{3a}═OH, W¹═OPh, W²═(S)—NH—CH(Me)CO₂Me;
Compound of Formula (I), wherein Base is N⁴-levulinoylcytosin-1-yl, X is O, L² is CF₂, L¹ is CH₂, L³ is absent, R¹═R³═R⁴═H, R²=Me, R^{3a}═OC(O)CH₂CH₂C(O)Me, W¹═W²═OH;
Compound of Formula (I), wherein Base is N⁴-levulinoylcytosin-1-yl, X is O, L² is CF₂, L¹ is CH₂, L³ is absent, R¹═R³═R⁴═H, R²=Me, R^{3a}═OC(O)CH₂CH₂C(O)Me, W¹═OPh, W²═OH;
Compound of Formula (I), wherein Base is N⁴-levulinoylcytosin-1-yl, X is O, L² is CF₂, L¹ is CH₂, L³ is absent, R¹═R³═R⁴═H, R²=Me, R^{3a}═OC(O)CH₂CH₂C(O)Me, W¹═OPh, W²═(S)—NH—CH(Me)CO₂Me;
Compound of Formula (I), wherein Base is cytosin-1-yl, X is O, L² is CF₂, L¹ is CH₂, L³ is absent, R¹═R³═R⁴═H, R²=Me, R^{3a}═OH, W¹═OPh, W²═(S)—NH—CH(Me)CO₂Me;
Compound of Formula (I), wherein Base is N⁴-benzoylcytosin-1-yl, X is O, L² is CHF, L¹ is CH₂, L³ is absent, R¹═R³═R⁴═H, R²=Me, R^{3a}═OBz, W¹═W²═OEt;
Compound of Formula (I), wherein Base is cytosin-1-yl, X is O, L² is CHF, L¹ is CH₂, L³ is absent, R¹═R³═R⁴═H, R²=Me, R^{3a}═OH, W¹═W²═OEt;
Compound of Formula (I), wherein Base is cytosin-1-yl, X is O, L² is CHF, L¹ is CH₂, L³ is absent, R¹═R³═R⁴═H, R²=Me, R^{3a}═OH, W¹═W²═OH;
Compound of Formula (I), wherein Base is uracil-1-yl, X is O, L² is CHF, L¹ is CH₂, L³ is absent, R¹═R³═R⁴═H, R²=Me R^{3a}═OBz, W¹═W²═OEt;
Compound of Formula (I), wherein Base is uracil-1-yl, X is O, L² is CHF, L¹ is CH₂, L³ is absent, R¹═R³═R⁴═H, R²=Me, R^{3a}═OH, W¹═W²═OEt;
Compound of Formula (I), wherein Base is uracil-1-yl, X is O, L² is CHF, L¹ is CH₂, L³ is absent, R¹═R³═R⁴═H, R²=Me, R^{3a}═OH, W¹═W²═OH;
Compound of Formula (I), wherein Base is N⁴-benzoylcytosin-1-yl, X is O, L² is CHF, L¹ is CH₂, L³ is absent, R¹═R³═R⁴═H, R²=Me, R^{3a}═OAc, W¹═W²═OEt;
Compound of Formula (I), wherein Base is uracil-1-yl, X is O, L² is CHF, L¹ is CH₂, L³ is absent, R¹═R³═R⁴═H, R²=Me, R^{3a}═OBz, W¹═W²═OEt;
Compound of Formula (I), wherein Base is uracil-1-yl, X is O, L² is CHF, L¹ is CH₂, L³ is absent, R¹═R³═R⁴═H, R²=Me, R^{3a}═OC(O)CH₂CH₂C(O)Me, W¹═W²═OEt;
Compound of Formula (I), wherein Base is uracil-1-yl, X is O, L² is CHF, L¹ is CH₂, L³ is absent, R¹═R³═R⁴═H, R²=Me, R^{3a}═OC(O)CH₂CH₂C(O)Me, W¹═W²═OH;
Compound of Formula (I), wherein Base is uracil-1-yl, X is O, L² is CHF, L¹ is CH₂, L³ is absent, R¹═R³═R⁴═H, R²=Me, R^{3a}═OC(O)CH₂CH₂C(O)Me, W¹═OPh, W²═OH;
Compound of Formula (I), wherein Base is uracil-1-yl, X is O, L² is CHF, L¹ is CH₂, L³ is absent, R¹═R³═R⁴═H, R²=Me, R^{3a}═OC(O)CH₂CH₂C(O)Me, W¹═OPh, W²═(S)—NH—CH(Me)CO₂Me;
Compound of Formula (I), wherein Base is uracil-1-yl, X is O, L² is CHF, L¹ is CH₂, L³ is absent, R¹═R³═R⁴═H, R²=Me, R^{3a}═OH, W¹═OPh, W²═(S)—NH—CH(Me)CO₂Me;
Compound of Formula (I), wherein Base is N⁴-cytosin-1-yl, X is O, L² is CHF, L¹ is CH₂, L³ is absent, R¹═R³═R⁴═H, R²=Me, R^{3a}═OC(O)CH₂CH₂C(O)Me, W¹═W²═OEt;
Compound of Formula (I), wherein Base is N⁴-cytosin-1-yl, X is O, L² is CHF, L¹ is CH₂, L³ is absent, R¹═R³═R⁴═H, R²=Me, R^{3a}═OC(O)CH₂CH₂C(O)Me, W¹═W²═OH;
Compound of Formula (I), wherein Base is N⁴-cytosin-1-yl, X is O, L² is CHF, L¹ is CH₂, L³ is absent, R¹═R³═R⁴═H, R²=Me, R^{3a}═OC(O)CH₂CH₂C(O)Me, W¹═OPh, W²═OH;
Compound of Formula (I), wherein Base is N4-cytosin-1-yl, X is O, L² is CHF, L¹ is CH₂, L³ is absent, R¹═R³═R⁴═H, R²=Me, R^{3a}═OC(O)CH₂CH₂C(O)Me, W¹═OPh, W²═(S)—NH—CH(Me)CO₂Me;
Compound of Formula (I), wherein Base is N4-cytosin-1-yl, X is O, L² is CHF, L¹ is CH₂, L³ is absent, R¹═R³═R⁴═H, R²=Me, R^{3a}═OH, W¹═OPh, W²═(S)—NH—CH(Me)CO₂Me;
Compound of Formula (II), wherein Base is N⁴-cytosin-1-yl, X is O, R¹═R³═R⁴═H, R²=Me, R^{3a}═OBz, W¹═W²═OMe;
Compound of Formula (II), wherein Base is N⁴-cytosin-1-yl, X is O, R¹═R³═R⁴═H, R²=Me, R^{3a}═OH, W¹═OH, W²═OMe.
